(19) United States
(12) Patent Application Publication
(10) Pub. No.: US 2025/0161667 A1
(43) Pub. Date: May 22, 2025

(54) WEARABLE DISPOSABLE ELECTROTHERAPY DEVICE

(71) Applicant: Research Foundation of the City University of New York, New York, NY (US)

(72) Inventors: Marom Bikson, Brooklyn, NY (US); Mohamad FallahRad, Westbury, NY (US); Alexander Couzis, Yonkers, NY (US)

(21) Appl. No.: 18/955,575

(22) Filed: Nov. 21, 2024

Related U.S. Application Data
(60) Provisional application No. 63/676,474, filed on Jul. 29, 2024.
(60) Provisional application No. 63/623,007, filed on Jan. 19, 2024.
(60) Provisional application No. 63/601,506, filed on Nov. 21, 2023.

(51) Int. Cl. A61N 1/04; A61N 1/20

(52) U.S. Cl. CPC A61N 1/0496; A61N 1/20

(57) ABSTRACT

Disclosed is a wearable device. The wearable device includes a substrate layer. The substrate layer is configured to support a battery array comprising a plurality of battery cells, wherein least a first battery cell is electrically coupled to a second battery cell using a conductive interconnect. The wearable device also includes an interface layer coupled to the substrate layer. The interface layer is electrically coupled the battery array via a translayer interconnect. The interface configured to adhere the substrate to a body and to provide electrical stimulation to the body.

### CROSS-REFERENCE TO RELATED APPLICATIONS

This application claims the benefit of U.S. Provisional Patent Application No. 63/601,506, filed Nov. 21, 2023, U.S. Provisional Patent Application No. 63/623,007, filed Jan. 19, 2024, and U.S. Provisional Patent Application No. 63/676,474, filed Jul. 29, 2024, and entitled “WEARABLE DISPOSABLE ELECTROTHERAPY DEVICE,” each of which is hereby incorporated herein by reference in its entirety.

### BACKGROUND

Applications of non-invasive electrical stimulation span treatment of pain and headache, depression, addiction, age related cognitive decline, wound healing, aesthetic uses, bioelectronic medicine, and drug delivery. But electrical therapy devices (e.g., microelectronics, stretchable electronics, or wireless-connected devices) have become expensive and difficult to use. For example, battery-powered electrical stimulation devices must be connected to electrodes before each use and must be charged between uses. Form factors of conventional devices thus provide a barrier to electrotherapy healthcare adoption and compliance. The cost and encumbrance of electrotherapy contrasts with the “take and forget” usability of tablet pharmaceuticals, which contributes to the significant differences in pharmaceutical and electrotherapy-based healthcare.

### SUMMARY

In some example embodiments, there may be provided a method including a substrate layer, the substrate layer configured to support: a battery array comprising a plurality of battery cells, wherein least a first battery cell is electrically coupled to a second battery cell using a conductive interconnect; an interface layer coupled to the substrate layer, wherein the interface layer is electrically coupled the battery array via a translayer interconnect, the interface layer configured to adhere the substrate layer to a body and to provide electrical stimulation to the body.

In some variations, one or more of the features disclosed herein including the following features can optionally be included in any feasible combination. The first battery cell of the battery array is staggered from the second battery cell of the array such that an axis of bending is present between the first battery cell and the second battery cell. The substrate layer is generated by printing. The first battery cell and the second battery cell are connected in series. The translayer interconnect couples the interface layer and the battery array via a through-hole, wherein the through-hole comprises a perforation of the substrate layer. The through-hole comprises a metallic ink. A battery cell of the plurality of battery cells comprises an anode and a cathode. The anode and the cathode are stacked in a pair. The anode comprises a metal. The metal is zinc. The cathode comprises a metal oxide. The metal oxide is manganese dioxide. The wearable device further comprises a separator membrane between the anode and the cathode. The substrate layer comprises a polymer. The polymer is polyethylene terephthalate (PET). The interface layer comprises an electrode and an ion-conductive buffer. The ion-conductive buffer comprises a hydrogel sheet or a non-woven sponge. The electrode comprises an anode and a cathode. The conductive interconnect comprises a copper track beneath a carbon conductive track, disposed underneath a dielectric layer. The wearable device further comprises a gas channel in fluid communication with a battery cell of the battery array, the gas channel providing a vent to a space outside of the substrate layer.

### BRIEF DESCRIPTION OF THE DRAWINGS

The accompanying drawings, which are incorporated in and constitute a part of this specification, show certain aspects of the subject matter disclosed herein and, together with the description, help explain some of the principles associated with the disclosed implementations. In the drawings,

FIG. 1, schematically illustrates a wearable disposable electrotherapy device, in accordance with some embodiments;

FIG. 2 illustrates a design pipeline enabling wearable disposable electrotherapy, in accordance with some embodiments;

FIG. 3 illustrates wearable disposable electrotherapy manufacturing processes, in accordance with some embodiments;

FIG. 4 illustrates a load characterization for aa wearable disposable electrotherapy device, in accordance with some embodiments;

FIG. 5 illustrates Battery cell and pack design (sizing), validation, and stimulation for a wearable disposable electrotherapy device, in accordance with some embodiments;

FIG. 6 illustrates validation of exemplary wearable disposable electrotherapy self-limited discharge, in accordance with some embodiments;

FIG. 7 illustrates an application specific wearable disposable electrotherapy design, in accordance with some embodiments;

FIG. 8 illustrates several uses for the wearable disposable electrotherapy device;

FIG. 9 illustrates a formation of 3D structure and battery pack functionality of the wearable disposable electrotherapy device, in accordance with some embodiments;

FIG. 10 illustrates a geometry of exemplary wearable disposable electrotherapy device (battery pack structure and interconnects, interfaces, venting system), in accordance with some embodiments;

FIG. 11 illustrates a 3D geometry of exemplary wearable disposable electrotherapy device, in accordance with some embodiments, in accordance with some embodiments;

FIG. 12 illustrates single battery cell designs;

FIG. 13 illustrates subject-wise simulation of exemplary wearable disposable electrotherapy device performance;

FIG. 14 illustrates temperature transition of exemplary wearable disposable electrotherapy device, in accordance with some embodiments;

FIG. 15 illustrates performance of iontophoresis application wearable disposable electrotherapy device on a skin phantom, in accordance with some embodiments;

FIG. 16 shows response functions of the device;

FIG. 17 shows a coupled system;

FIG. 18 shows a numerical implementation of the coupled system;

FIG. 19 illustrates a structure of a battery pack with cross-hatch connector, in accordance with some embodiments;

FIGS. 20A-B illustrate configurations of battery anodes, in accordance with some embodiments;

FIGS. 21A-C, illustrate configurations of battery packs, in accordance with some embodiments;

FIG. 22 shows validated wearable disposable electrotherapy performance with DC-waveform based use-cases;

FIG. 23 illustrates a plan and design pipeline for a wearable disposable electrotherapy device;

FIG. 24 illustrates an end-to-end pipeline for simulations and design of wearable disposable electrotherapy to emulate any conventional device output; and

FIG. 25 illustrates adaptive meshing and computation.

### DETAILED DESCRIPTION

Described is an electrotherapy device that automatically (e.g., when applied to the skin) provides controlled discharge in a single-use, disposable, low-cost, conformable bandage. Enabling these features is an integrated printable design, with power source, self-limiting mechanism, and interface elements made from environmentally benign common materials assembled layer by layer. The entire platform is printed on a common substrate, with an emergent 3D electrochemical architecture that is novel and enabling. In some implementations, the device is made without any circuitry.

The wearable disposable electrotherapy device supports scalability and distribution models akin to pharmaceuticals. Each device can be discreetly carried, applied in any environment (e.g., a situation such as in a hospital, or a doctor's office, or a clinic, or a rehabilitation center, or at work, or at home, or in transport), and disposed after use, like an adhesive bandage. This form factor eases adoption of the device by trained medical personnel. The wearable disposable electrotherapy device can perform transdermal drug-eluting therapy when drugs with charge carriers are incorporated into the device. Here, compliance (e.g., the ability of the patient to intake a prescribed amount of a drug) compared to tablet drugs can be enhanced by continuous transdermal infusion. Almost any existing application of non-invasive electrotherapy can be enhanced by the device, including democratized access to neuromodulation for pain (migraine) and neuropsychiatric disorders. Herein is described the device's robustness for three applications: neuromodulation, accelerated wound healing, and iontophoresis, although the device can be used for many other applications.

Manufacturing the wearable disposable electrotherapy device uses system design processes, a theoretical framework for self-limited dose control, novel battery cell technology, and battery pack architecture adapted to scalable manufacturing processes. Further sections provide information about the device's performance according to the design process.

The wearable disposable electrotherapy device can compete in cost and usability with tablet pharmaceuticals. The design of the wearable disposable electrotherapy device can address interdependent considerations including automatically initiated and controlled dosing, power density, packaging, and scalable manufacturing in a device manufactured with only common, environmentally benign, and nonhazardous materials. The device design supports broad application-specific flexibility.

To improve environmental impacts, the device eschews conventional electronics (e.g. printed circuits, and heavy metals). Instead, the device can control electrotherapy using a printed structure with modular battery cells with interconnects. Together, the three-dimensional (3D) battery pack structure, cell shape (tailoring areal energy density to thickness), active materials, and mass inventory provide the requisite voltage and dose control. Dose ramping is achieved through power-load interface design (internal battery pack resistance, electrodes, hydrogel thickness, and ion mobility) based on the progressive impedance changes associated with device application/removal.

The common (embedded) substrate for all power/interface components can remove any steps by the user (“no assembly required”). Devices can be activated upon contact, where the body completes the device discharge circuit (150). Therefore, to use the device, one needs only to apply it (e.g., absence of any controls, even a start button).

The platform's 3D architecture is manufactured entirely using additive/subtractive fabrication with common/benign materials including active materials based on alkaline Zn/MnO₂ electrochemistry. Moreover, the battery does not require charging, instead, it can be fully activated during fabrication.

Packing, power (maximum current, capacity), shape, and conformability design requirements can be addressed in an iterative design workflow. There is an overlay of electrochemical design, working-temperature regulation, and mechanical design to support needed conformability, and sealing structures (including venting system; 1040, 1130, 1160).

Consequent these features, the disposable wearable electrotherapy device can be distributed and used as economically and simply as pharmaceuticals are.

Achieving these features utilized an integrated design procedure that was demonstrated and validated in detail for an exemplary platform and three electrotherapy applications. Replication of efficacy by the disposable wearable electrotherapy device can require only the imitation of dose, namely current over time and interface shape or position. For each application, the dose can therefore be the design input against which device elements (design output) are verified or validated. The design process can be iterative or driven by design outputs.

Unlike existing electrotherapy devices with electronic output control, the described device overcomes a fundamental challenge for dose control: the nonlinearity of both energy sources (battery pack) and interfaces-physiological loads. unlike existing electrotherapy devices with electronic output control. A design process which can be governed by application-specific design inputs, separately: 1) can characterize interface-biological impedance loads across subjects; and 2) can develop self-regulated battery cells, which can be sized into battery packs. Then, the discharge of a designed battery pack through the interface and biological loads can be simulated. The simulation can support iterative battery/pack optimization to ensure reliable output control across subjects. To simulate the interaction of the independently characterized (uncoupled) subsystems during operation (coupled), a novel isotemporal-trajectory theory is applied.

$${V}_{B} ( {I}_{n} , t \vert {α}_{B} , {e}_{B} ) = {V}_{L} ( {I}_{n} , t \vert {α}_{L} , {e}_{L} ) \qquad ( Eq . 1 )$$

where voltages V_{B} and V_{L} are the independently characterized battery pack and interfaces-physiological load subsystems, respectively; Iₙ is a constant current stimulus; α_{B} and α_{L} are internal parameters of each system; and e_{B} and e_{L} are environmental factors affecting each subsystem.

Manufacturing interconnects can include electrically connecting all device elements across a 3D architecture. Although interconnects, through-holes, battery current collectors, and stimulation electrodes can be fabricated concurrently, they can be functionally (e.g., electrically or electrochemically) distinct.

Interfaces to the physiological load include the stimulation electrode (anode/cathode) and an ion-conductive buffer (e.g., hydrogel sheet or non-woven sponge). Interfaces can be tested for electrochemical capacity on a phantom, and the design can be refined accordingly (e.g., printing vs. electrochemical corrosion vs. electroplating), leading to interfaces-physiological impedance load characterization (uncoupled) using a sourcemeter. Current levels can straddle the application-specific operating range, applied for the targeted duration and charge, with a compliance voltage limit. This selected voltage can critically impact the performance of the final prototype (coupled system), including current ramp-up time and peak, and so battery pack sizing.

Battery cell design can be an iterative process involving architecture, anode and cathode inks, current collectors, separator membrane, and electrolyte. Battery cells with different designs and sizes can undergo a range of verifications (e.g., galvanostatic discharge, shelf life, or electrochemical impedance spectroscopy (EIS)). Galvanostatic current levels (range) match those used for interface-physiological impedance load tests.

As single batteries can have insufficient potential for delivering a dose, a battery pack consisting of a series of batteries can be used. Battery packs can be configured to provide an application-specific amount of energy and a self-limiting mechanism, with an initial (peak) voltage matched to a compliance voltage limit of interfaces-physiological load tests. Battery pack sizing can include selection of the number of battery cells, cell types, and size of cells. Battery packs can be homogenous (e.g., with a single battery cell type/size) or inhomogeneous (e.g., including at least one battery cell that has a different type or size from at least one other battery cell). Using an inhomogeneous can enhance flexibility in tracking the prescribed dose. Sized battery packs can be fabricated and galvanostatically tested using the established current levels (range). Hence, the voltage limit and current levels can be matched for characterization of the two subsystems: the interfaces-physiological load and battery pack.

Given data from the two uncoupled subsystems (battery pack and interfaces-physiological load) the behavior of the coupled system can be simulated using isotemporal-trajectory theory. The numerical solution provided by this theoretical framework can simulate device discharge, which can be compared with dose design inputs to consider modifications to the battery pack in an iterative manner.

These system design processes can yield application-specific battery pack configurations, developed in parallel with sealing techniques, venting system design, and comfortability design, all under rigorous limits on acceptable manufacturing methods (e.g., to manufacture a product that is scalable, economical, and environmentally benign). Final prototypes can be tested against design inputs in human trials to validate each application of the disposable wearable disposable electrotherapy device.

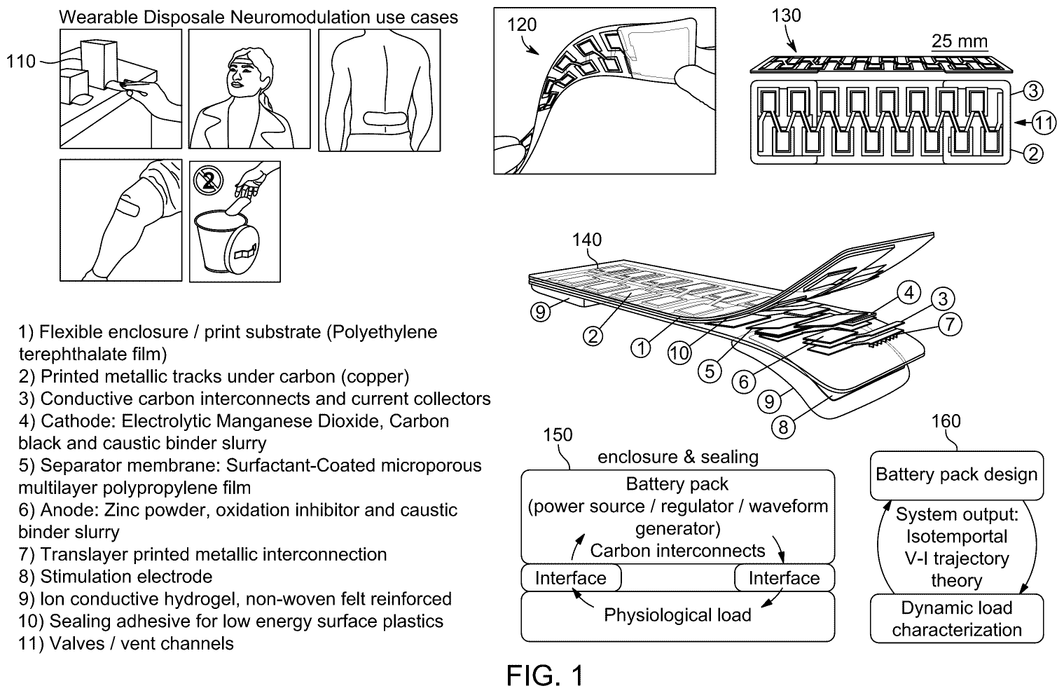

FIG. 1 schematically illustrates a wearable disposable electrotherapy device, in accordance with some embodiments.

The wearable disposable electrotherapy device can be an application-specific single-use device (110) can be distributed and used akin to a disposable bandage or a pharmaceutical. Applications can include brain/cranial nerve stimulation (e.g. to improve cognition or treat a headache), electrical stimulation for pain, and accelerated wound healing. Devices can be disposable as they can be made without conventional electronics, using environmentally-benign materials. 120 shows a photograph of exemplary device.

Device performance enabled by a layered geometry (130, 140) of active materials and interfaces (element types 1-11) printed onto a common substrate, which becomes the device enclosure. 150 illustrates a system diagram: The battery pack discharge can be initiated and governed by interaction, through the interface, with a target physiological load. An application-specific therapeutic dose (e.g. for neuromodulation, transdermal drug delivery, or wound healing) can be thus controlled by the device shape and battery pack design.

Unlike prior neuromodulation or battery technologies, discharge can be neither current nor voltage controlled. Rather, device chemistry and architecture (battery packs) can be designed based on a discharge theory (160).

The wearable disposable electrotherapy device elements (130, 140) include the electrochemical cells connected in series (configuration 930), interconnects and interface elements (stimulation electrodes and hydrogel), sealing and venting system, and skin adhesive or dressing (depending on the application). The design can integrate discharge tuned per application, additive manufacturing and robust sealing, and high conformability.

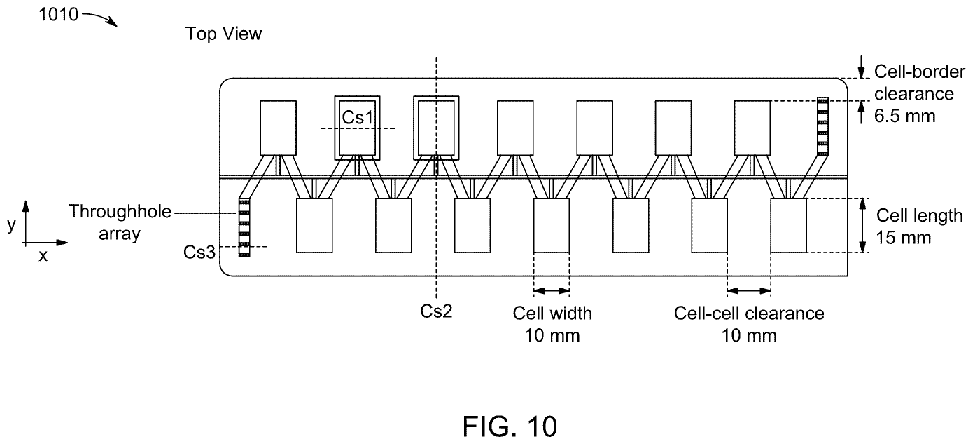

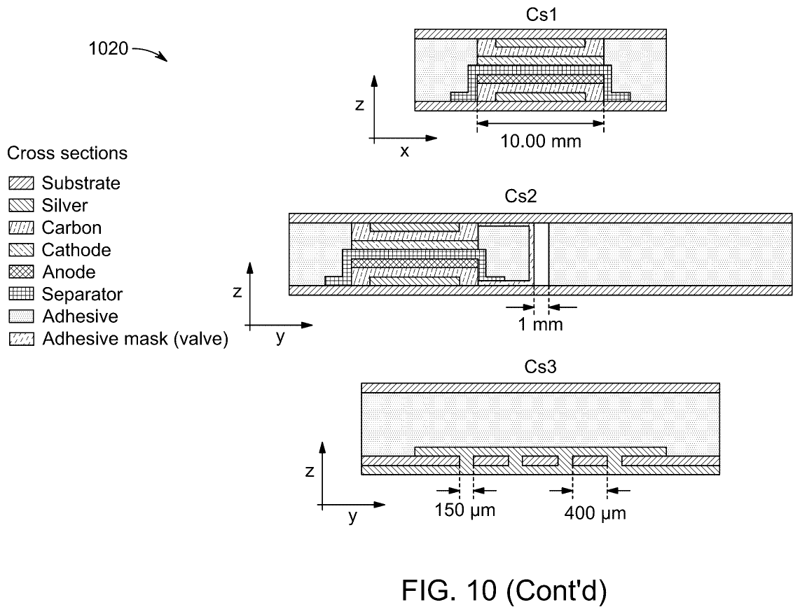

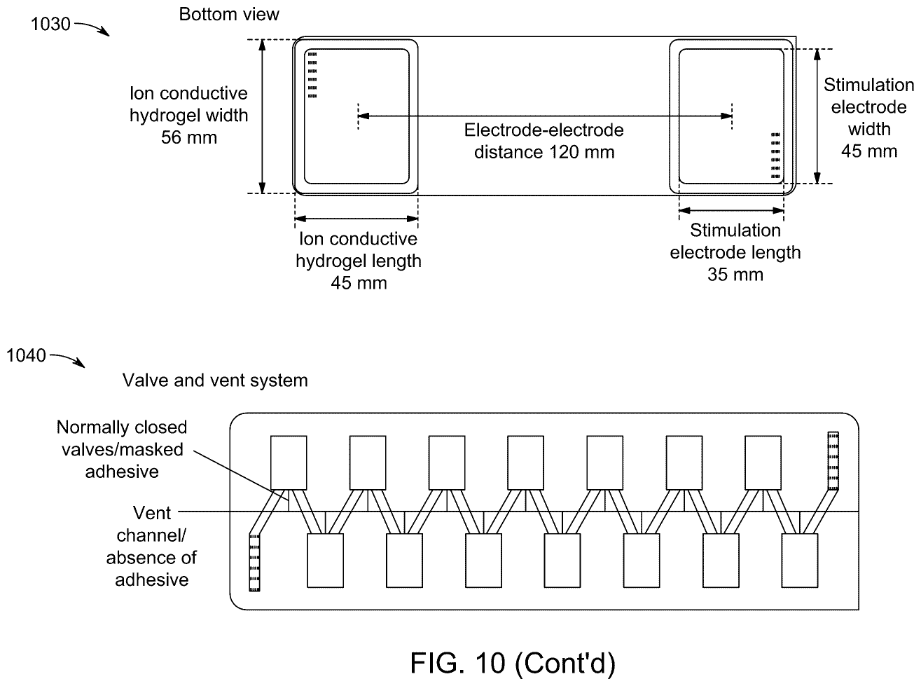

Printed battery cells including an associated anode and cathode, with corresponding cell terminals, can be sealed to prevent electrolyte losses as well as to minimize cell-to-cell parasitic losses resulting from electrolyte sharing between cells (FIG. 10). The anode terminal of a first cell and the cathode terminal of a last cell in series comprise the terminals of the battery pack (power supply). These terminals are connected to the interface, including the stimulation electrode and ion-conductive buffer, which in turn provide connectivity to the body.

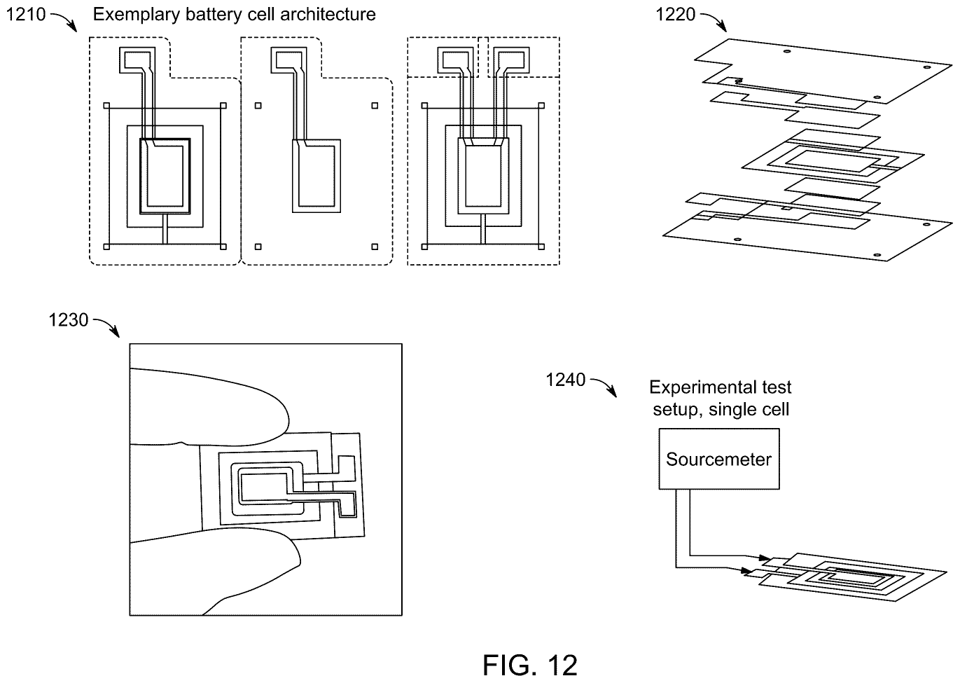

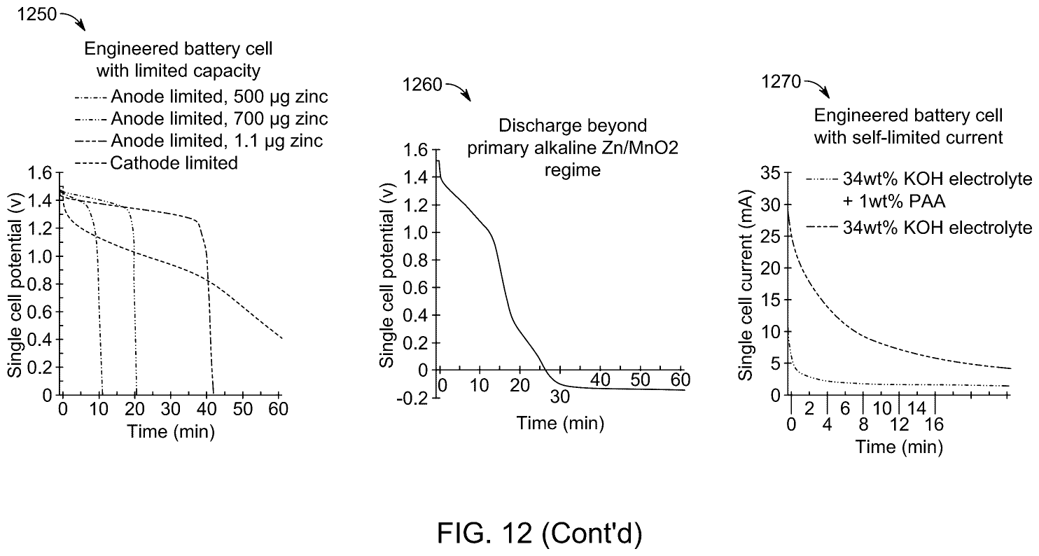

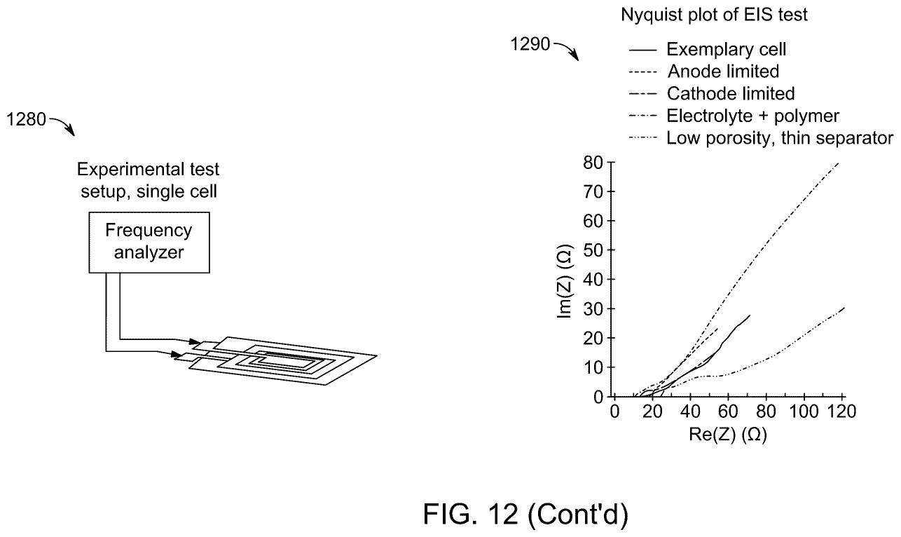

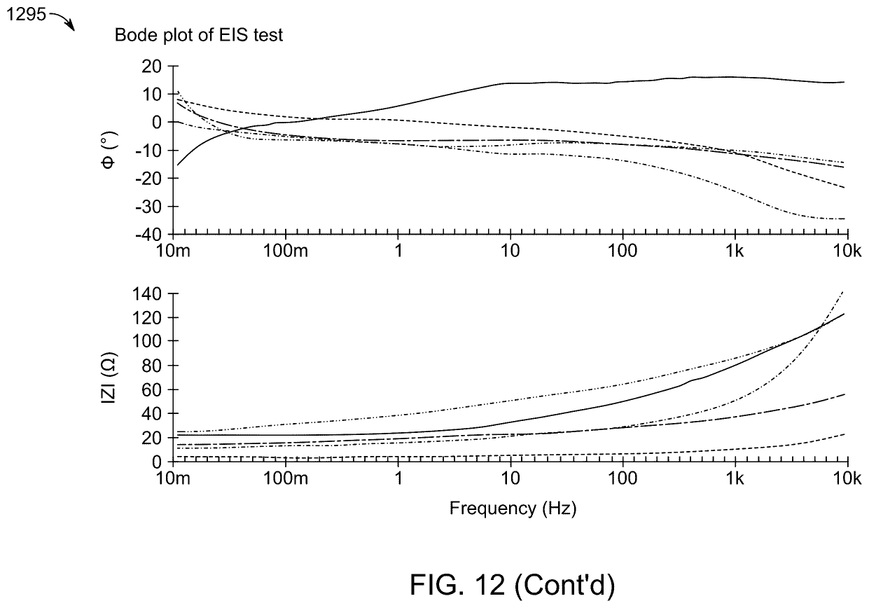

The batteries can adapt primary aqueous alkaline zinc/manganese dioxide chemistry. Electrolytic manganese dioxide (EMD) can be the cathode active material while metallic zinc can be the active anode material (FIG. 12). This chemistry offers safety, high energy density, and a self-limited discharge rate. The anode and cathode can be separated by a specialized membrane made from a porous polypropylene (PP) film laminated to a non-woven PP, coated with a hydrophilic surfactant for aqueous applications. The membrane can be suitable for extreme pH levels, and it can maintain mechanical stability once wet. High device discharge rate can be achieved by this high-porosity membrane, high surface area of each cell, low cell thickness, and high conductivity interconnects. As the application is not steady-state (e.g., because the current can change as a function of time), EIS is a standard tool to characterize the dynamic behavior of varied cell constructs (1280, 1290, 1295), to then inform application specific battery packs. Hydrogen gas generation (which can deform the enclosure) can be lowered using a zinc alloy (e.g., with <100 ppm of indium and bismuth), and corrosion inhibitor additive to the alkaline electrolyte and is managed using a vent system (individual battery vents converging to one central channel of one millimeter (mm) width, 1040, 1130), which also supports effective vacuum sealing. A cell printing and packing approach can fit a required number of cells in series within the physical constraints of the device area and can support manufacturing. Anodes, cathodes, and their connections can be printed in successive steps, for example, in a symmetric geometric fashion on a plastic substrate. These printed sheets can form the device enclosure. The interconnects between anodes and cathodes can produce the required in-series connection between cells. In the final manufacturing step, the sheet can be folded along its symmetry axis, marrying each anode element to its corresponding cathode element (of each cell), resulting in a fully functionalized battery pack (process steps 910).

The disposable wearable electrotherapy device can include regions of relatively high flexural rigidity (battery cells) bisected by axes of low flexural rigidity (920i). This mechanical design, that follows from the planar interlaced battery pack design can support device conformability. Application specific design can further incorporate other design elements for conformability including articulated enclosures pattern cuts, or regions of low flexural rigidity (e.g. interface regions with a single substrate (920iv)).

In one implementation, 20% of the areal surface is a region of relatively high flexural rigidity. In one implementation, 40% of the areal surface is a region of relatively high flexural rigidity. In one implementation, more than 80% of the areal surface is a region of relatively high flexural rigidity. In one implementation the region of low flexural rigidity includes at least 2, preferably at least 6, more preferably at least 10 pattern cuts. The number of pattern cuts can be approximately the number of battery cells. A device with 10 battery cells can have 10, 9, or 11 pattern cuts. In one implementation, the number of pattern cuts is about half the number of cells. A device with 10 battery cells can have 5, 4, or 6 pattern cuts. In some implementations, one pattern cut can be applied between each battery cell. Alternating pattern cuts can be applied across the surface. Pattern cuts can be applied to the region of low flexural rigidity. A pattern cut can cross between a region of high and a region of low flexural rigidity. A pattern cut can have a controlled thickness of less than 1 mm, less than 0.01 mm, or less than 0.0001 mm. The pattern cut thickness can be 90%, 50%, 20%, or 10% of one substrate thickness. The design of the pattern cuts reflects the design configuration of the device on the body.

The substrate material can support sufficient surface adhesion and compatibility with ink solvents and the extreme pH conditions of battery materials, while also providing the required mechanical characteristics for the application. Materials such as polyethylene terephthalate (PET) can be disposable, cost-effective, and environmentally friendly, aligning with the demands of wearable disposable electrotherapy applications.

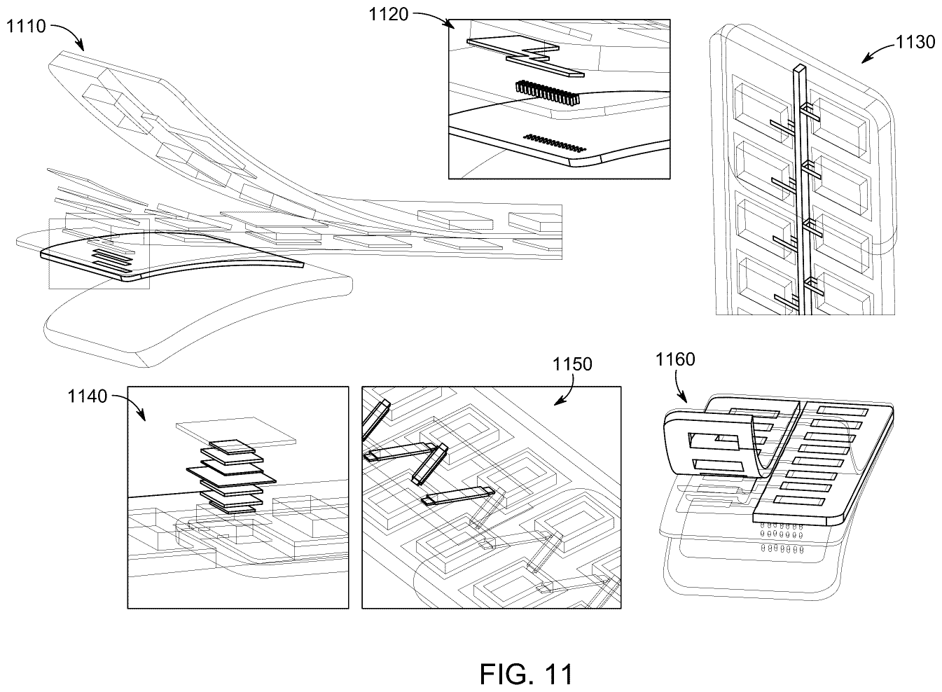

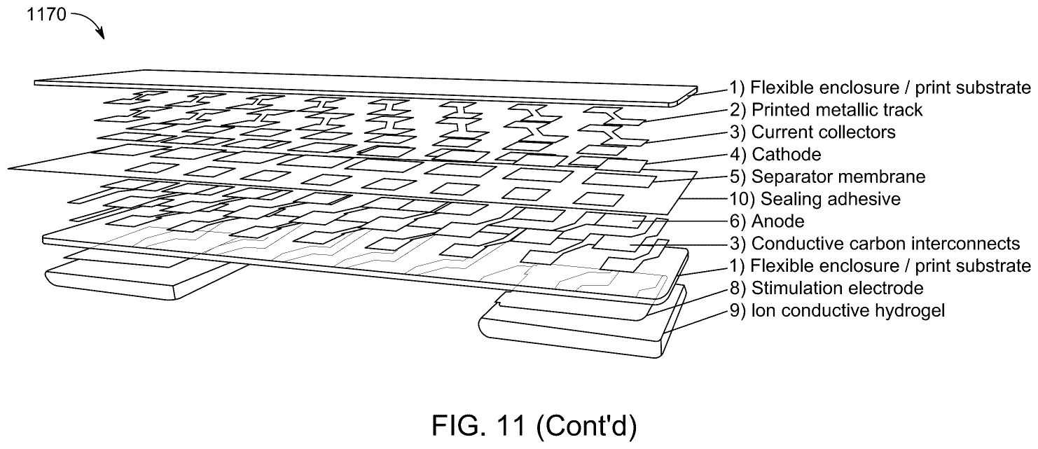

Interconnects can be printed on both sides of the substrate: on the inner side (upon folding) connecting sequential batteries and on the outer side connecting battery pack terminals to the electrode of the interface (herein also referred to as a “translayer interconnect”) (FIG. 10; FIG. 11). An interconnect can include a narrower highly conductive track beneath a wider carbon conductive track, topped by a broader dielectric layer. The highly conductive track can be copper or another suitable metal. This architecture can result in interconnects with 75% greater conductivity. The highly conductive track can be 1×, 2×, 5×, or 10× the width of the interconnect. The highly conductive track can have a thickness of 0.1×, 0.5×, 1×, 5×, or 10× of the interconnect. The number of highly conductive tracks can be approximately the number of battery cells. A device with 10 battery cells can have 11, 10, or nine highly conductive tracks. The number of highly conductive track tracks can be approximately half number of battery cells. A device with 10 battery cells can have six, five, or four highly conductive tracks. A device with four battery cells can have three, two, or one highly conductive tracks. The highly conductive track can have a conductivity of 1×, 10×, 50×, or more than 100× of the interconnect. In one implementation, the highly conductive track is printed on both side of the interconnect.

The device can be coupled to the skin through the interface elements (hydrogels), facilitating electrical charge delivery and physical support of the device on skin. The interface elements (on the exterior of the substrate) can be electrically connected to the battery pack (e.g., inside the pack). Though-holes connections can be arrays of micrometer-sized holes perforating the bottom substrate (1120). For the implementation of through-holes, metallic ink can be used, and the geometry/size (micrometer (um) range) of the holes through the substrate are adjusted based on the rheology of the metallic ink. In one implementation, the total cross section of the through-holes is less than 10%, 5%, or 1% of the hydrogel contact area. Through-hole size (radius) can be less than 0.1 mm, 0.001 mm, or 0.00001 mm. The minimal distance between through-hole can be approximately the thickness of the substrate. The total number of through-holes can be more than 10, 100, or 1000. For a device with two 5×5 cm contact area gels, the number of through-holes can be 20, 100, or 500. Through-holes can be encased in rigid support material. Through-holes can be designed to be punctured. The puncturing can occur because of device exposure to air, bending, placement on the body, or a combination. The design and activation of the through-holes is design according to the theory presented here.

The battery current collector for the anode can be printed using a conductive ink (e.g., copper ink). For the cathode, carbon ink alone or on top of metallic ink can prevent the corrosion of the current collector in contact with the cathode material. For anode stimulation electrodes, zinc ink, and for cathode stimulation electrodes Ag/AgCl ink printed on a copper surface can provide higher capacity stimulation electrodes for DC applications.

An exemplary disposable wearable electrotherapy device can perform low-intensity transdermal stimulation applications with a target dose of 3±one milliamp (mA) direct current (DC) average over 20 min (single-use ˜150 millicoulombs/square centimeter (mC/cm²) capacity) in a conformable packaging with 25 cm² interface electrodes (12 cm inter-electrode distance). A significant design aspect involves ensuring tolerability by limiting current transients at the initiation (τ>1 min) and end of stimulation (managed in conventional electrotherapy equipment with microcontrollers) and instantaneous peak current not to exceed 5.5 mA.

Another exemplary disposable wearable electrotherapy device can perform low-intensity transdermal stimulation applications with a target dose of 1±one milliamp (mA) direct current (DC) average over 20 min (single-use ˜50 millicoulombs/square centimeter (mC/cm²) capacity) in a conformable packaging with 25 cm² interface electrodes (12 cm inter-electrode distance). Another exemplary disposable wearable electrotherapy device can perform low-intensity transdermal stimulation applications with a target dose of 10±one milliamp (mA) direct current (DC) average over 20 min (single-use ˜500 millicoulombs/square centimeter (mC/cm²) capacity). Another exemplary disposable wearable electrotherapy device can perform low-intensity transdermal stimulation applications with a target dose of 1 milliamp (mA) direct current (DC) average over 200 min. Another exemplary disposable wearable electrotherapy device can perform low-intensity transdermal stimulation applications with a target dose of 0.1 milliamp (mA) direct current (DC) average over 500 min. DC output can be converted to AC or pulsed as described. Another exemplary disposable wearable electrotherapy device can perform low-intensity transdermal stimulation applications with a target dose of 2±1 milliamp peak (mA) alternating current (AC) average over 20 min (single-use <0.5 net millicoulombs/square centimeter (mC/cm²) capacity). Another exemplary disposable wearable electrotherapy device can perform low-intensity transdermal stimulation applications with a target dose of 0.1±1 milliamp peak (mA) alternating current (AC) average over 120 min (single-use <0.1 net millicoulombs/square centimeter (mC/cm²) capacity). Pulsed stimulation can use frequencies of 1 Hz to 10000 Hz, with amplitude of 0.1 to 40 mA peak.

In one implementation, the device adapts the peak current to vary according to subjects. Peak current can vary from a value within 0.1 to 20 mA peak, preferably a value within 0.5 to 10 mA peak, still more preferably to a value within 1 to 5 mA. Varying current so subjects provide benefits in tolerability or efficacy. The control can be achieved through the device features described. In one implementation the device adapts the peak current range to vary according to subjects. Peak current can range vary from a range of 0.1 to 20 mA peak, to a range of 0.5 to 10 mA peak, or to a range of 1 to 5 mA. During a session the current varies within the range. For example, for one subject the current can vary from 0.1 to 5 mA during the session. For another subject, current can vary from 1 mA to 3 mA. The described device uses range adjusted stimulation, compared to fixed current used in prior technologies. In one implementation, the electrodes are less than 25 cm² and the current range during the session is 0.1 to 1 mA, over a duration of 30 to 120 minutes. In one implementation, the electrodes are less than 25 cm² in area and the current range during the session is 0.1 to 0.4 mA, over a duration of 120 to 400 minutes. In one implementation, the electrodes are less than 25 cm² in area and the current range during the beginning session is 0.1 to 0.4 mA, over a duration of 120 minutes followed by a current range during the remainder of the session is 0.1 to 0.2 mA, over a duration of 300 minutes. In one implementation, the electrodes are less than 25 cm² and the current range during the beginning session is 1 to 0.4 mA, over a duration of 120 minutes followed by a current range during the remainder of the session is 0.1 to 0.5 mA, over a duration of 200 minutes. In one implementation, the current range during the beginning session is 0.2 to 0.7 mA, over a duration of 30 minutes followed by the current range during the remainder of the session is 0.1 to 0.2 mA, over a duration of 300 minutes. In one implementation, the current range during the beginning session is 0.2 to 0.7 mA, over a duration of 30 minutes followed by the current range during the remainder of the session is 0.1 to 0.01 mA, over a duration of 200 minutes. In one implementation, the current range during the beginning session is 2 to 8 mA, over a duration of 20 minutes followed by the current range during the remainder of the session is 1 to 0.1 mA, over a duration of 100 minutes. The selection of intensity and range can be governed by the desired application or according to mechanisms of action. A high current range followed by a low current range provides a high initiation dose followed by a sustained dose.

Hydrogel electrodes can provide reliable current passage (>0.5 mm thick; volume resistivity of ˜500 Ω·cm; biocompatibility (ISO 10993-5, ISO 10993-10), and provide mechanical adhesion of the device to the skin (moderate skin pull-off adhesion, 20-50 grams (g)/cm; relative high adhesion on the device side, >100 g/cm), with no residue (e.g. felt reinforced) or irritation. Hydrogel electrodes can provide reliable current passage (>1 mm thick; volume resistivity of ˜500 Ω·cm; biocompatibility and provide mechanical adhesion of the device to the skin (moderate skin pull-off adhesion, 10 grams (g)/cm; relative high adhesion on the device side, >100 g/cm). Hydrogel electrodes can provide reliable current passage (>1 mm thick; volume resistivity of ˜50 Ω·cm; biocompatibility and provide mechanical adhesion of the device to the skin (moderate skin pull-off adhesion, 1 gram (g)/cm; relative high adhesion on the device side, >100 g/cm). Hydrogel electrodes can provide reliable current passage with >1 mm thickness and volume resistivity of ˜500 Ω·cm. Hydrogel electrodes can provide reliable current passage with >10 mm thickness and volume resistivity of ˜1000 Ω·cm. The hydrogel resistivity can be adjusted according to tolerability and mechanisms.

The example disposable wearable electrotherapy device (120, 130, 140, FIG. 12) can include printable layers forming a battery pack (e.g., five layers), conductive interconnects (e.g., four layers), a polyethylene terephthalate (PET) sheet enclosure (two layers), sealant (three layers), polypropylene (PP) strips to mask sealant as normally closed valves connecting inner space of batteries to vent channel, and ion-conductive hydrogels. The 3D design can yield a varied number of layers depending on position along device plane—from three at enclosure ends, to nine over internal battery cells, to 11 at conductive hydrogels (not including vents and through-holes) (1020 and 1130).

### Example Device Parameters

Form: The length of the device is 2 to 1000 times the width. In one implementation, the length is 2 to 100 times the width. In a preferred implementation the length is 5 to 10 times the width. The device has bent edges. The angle of the edges is 10 to 60 degrees. The device has a first width in the center that is 2-40 times a second width at the edge. In another implementation, the first width is 4-10 times the second width.

The wearable electrotherapeutic device featuring a power source of printed primary batteries with chemistries other than alkaline, including but not limited to zinc-carbon, lithium primary, and magnesium primary batteries, wherein these batteries can be manufactured using a printing process for the precise deposition of respective battery materials onto a flexible substrate. The disposable electrotherapy device with a flexible and adjustable geometry, designed to conform to various anatomical locations such as joints, limbs, and the spinal area, for targeted delivery of therapeutic current and drugs.

The shape of the device can be an oval. The oval has an aspect ratio of one to 100. The oval shape can be useful because it increases heat dissipation. The oval ratio of 5-30 is combined with a width length ratio of 7-20 to provide improved dissipation. The length can increase with heat.

Moreover, the conductive fabric has the characteristics of bending resistance and stretching resistance, so that the electrode layer can better keep the stable form in the process of repeated use, and the electrode layer is prevented from being pulled and damaged.

Additive Manufacturing and 3D Printing Processes: The battery design can be adaptable to additive manufacturing technologies, including 3D printing. This involves the capability to layer materials in a precise manner to form the battery components, such as the cathode, anode, and electrolyte layers. The printing process should allow for the creation of complex geometries and internal structures that are not possible with traditional manufacturing methods. Linear designs are provided.

### Flexible Substrates

In one implementation, the battery is built on flexible substrates to enable its application in a wide range of products, including wearable devices and foldable electronics.

The substrate material should be durable yet flexible, maintaining its functionality under various physical stress conditions.

Disposability and Environmental Sustainability: A battery array can be manufactured using materials that are biodegradable, non-toxic, or easily recyclable. The manufacturing process itself should minimize environmental impact, using materials and methods that are eco-friendly.

Benign Materials: A battery array can be manufactured materials that are non-toxic, have low environmental impact, and are safe for both the user and the environment. The selection of materials should also consider the entire lifecycle of the battery array, from production to disposal. The printed primary batteries of various chemistries are encapsulated in packaging materials tailored for each battery type, ensuring moisture resistance and biocompatibility for safe use on the skin.

Integrated Circuits and Sensors: A battery array can be configured to integrate with sensors and circuits that are also printed, creating a complete energy and monitoring solution that is compact and efficient.

Customizability and Scalability: A battery array can be customizable to various sizes and shapes, depending on the application. This flexibility is one of the key advantages of using additive manufacturing techniques.

Energy Efficiency and Density: The battery array can deliver a performance comparable to conventional batteries, making it a viable alternative in various applications.

Safety and Reliability: The battery array can pose minimal risk of overheating, leaking, or causing fire hazards, particularly as it uses new manufacturing techniques and materials.

Electrolyte Composition: The electrolyte in these batteries can be based on lithium hydroxide, sodium hydroxide, or potassium hydroxide. The electrolyte could be in a gelled or solid state, capable of facilitating ionic communication between the cathode and anode.

Cathode Material: The cathode can include a non-stoichiometric metal oxide that contains alkali metal and a proton. The oxide might be a non-stoichiometric alkali metal-containing transition metal oxide, potentially having a layered, spinel, or intergrowth crystal structure. The cathode can include materials such as partially delithiated layered lithium cobalt oxide, lithium manganese oxide, or spinel-type lithium manganese oxide.

Anode Material: The anode could comprise materials like zinc or zinc alloy, possibly in fine or particle form, suitable for use in a primary battery.

Conductive Additives: The battery can include conductive additives within the cathode, such as graphite, carbon black, acetylene black, silver powder, carbon fibers, or graphene, enhancing electrical conductivity and overall battery performance.

Separator Design: A separator capable of preventing the diffusion of soluble oxide species from the cathode to the anode, thereby enhancing the battery's stability and life span.

Battery Housing and Configuration: The battery can be presented in a cylindrical housing with a structured arrangement of the cathode, anode, and separator. The housing could be designed to accommodate the specific requirements of a printable battery, considering factors like thickness, flexibility, and durability.

Additional Components: The battery might include other components like current collectors, seals, and terminal caps, linear printed components, designed to support its functionality and efficiency.

Performance Characteristics: The design of the battery can also focus on the battery's discharge performance, energy density, power output, shelf life, and environmental sustainability. The DTIM step 4 and other associated steps provide a design mechanism.

An exemplary formulation is given:

### Battery Chemistries:

Alkaline
High ratio of KOH in electrolyte to lower gassing
zinc air chemistry
Perforation of print substrate for air contact on cathode side
Cover of holes during production with removable stickers to activate battery when
Needed
Covering anode and cathode with one non-woven PP soaked with electrolyte to form a planar battery

### Battery Production Methods:

Wet production (printed electrodes are still wet with electrolyte when sealing)
Electroplating zinc instead of printing zinc ink
Stencil printing of cathode active material
Perforation of print substrate for folding and aligning (lower density of perforation), cutout (higher density of perforation) after print of active material

### Sealing Methods:

Sealing porous PP membrane (or non-woven PP felt as ion bridge between electrodes)
to PP pouch using ultrasonic welding
Double sided tape with inner liner with acrylic adhesive
Sprayed adhesive on print substrates

### Battery Sizing Algorithm:

Decision about battery pack voltage
Matching battery voltage to load voltage under same current for both
Choosing battery number and capacity to finish the stimulation based on dose

### Dosing:

Battery sizing based on average load
Anode limiting batteries to get flatter discharge curve
Microgram control over zinc mass on each cell (electroplating)
One cell with limited capacity to limit the discharge time
One dry cell (not activated) with plated zinc with controlled mass in microgram range, with an electrolyte sac that the user pops to activate the device.
Plural of not activated cells in parallel that user can activate for higher dosing
Printing carbon resistor in series or parallel to battery pack
Trimming the value of printed resistor to specific value
Printing sensor like resistive material (temperature, humidity, . . . )
Printing OFET for limiting current and pulse generation
Gluing chips using conductive glue to battery pack and stimulation electrodes
Multiple doses for each application where user decides about the strength

### Battery Structure:

Normally-closed valves for each cell
Valves end up in a space in the middle of device, preventing leakage to skin
Series of cells to build up the voltage
For stacked batteries with even number of batteries: alternating battery directions to prevent interconnections between layers of pouch
For stacked batteries with odd number of batteries: extending one layer of pouch to get access to conductive layer
Coplanar battery structure with a non-woven felt soaked with electrolyte covering electrodes of battery

### Conductive Tracks:

Carbon conductive under cathode and silver (nickel, copper) under anode
Through-holes for electrically connecting two sides of a plastic layer
Extended substrate for accessing interconnect
Printing of conductive layers on both sides of the print substrate
Printing of dielectric on printed conductive track to cover it or to print another layer of conductive track on it
Printing metallic track under carbon track to passivate metal track and to increase conductivity of carbon track

### Device Design:

Cut lines on print substrate, where there is no battery cell/interconnects with a specific pattern to increase flexibility of the device
Placing non-conductive hydrogel where there is no stimulation electrode with the same thickness to regulate device temperature with skin temperature and for better physical support
On or off switch, tearing conductive track, deforming or sticking conductive tracks
Electrodes for applications (migraine, tDCS, TENS, wound healing)
Concentric electrodes for wound healing
Choosing a particular voltage and impedance for battery and hydrogel electrode, minimizes risk of shock when placing the device on skin and taking it off
Conformability of the device makes it possible to use it on joints
The device can comprise of two or more individual series of batteries connected to separate sets of stimulation electrodes.
The device can comprise multiple series of batteries where they share a node (nodes) to generate multiple intensities for multiple stimulation electrodes in reference to one stimulation electrode.

### Stimulation Electrodes:

Plated zinc or silver or carbon for anode
Silver oxide or carbon for cathode
Hydrogel suitable for DC stimulation
Non-woven PP or PET glued or welded to pouch covering stimulation electrode, with a saline capsule included in package
Stimulation electrode smaller than hydrogel/saline buffer to prevent skin contact.
At least two electrodes, can be 3 or more
Shape of electrodes
Distance between electrodes

### Preparation Wipes:

In the same package in a separate packaging
Non-woven felt soaked with specific amount of:
Formula:
2% SLS to dissolve superficial skin oil
40% ethanol to clean skin oil and dissolve menthol
54.5% water to hydrate skin
1.5% salt to increase ion conductivity (NaCl, KCl)
2% menthol

### Packaging:

Wet lining inside of package to prevent drying of battery pack and hydrogels
Packaging in altered atmosphere to prevent oxygen from entering the package
Multilayer, barrier for humidity and oxygen

### Architecture

One implementation of the electrotherapy device utilizes a power source comprising a network of primary batteries. There can be 1-100 batteries. In another implementation, there are 2-40 batteries. The batteries can be connected in series, in parallel, or a combination of series and parallel connections. In one implementation, the battery chemistry can be alkaline-based. Each of the batteries can be essentially the same in composition and sizing, or at least one battery can be distinct in composition and sizing. In one implementation, the batteries are in series and the same composition and sizing. In one implementation, the batteries are in series and at least one battery in the series has a distinct composition and sizing from the remainder. The battery(s) with distinct composition and sizing can have reduced capacity with respect to current and/or charge. There can be 2-20 batteries of full capacity and 1-5 batteries of limited capacity. In one implementation, 4-18 batteries of full capacity and one battery of limited capacity. The limited capacity battery(s) can have 10% to 90% of the area of the reminder batteries or, in another implementation, 40% to 75% of the area. The limited capacity battery(ies) can have 20% to 90% of the mass of the reminder batteries or, in another implementation, 30% to 80% of the mass. The limited capacity battery(s) can have 20% to 90% of the mass of the reminder batteries and 50% to 85% of the area. With 14 regular capacity batteries and one limited capacity battery, the limited capacity battery can have 40% of the mass of the reminder batteries or 60% of the area. With 10 regular capacity batteries and one limited capacity battery, the limited capacity battery can have 30% of the mass of the reminder batteries or 50% of the area. With eight regular capacity batteries and one limited capacity battery, the limited capacity battery can have 80% of the mass of the reminder batteries or 90% of the area. When placed in series, the overall discharge of the system can be limited in part by the battery(s) with reduced performance. With multiple types of cells, the tailoring of the number of cells and their relative sizes can provide control on discharge when the load can be poorly defined. The specific number of batteries connected in this manner can be chosen to provide the desired electrical potential necessary for effective stimulation. The selection of both the number and size of these batteries can be modified based on the specific application requirements and the pre-selected dosage of electrotherapy to be administered. The selection design can include dynamic tertiary impedance matching (DTIM).

In this implementation, the batteries are manufactured to be anodic limited with anode composed of zinc alloy. The battery active material can be deposited either through a stencil printing or inkjet printing or via an electroplating process. A stencil in this method can be made of passivated corrosion resistant stainless steel in a rotary form with thickness of one mil to 3 mil.

Electroplating of the zinc can be under controlled current between 10 mA to 100 mA and happens in parallel for all current collectors of a row. The temperature of the electroplating solution can be kept in the range of 20° C. to 50° C., a pump in the solution keeps the electrolyte in circulation, the solution can be in turbulence or laminar flow. In the other manufacturing method, the substrate can be vibrated using a piezo actuator in combination of circulating electrolyte.

Following the deposition of the zinc anode, the cathode material can undergo a compaction process.

The active material of the battery can be initially printed in a wet state, incorporating the electrolyte directly into its formulation. The active material can remain wet throughout the manufacturing process.

In another implementation of the electrotherapy device, a distinct approach can be employed for the primary batteries, utilizing zinc-air chemistry. The substrate for the cathode can be perforated.

The size of these perforations can be strategically tailored to align with the specifications of the conductive ink used while the number and distribution of them are specific to chemistry and current delivery of cathode. This design ensures that the perforations remain open and functional even after the application of the conductive ink, thus maintaining the integrity and functionality of the substrate. In this scenario, cathode ink composition includes specific binder that provides physical support for powdered cathode and specific filler that provides required porosity for oxygen penetration.

Once the perforated substrate, which can also serve as the current collector, can be prepared, it can be initially covered with a removable sticker film. This film can play an important role in deposition of cathode material and protecting it from contact with air. Following this, the cathode ink, formulated for optimal performance in zinc-air battery chemistry, can be deposited onto the prepared perforated current collector.

After the deposition of the cathode ink, the entire cell assembly can be sealed to preserve the battery's active material. These types of devices can be packaged in multilayer packaging with aluminum foil filled with nitrogen. This sealing process can be desirable for maintaining the stability and longevity of the battery until it can be ready for activation.

One atypical feature of this implementation can be the activation mechanism of the batteries. The user, by removing the sticker film, exposes the cathode to air. This exposure can be the used to activating the zinc-air batteries.

In scenarios where the electrotherapy dose should support higher discharge rates, or the battery pack can be constrained by a limited usable area a stacked battery structure can be preferred.

The separator in this stacked architecture can be constructed from a surface-treated porous film made of polypropylene (PP) or cellulose.

Conversely, in situations where the power delivery requirement can be less stringent, the device employs a different battery configuration. In this design, both the anode and cathode are deposited onto the same substrate surface, maintaining a distance between them. The device architecture can be developed through layers. The layers can be numbered for reference, for example in sequence from the surface closest to the body and indexed up one for each layer. A component can occupy any one layer or multiple layers or all layers. In one implementation a plurality of anodes and a plurality of cathodes are manufactured so as to occupy the same layer. In one implementation all the anodes and all the cathodes occupy the same layer. Two anodes and ten cathodes can be used in a 10-layer device with all anodes and cathodes occupying at least layer 5. 12 anodes and 12 cathodes can be used in a 10-layer device with all anodes and cathodes occupying at least layer 6. In one implementation, at least one layer can be the substrate surface and the anodes and cathodes are deposited on the adjacent layer.

In a 10-layer device, layer 3 can be the substrate surface and all anodes and all cathodes occupy at least layer 4. In a 13-layer device, layer 4 can be the substrate surface and at least some anodes and at least some cathodes occupy at least layer 4. The minimum distance between any anode and any cathode in the same layer can be greater than 0.001 um or, in another implementation, greater than 0.1 um or, in another implementation, greater than 10 μm. In a device with 10 anodes and 10 cathodes, the minimum distance between any anode and any cathode can be 10 μm. In a device with 12 anodes and 12 cathodes, the minimum distance between any anode and any cathode can be, in some implementations, 30 μm. In a device with 10 anodes and 10 cathodes, the minimum distance between any anode and any cathode can be, in some implementations, 30 μm, and the minimum distance between any anodes can be, in some implementations, 50 μm. The material between anode and cathodes can be electrically resistive. The resistivity of this material can be greater than 0.01 ohm-cm or, in another implementation, greater than one ohm-cm or, in another implementation, greater than 100 ohm-cm. The next resistance between any anode and any cathode can be greater than one ohm or, in another implementation greater than 200 ohm or, in another implementation, greater than 2 k Ohm. Maintaining either a distance, or resistivity, or resistance, or any combination of these can enhance device performance including reducing electrical shunts or device power inefficiency.

In one implementation, a separator material can be used to cover both anode and cathode. The separator material can occupy one or more layers of the device. In one implementation the separator does not occupy overlapping layers with the anodes or cathodes. In a further implementation the anodes and cathodes occupy one or more adjacent layers and the separator occupies one or more adjacent layer, where the most proximal layer of the anodes or cathodes and the most proximal layers of the separator are adjective. In a 10 layer device, the anodes and the cathodes can occupy layers 5 and 6, and the separator can occupy layer 4. In a 12 layer device, the anodes and the cathodes can occupy layers 5 and 6 and 7, and the separator can occupy layers 3 and 4. In a 10 layer device, the anodes and the cathodes can occupy layer 4, and the separator can occupy layers 5 and 6. In the areal dimension the at least one separator will overlap with at least one anode or at least one cathode. In one implementation the number of separators equals the number of anodes. In one implementation the number of separators equals the number of cathodes. In one implementation the number of separators equals half the number of anodes plus the number of cathodes. At least one separator overlaps at least one anode by an area more than 10% of the anode area or, in one implementation, more than 50% of the anode area or, in another implementation, more than 90% of the anode area. For an anode area of one cm² the separator can overlap 0.9 cm². For an anode area of 2 cm² the separator can overlap 1.5 cm². For an anode area of 0.5 cm² the separator can overlap 0.3 cm². At least one separator overlaps with at least one area of at least one anode and at least one area of one cathode. In one implementation one separator overlaps with one anode and one cathode. In one implementation a separator overlaps with each anode and cathode pair. The separator can be a rectangle. The length of the rectangle can be 2 to 100 times the width. In one implementation at least one anode can be one cm², and at least one cathode can be one cm², and at least one separator can be placed so as to overlap with at least one anode and one cathode.

The device can include printed current collectors and conductive layers optimized for each type of primary battery chemistry, using materials that provide efficient electron transport and collection across the different battery types.

A piece of non-woven PP or cellulose film can be used to cover both electrodes. This non-woven film, known for its excellent electrolyte retention and ion conductivity properties, plays a dual role. It can be securely affixed to the substrate, either through an ultrasonic welding process or with the use of a specialized glue, ensuring structural integrity. Secondly, the film acts as a medium for ion exchange. The non-woven film can be soaked with a precise amount of electrolyte. The presence of this ion bridge facilitates electrochemical reactions, enabling the battery to deliver power in a controlled and efficient manner.

In one method of controlling dosing within the electrotherapy device, one or more battery cells manufactured in an electrolyte-free state, while concurrently storing the electrolytic activating agent in a separate compartment.

In one implementation the electrolytic activating agent storage mechanism can be a hermetically sealed, flexible chamber. This chamber can be connected to the battery cell through an opening. To initiate stimulation the user can manually exert pressure on the chamber, causing it to deflate. This action releases the electrolytic activating agent, which then flows into the not-activated battery cell through the predetermined opening. The infusion of the electrolytic activating agent into the previously dry cell triggers the electrochemical reactions necessary for power generation, thus starting the stimulation process. The electrolytic activating agent has a viscosity of less than 50 poise or, in another implementation, less than 10 poise or, in another implementation, less than one poise. The viscosity of the battery cells so manufactured can be greater than 2 poise or, in another implementation, greater than 15 poise or, in another implementation, greater than 60 poise. The ionic strength of the electrolytic activating agent can be greater than 0.01 mM or, in another implementation, greater than 0.1 mM or, in yet another implementation, greater than 10 mM, the ionic strength of the battery cells so made can be less than 20 mM or, in one implementation, less than 5 mM or in another implementation, greater than 0.1 mM. The volume of the electrolytic activating agent can be controlled relative to the volume of the battery cells so manufactured. The volume of the electrolyte can be 0.1 to 50 times the volume of the battery cell. In one implementation the volume of the electrolyte can be 2 times the volume of the battery cell. In one implementation the volume of the electrolyte can be 0.5 times the volume of the battery cell. The electrolytic activating agent storage can be in one or more layers of the layered device. The layers of the electrolytic activating agent storage can overlap with at least some layers of the battery cells so made. The channel connecting the electrolytic activating agent storage and the battery cells so made occupies at least some of these overlapping layers. In one implementation of the multilayer device at least one layer includes at least one battery cell, at least one electrolytic activating agent storage, and at least one connector. The connector can function as a gate. When the device can be compressed or bent the connector changes from a closed state. In one implementation the change can be irreversible resulting in the discharge of the device. The electrolytic activating agent can be activated by a mechanical adjusted linear printed component or other linear printed component as described. This design allows single use control.

Further enhancing this design can be the possibility of implementing this mechanism across multiple battery cells configured in parallel. By popping and activating separate electrolyte sacs, the total capacity of limiting cells will increase and a greater amount of power can be generated, allowing the device to deliver more intensive stimulation when needed.

In an alternative implementation, the ionic salt can be added to the cathode material, and the sac can be filled with water, upon contact of water to cathode material, electrolyte forms which activate the battery. This implementation makes the device safer for use by not storing extreme pH solutions.

Another approach for dose control within the printed electrotherapy device can be integration of linear printed components with control conductivity. The linear printed components conductivity can be low so that the components are like resistors. Linear printed components can be made from lower conductivity carbon ink. In one implementation at least one of the linear printed components has a area less than 10 mm², preferably less than one mm², still more preferably less than 0.1 mm². In one implementation the length of at least one of the linear printed components can be more than 10 times the width, preferably more than 20 times the width, still more preferably 150 times the width. There can be one to 500 printed linear components as supporting device performance. Printed linear components can be in series or in parallel with other printed linear components. Printed linear components can be in series or in parallel with battery packs. In one implementation the battery packs are in series with each other and with at least one printed linear component. The printed linear component has a resistance greater than 0.01 ohm or, in one implementation, greater than one ohm or, in another implementation, greater than 20 ohm. In one implementation the printed linear component and battery packs are in series and the resistance of the printed linear component can be 10 times the internal resistance of each battery pack. In one implementation the printed linear component and battery packs are in series and the resistance of the printed linear component can be 20 times the combined internal resistance of the battery packs. 20 battery packs in series can be used in series with a printed linear component where the total open circuit voltage of the battery packs can be greater than 15 volts and the resistance of the printed linear component can be greater than 100 ohms. 15 battery packs in series can be used in series with a printed linear component where the total open circuit voltage of the battery packs can be greater than 20 volts and the resistance of the printed linear component can be greater than 200 ohms. 12 battery packs in series can be used in series with a printed linear component where the total open circuit voltage of the battery packs can be greater than 15 volts and the resistance of the printed linear component can be greater than 150 ohms. The control of battery pack design and printed linear component can regulate device discharges. The linear printed components can have constant resistance value or resistance value that changes by the device design. The linear printed components can be sensitive to environmental changes such as temperature and humidity. The design sensitivity enables the linear printed components to dynamically adjust their resistance in response to external conditions, for tuning the stimulation dose. Temperature adjusted linear printed components can be designed according to heat control elements manufactured as described. In one implementation a temperature increase of 3° C., reduces the resistance of at least one linear printed component by more than 10% or, in one implementation, more than 20% or, in yet another implementation more than 50%. In one implementation a temperature increase of 5° C. reduces the resistance of at least one linear printed component by more than 15% or, in one implementation, more than 25% or, in another implementation, more than 30%. In one implementation a temperature increase of 3° C., reduces the resistance of at least one linear printed component by more than 5 ohm or, in one implementation, more than 20 ohm or, in another implementation, more than 40 ohm. In one implementation a temperature increase of 6° C., reduces the resistance of at least one linear printed component by more than 10 ohm or, in one implementation, more than 15 ohm or, in another implementation, more than 35 ohm. When the temperature adjusted linear printed component can be in series with the battery cells, output can be increased. When the temperature adjusted linear printed component can be in parallel with the battery cells, output can be decreased. In one implementation at least two temperature adjusted linear printed components, with one in series and the other in parallel with at least one of the battery elements. In a further implementation with one in series and the other in parallel with all of the battery elements. The temperature adjusted linear printed component can be within 10 mm of the interior surface of the enclosure or, in one implementation, within 5 mm or, in yet another implementation, within 2 mm.

The temperature adjusted linear printed components can undergo a category state change that results in a new device mode. Changes in dose can be classified as device modes. Device mode features include intensity, frequency, and pulse width. Frequency can be one Hz, 150 Hz, 1000 Hz, or 500 Hz. Pulse width can be 1 s, 0.1 s, 0.01 s, or 0.0005 s. In one implementation the mode has a current of less than one mA or, in one implementation, less than 0.1 mA or, in another implementation, less than 0.05 mA. The temperature adjusted linear printed component can be in series with at least one of the device electrodes or in series with at least one of the device battery cells. The temperature adjusted linear printed component changes resistance to above 100 ohm or, in one implementation, above 1000 ohm or, in another implementation, above 10,000 ohm. This can be associated with a low current model. The low current model can be initiated after 5 minutes, 15 minutes, 20 minutes, or 60 minutes of device application to the skin. The low current model can be initiated through heat control elements controlling device output. The change can be linear printed component resistance can be irreversible. An irreversible change produces a limited use or single use device. Similarly a low power or frequency mode can be initiated through heat control elements. A temperature adjusted linear printed component has a cross-sectional area in at least one portion of less than 0.1 mm² or, in one implementation, less than 0.01 mm² or, in another implementation, less than 0.001 mm². In one implementation two or more temperature adjusted linear printed components are placed in series. The change in each element leads to a new mode. Three such elements can produce three more. Ten such elements can produce 10 modes. The temperature adjusted linear printed components used to change mode are surrounded over more than 50% of its surface with an insulative material or, in one implementation, more than 75% or, in another implementation, more than 95%. The insulative material has a resistivity 10 times greater than the temperature adjusted linear printed component or, in one implementation, 50 times greater or, in another implementation, 300 times greater. This enhances mode switching. The temperature adjusted linear printed component changes resistance to above 0.001 uF, preferably above 0.01 uF, still more preferably above 0.5 uF.

The mechanical adjusted linear printed components can be configured according to the circuit and dimensional factors indicated but are especially sensitive to force, pressure, bending, sheer, sliding, stretch, motion, or other mechanical signals as coupled to the peculiar design elements of the device. The mechanical adjusted linear printed components can modulate device function, modes, initiate, or terminate stimulation. The enclosure of the device can be modified with an electromechanical actuator. The electromechanical actuator can be over the surface of the enclosure, embedded in the enclosure, or under the enclosure. The position of the electromechanical actuator can be identified through marker on the enclosure, by a deformation of the enclosure, by the enclosure transparency, or a combination. The electromechanical actuator can be integrated with the architectural layers of the device. The electromechanical actuator occupies at least one layer of the device. The electromechanical actuator can occupy multiple layers where at least one of the layers can be also occupied by at least part of the enclosure. The electromechanical actuator can be coupled to the battery cells or the electrodes or both. In one implementation, the electromechanical actuator has at least two states where each state can be associated with at least one device mode. The function of the electromechanical actuator can be actualized through interconnects. At least one interconnect occupies at least one layer occupied by at least one electromechanical actuator. At least one interconnect can be in electrical continuity with an electromechanical actuator. In one implementation the interconnect occupies at least two layers where one of those layers can be occupied by an associated electromechanical actuator and one of the layers can be not occupied by an associated electromechanical actuator. In a further implementation, the layer not occupied by an associated electromechanical actuator can be occupied by at least a battery cell. The electromechanical actuator can be in series with at least one battery cell or at least one electrode. In one implementation the electromechanical actuator can be in series with at least one electrode and at least one battery cell. The mechanical signal applied to the electromechanical actuator changes its state. In one implementation the electromechanical actuator has three states. In a further implementation the three states are associated with changes in the output current of the device. The output current changes by 1.5 to 3 folds between states.

The output can be 5 mA in one state, 3 mA can be a second state, and one mA in a third state. The output can be 10 mA in one state, 8 mA can be a second state, and 2 mA in a third state. The output can be 4 mA in one state, 2 mA can be a second state, and 0.1 mA in a third state. The output can be 6 mA in one state, 4 mA can be a second state, and 2 mA in a third state. The mechanical adjusted linear changes resistance by 20 ohm, 100 ohm, or 1000 ohm between states. The mechanical adjusted linear changes resistance by a factor of 2, 14, or 100 between states. A change in state can be associated with a decrease of the minimum axial area of the mechanical adjusted linear by 10% or, in one implementation, by 50% or, in another implementation, by 90%. The electromechanical can change its presence in the layer with the mechanical signal. In one implementation there can be at least one layer in which in one state the electromechanical actuator can be not present and where the electromechanical actuator can be present in the layer in a second state. In a further implementation that layer includes an interconnect. In one implementation the electromechanical actuator can be integrated with the electrolytic activation agent described. In one implementation the mechanical adjusted linear element connects with three layers in one mode. In one implementation the mechanical adjusted linear element connects with four layers in one mode.

In one implementation at least one mechanical adjusted linear element can be also a temperature adjusted linear element. includes visual or tactile indicators to confirm battery activation and therapy initiation, providing assurance to the user that the device can be functioning correctly upon application. The device can be activated upon skin contact, which can include mechanisms of mechanical signals such ss pressure-sensitive switches, conductive elements that complete a circuit upon contact with skin, or moisture-activated components. The mechanical components can provide a sound such as a click on state change. The mechanical components can change color, for example to green, to yellow, or to red, on state change.

The disclosure provides for the user experience or dose workflow for reliable or tolerated stimulation. In one implementation, the dose workflow includes up to five stages. The stages can be combined in various orders. Some stages can be omitted. Some stages can be repeated.

The stages are implemented according to the application, device substrate layered architecture, and user needs.

The device exposure stage includes aspects of preparing the device enclosure, electrode, or dynamic linear components. The device exposure stage changes the environment of the device.

The device application stage includes aspects of positioning the device on the tissue or body.

The device can be shaped according to the position on the body. The device can be bent, rectangle, circular, square, or a combination of these shapes.

The device actuation stage includes aspects of device settings including mechanical adjusting linear components. The device or its battery components activation mechanism can be integrated with safety features to prevent accidental activation, such as a removable insulating tab or a switch that uses deliberate action by the user. The device or its battery components can be designed with a specialized control circuit that monitors the connection to the skin and automatically initiates the therapeutic current when a secure attachment can be detected. In some implementations, the therapeutic current can be initiated before contact with the skin (e.g., using a button press or by peeling a sticker off of the interface).

The device discharge stage corresponds to the significant portion of discharge into the body or tissue. The battery's long shelf life and activation mechanism are combined with a dosage control system, ensuring that the therapeutic compounds are delivered at the correct rate and duration once the device can be activated.

The device removal stage includes aspects of the workflow related to removing at least some device components from the body or tissue.

In one implementation the workflow can be device exposure stage, then device application stage, then device actuation stage, then device discharge stage, then device removal stage. In one implementation the workflow can be device exposure stage, then device application stage, then device actuation stage, then device discharge stage, then device actuation stage, then device discharge stage, then device removal stage. In one implementation the workflow can be device exposure stage, then device application stage, then device discharge stage, then device actuation stage, then device removal stage. In one implementation the workflow can be device exposure stage, then device actuation stage, then device application stage, then device discharge stage, then device removal stage.

To achieve precision in value of linear printed component elements or printed resistor material, an adjustment process can be employed during the manufacturing stage. This process involves the use of reduction techniques, wherein the resistance value can be fine-tuned to a specific target. Two primary methods to trim the printed resistive elements after the printing step on the substrate are utilized for this adjustment: physical scraping and laser removal.

Physical Scraping: This method involves automated partial removal of printed resistor material, thereby altering its resistance value.

Laser Removal: In this method, a laser can be employed to partially remove the printed resistor.

One method of implementing safeguard for this device can be the use of printed carbon-based field-effect semiconductive components in conjunction with printed trimmed resistive elements. The specific trimming and measurement after printing step can be for accurate control of current flow.

This method allows implementation of a stimulation current limiting section. By doing so, it effectively limits the energy delivered during therapy, ensuring that the stimulation remains within safe and therapeutic bounds.

By adding to cell number in the battery pack, and incorporating this current limiting technique, the device can regulate and control the dosing of stimulation instead of only acting as a safeguard mechanism. The stimulation intensity directly can be controlled by this current limiting section.

In another use case within the electrotherapy device, printed carbon-based semiconductive components are employed to generate an oscillating signal. The frequency of this section can be preset during the manufacturing process. It also can be changing and interactive. To augment the functionality of this oscillating signal, the device incorporates printed resistive elements that are sensitive to external stimuli, such as pressure, temperature, or humidity.

These resistive components can be designed to dynamically alter their resistance in response to changes in the physical condition of the subject or the surrounding environment. The interaction between the oscillating signal and these responsive resistive elements forms an interactive circuit within the device. The oscillation of this section can be between 0.01 Hz and 1000 kHz or, in one implementation, between 0.1 Hz to 10 kHz or, in yet another implementation, between one Hz to 2 KHz. In one implementation the device output can be 100 Hz 2 mA intensity. In one implementation the device output can be 200 Hz 5 mA intensity. In one implementation the device output can be 1000 Hz one mA intensity. In one implementation the device output can be 10000 Hz 3 mA intensity. In one implementation the device output can be 0.1 Hz 1 mA intensity. The oscillating signal can be heat controlled.

In one implementation of the device, one or more electronic components are placed on the printed substrate and pins are connected to conductive tracks using conductive glue. The combination of printed battery packs with electronic parts that are glued to printed interconnects can be the manufacturing method.

To release the buildup pressure inside of the battery pouch during storage or usage of device, each cell can be equipped with a pressure-activated, normally-closed valve that connects the inside of the cell to outside, allowing release of generated gas. This special valve can be formed by covering or masking a narrow part of the sealant around the battery. This masked part can cover 0.1% to 80% of the side of the battery or, in one implementation, from 1% to 75% or, in another implementation, from 5% to 20%. In one implementation the mast part covers 15% of the side of the battery. In one implementation the mast part covers 20% of the side of the battery. In one implementation the mast part covers 35% of the side of the battery. In one implementation the mast part covers 20% of the side of the battery. Masking of the sealant can be done by printing a non-stick material on the sealant, or on the substrate. This non-stick coat can be printed on the electrical interconnect of the battery on either anode or cathode or both electrodes or it can be on the periphery of the cell where there can be no interconnect. The other method of implementing this pressure relief valve can be not welding a part of the periphery of the cell. In one implementation, the interconnect can be connected to the battery cell.

These normally-closed pressure valves relieve the pressure outside. This area can be shared between cells of the battery pack, can be covered with plastic sheet from bottom and top printing substrates or can be covered with a separate film.

This cover can be a safety mechanism, separating the battery from skin.

One method of implementation of battery packs with stacked-structure batteries can be alternating the direction of adjacent connected batteries to keep the interconnect on the same printing substrate. In this case, every other battery cell can be positioned next to each other to form a row for anodes and a row for cathodes. In one implementation interconnects from all anodes to cathodes are in the same layer. In one implementation interconnects from all anodes to cathodes are in two layers.

The other method of manufacturing battery packs with coplanar-structure batteries, interconnects are routed in “s” form to connect adjacent cells, where anodes and cathodes form two straight rows to improve printability of battery packs.

One of two substrates that form the battery pack can be longer than the other (extended substrate). The conductive track can be printed on the longer substrate, after sealing of the shorter substrate, there can be another printing step that covers the exposed track and extends it to the outside surface of shorter substrate, hence providing an electrical connection from inside of patch to outside to form a stimulation electrode. In the battery pack with stacked structure batteries, if the number of batteries is even, the connection between battery pack and stimulation electrodes can be via through-holes or it can be implemented by “extended substrate” and print of conductive ink to cover the conductive track on extended substrate and can continue on the shorter substrate.

If the number of batteries can be odd, the battery pack can be connected to stimulation electrodes via through-holes or “extended substrate” that can be folded back.

The conductive layer under the battery cathode which forms the cathode current collector, can be composed from 1) printed metallic track and 2) printed conductive carbon layer on top of it.

The metallic track can be removed from the design, or it can be printed narrow under the carbon conductive paint. This metallic track can be composed of silver, copper, or nickel flakes or combination of these metals.

The form of narrower metallic track can be like carbon print (e.g. at least one mm narrower from each side) or it can have a fork shape, parallel narrow lines connected to each other through a narrow metallic track. It can also be in the form of a hollow narrow frame under the carbon print.

Where there can be a need for greater flexibility, the printing substrate will have cuts in form of line or curve, where there are no battery, control, or interconnection elements. These cuts can be in the form of rows close to each other (1 mm to 10 mm distance between neighboring rows), where the neighboring cuts have overlapping rows of cuts and the distance between two cuts in the same line or curve can be between 1 mm to 10 mm. These cuts can also be implemented in double sided tape with carrier film. This approach makes the non-stretchable substrate flexible and conformable.

The bottom of the device, where the device can be placed on the skin and there are no stimulation electrodes, can be covered with non-conductive hydrogel. This non-conductive hydrogel can be used to physically support the device by sticking to the skin. This non-conductive hydrogel can be used to regulate the temperature of printed batteries by heating the thin device to skin temperature through heat conduction. This non-conductive hydrogel can be used to cool off the skin. In this case the backing of the hydrogel can be a wettable material that eases the evaporation of water content of the hydrogel during usage. This non-conductive hydrogel can be infused with active ingredients, such as pharmaceuticals or materials that induce sensations of coolness or heat. This non-conductive hydrogel can cover all around the stimulation electrode or it can cover the area in between them. There can be a distance between non-conductive hydrogel and stimulation electrode. The batteries include separators made from materials like polypropylene or polyethylene, printed as thin layers to enable ionic flow and prevent short-circuiting, contributing to the device's flexibility, thinness, and suitability for wearable applications including transdermal drug delivery or pain management.

In some devices, a part of the conductive track can be printed on a removable plastic tongue that can be placed on the printing substrate before printing step. The user can pull and remove this tongue to disconnect the printed connection when needed. This track can be in parallel with other tracks to lower the stimulation dose.

In some devices, two or more tracks that are printed on two overlapping substrates, with an overlap length are separated with a plastic film in form of a tongue that can be placed between these two tracks during manufacturing. Another plastic piece presses two printed substrates against each other with the removable tongue in between. The user removes the tongue to establish the electrical connection, to turn the device on, or to increase the stimulation intensity. These two techniques can be combined to provide control over device energy delivery.

In one implementation the device can be fabricated through customized additive manufacturing. The process results in a device architecture that can be described in layers. There can be 2-400 layers, preferably 4 to 50 layers or, in one implementation, from 6 to 20 layers. The layers are in cross-section. The number of layers can be the same across the entire device arial plane.

Some components can occupy more than one layer. A component can occupy between one and all layers. Across the areal cross section the number of index layers can remain the same but the composition of each across section can vary. The total number of used stacking arrangements across the device can be 2 to 140 or, in one implementation, from 3 to 40 or, in another implementation, from 4 to 10. The stacking arrangement indicates what occupies each possible layer. The layer can have the same or different thickness. The elements include separators made from materials like polypropylene or polyethylene, printed as thin layers to enable ionic flow and prevent short-circuiting, contributing to the device's flexibility, thinness, and suitability for wearable applications including transdermal drug delivery or pain management.

The dosing of devices can be developed through dynamic tertiary impedance matching (DTIM).

DTIM can be applied in the device design stage or the device quality control stage or the device fighting stage, or any combination of these stages. The electrochemical and architectural device uses DTIM in an application specific manner.

In one implementation, DTIM has five steps. These steps can be varied in order. Steps can be combined into one net step. Specific steps can be skipped or avoided per the application. Each step can be conducted once or repeated.

In the first DTIM step the application can be identified including the target dose. One application can be transdermal stimulation with a target current and target time. The target current can be 0.01 mA to 100 mA and preferably 0.1 mA to 10 mA. The target time can be 10 seconds to 300 minutes, or, in one implementation, from 30 seconds to 60 minutes. One application can be transdermal stimulation with a target current and target time. The target current can be one mA to 10 mA, with a frequency of 100-500 Hz. The target time can be 10 seconds to 300 minutes or, in one implementation, from 30 seconds to 60 minutes. The target can be specified as range. The current intensity range can be 0.01 to 100 mA or, in one implementation, from 0.1 to 50 mA. The target can be specified in voltage. The target can be specified in charge. The target changes can be 0.01 mC to 10000 mC. The charge range can be 10 mC to 40 mC. The charge range can be 150 mC to 400 mC. The target can be specified as a combination of these factors or as a combination of ranges. The placement of the device includes its application specific location. This analysis can be combined with the determination of modified pulse with and intensity as described.

The location can include a surface of the body or any orifice. The size of contact can be 0.1 cm² to 700 cm² or, in one implementation, from one cm² to 50 cm². The surface can include a joint.

The target can be described based on the presentation of symptoms. The device can be placed or impaired skin for example for skin healing or skin improvement. The device can be placed over a painful region. Electrodes contact area can be selected. The number of electrodes can be selected. There can be two electrodes, each electrode 10 cm². There can be five electrodes, one electrode one cm² and four electrodes 5 cm². The electrode material can be selected. More than one dose or electrode can be selected for evaluation in DTIM. The target dose can include a ramp up. A ramp up can be based on intensity. The ramp can be 0.1-1000 seconds from no intensity to more than 90% of the target intensity or, in one implementation, 0.5-120 seconds or, in another implementation, 5-100 seconds. The ramp can be 0.1-1000 seconds from no intensity to more than 80% of the target intensity or, in one implementation, 0.5-100 seconds, or, in another implementation, 10-100 seconds. The ramp can be 1-1000 seconds from no intensity to 70-90% of the target intensity or, in one implementation, 4-200 seconds or, in another implementation, 10-100 seconds. The target dose can include a ramp down. The ramp-down can be specified similar to the ramp but with a disease in intensity. The target dose can be specified with a limit on transient. The target dose can specify no transient 1.1-100× greater than the target dose. The target dose can specify no transient current 1.5-100× of the target peak current. The target dose can specify no transient current for greater than 10-1000 us at 2-200× of the target peak current. Transients can decrease reliability or compromise tolerability. There can be additional discharge limits related to the target dose such as maximum voltage. The maximum voltage can be 10-200 V, preferably 20-60 V. If more than one model or mode are desired, multiple target doses can be defined.

In the second DTIM step, a dynamic probing dose can be applied according to the target dose. If the application of stimulation of the forehead with 2 mA for 20 minutes then approximately 2 mA can be applied for 20 minutes to the forehead. The dynamic probing dose can be provided by a separate or dedicated device. The dynamic probing dose device can be a current controlled source, a voltage controlled source, a source-meter, a battery, or a digital-to-analog converter.

The dynamic probing dose can be any combination of these. The dynamic probing dose device can be configured to provide approximately the test dose. The dynamic probing dose device can be further configured to measure the tissue electrode functional impedance of the system. The functional impedance includes the relevant aspects of electrical impedance, resistance, capacitance, permittivity, or permeability during the application of the stimulation that approximate the test dose. In one implementation a test dose of approximately 4 mA, for 20 minutes. with 100 Hz pulses, biphasic pulse, pulse duration of 100 us can be applied, and the RMS and the peak voltage generated during each pulse can be recorded. In one implementation a test dose of approximately 40 mA, for 10 minutes. with 100 Hz pulses, biphasic pulse, pulse duration of 100 us can be applied, and the RMS and the peak voltage generated during each pulse can be recorded. The dynamic probing dose device provides stimulation over time and the functional impedance of the system can be measured over time. For a 20 minute test dose, measurements can be made at one minute, 5 minutes, 10 minutes, and/or 20 minutes. In one implementation the dose delivered by the dynamic probing dose device can be distinct from the target current in time, intensity, waveform, or any combination of these. For a target dose of eight mA, for 10 minutes, with 50 Hz pulses, biphasic pulse, pulse duration of 100 us the dynamic probing dose device can provide 0.1 mA, for 30 seconds with 50 Hz pulses, biphasic pulse, pulse duration of 100 us. For a target dose of 30 mA, for 60 minutes. with 1 Hz pulses, monophasic pulse, pulse duration of 100 us the dynamic probing dose device can provide 10 mA, for 30 seconds with 100 Hz pulses, biphasic pulse, pulse duration of 200 us. For a target dose of 5 mA, DC, for 30 minutes, the dynamic probing dose device can provide 0.1 mA DC for 60 seconds. Data from the dynamic probing device can be displayed on the device, collected by a A/D, or otherwise recorded for DTIM analysis.

The dynamic probing dose device can provide an incremental dose test. The tissue electrode functional impedance of the system can be accessed by applying 2 to 100 steps of current intensity or, in one implementation, 5-20 steps of current intensity. The current intensity steps can be 0.1 mA, one mA, and 10 mA. The current intensity steps can be 0.01 mA, 0.03 mA, and 0.05 mA. The current intensity steps can be 10 mA, 5 mA, and 20 mA. The tissue electrode functional impedance of the system can be accessed by steps of frequency. The frequency steps can be one Hz, 100 Hz, and 1000 Hz. The frequency steps can be 10 Hz, 100 Hz, and 2000 Hz. The frequency steps can be 0.1 Hz, one Hz, 10 Hz and 20 Hz. During current controlled stimulation the voltage at the dynamic probing dose device output can be sampled at 0.1 to 1000 kHz or, in one implementation, at one to 10000 Hz. The dynamic probing dose device can be coupled to the body using the electrodes selected in the first DTIM step. The dynamic probing dose can be applied in one individual or to a set of individuals. If provided to a set of individuals the tissue electrode functional impedance of the system can be a tissue electrode functional impedance set of the system. The tissue electrode functional impedance set of the system can be from 2-1000 individuals or, in one implementation, 5 to 50 individuals. The set can be divided by subject demographics such as race, sex, age, symptoms, disease diagnosis, skin features or any combination of these. The dynamic probing dose can include additional measurements.

Additional measurements can include functional imaging, fMRI, histology, photography, IR imaging, or questionnaire provided to subjects. Questionnaires can rank pain or discomfort. A VAS assessment can be used. The display can be color coded. The measurements can be analog. Depending on the impedance measure a different color can appear on the device. For example, red for high impedance and green for low impedance. A high impedance can be 10-100 K Ohm or above. A low impedance can be 0.1 to 10 K Ohm.

In the third DTIM step, the electrochemical or discharge performance of device test cells are determined. The device used in test cells can be single battery cells. The battery cells can have parameters systematically varied including chemical composition, size, volume, areal density, or number. This analysis yielded the device configuration functional impedance. The devices can be tested over time. The devices can be tested after storage for one hour to 5 years or, in one implementation, for 10 days to one year. To evaluate the device configuration functional impedance equipment can be used such as a dynamic probing electrochemical device tester.

The testing can be under constant current discharge, constant rate of current change, constant voltage discharge, constant power, constant rate of power change, or any combination. The tests can be combined with mechanical flexion, durability, bending, and compression tests.

Tests can be along the flexing axis of the device test cells or combinations of devices test cells The equipment used for device configuration functional impedance testing can be further configured to measure the tissue electrode functional impedance of the system or can be combined with other equipment for this purpose. Under constant current or constant current change discharge the voltage can be measured. Under constant voltage or constant voltage change discharge the voltage can be measured. The discharge of the device test cells can be based on the target dose. The discharge of the device test cells can be based on the target dose current and time. The discharge of the device test cells can be constant current with a current of 0.01 to 100 mA or, in one implementation, 0.5 mA to 20 mA. The discharge of the device test cells can be considering multiple current levels. The current levels span and exceed the target dose current or target dose current range, The discharge of the device test cells can be constant current at one mA, 2 mA, 3 mA, 4 mA, 5 mA, and 6 mA. Each test can be repeated on the same device test cell or on a newly fabricated device test cell. The discharge can be controlled with a voltage limit. The voltage limit can be 1-100 V or, in one implementation, 5 to 50 V. The voltage limit can be set based on tolerability or other functional concerns. The discharge of the device test cells can be constant voltage at one V, 2 V, 3 V, 4 V, 5 V, and 6 V. The testing can include failure mode cut offs based on a specific parameter. For example, if the initial voltage can be below a threshold the device test cell can be classified a failure mode. A low initial voltage can be below 50 V, below 20 V, below 2 V, below 1.5 V or below 0.5 V. A low initial current can be below 500 uA, below 200 uA, below 50 uA, below 10 uA or below one uA.

The fourth DTIM step includes integrated system modeling. In the fourth DTIM analysis of data from the second or third steps or both steps together. Each of the following analysis can be applied to date from the device test cell discharge or tissue electrode functional impedance of the system or both. Data can be analyzed at current over time, voltage over time, or power over time. Data can be analyzed in isotemporal format. Isotemporal format can be processed to reduce signal to noise including low pass filtering. Isotemporal format can be analyzed from one subject or from multiple subjects or an average across subjects. The tissue electrode functional impedance set of the system can yield an average. Data can be isotemporal format from device test cell discharge or tissue electrode functional impedance of the system are overlaid. There are thus intersection points across these data cells representing the predicted device discharge trajectory. The predicted device discharge trajectory can be based on a scaling factor. The scaling factor can be the number of cells. The predicted device discharge trajectory based on a single test cell can be multiple by the number of cells to provide the predicted device discharge trajectory for that number of cells. The scaling factor can be the area of cells. The area cell scaling factor can be 0.01 to 10000 fold or, in one implementation, 0.1 to 10 fold. The predicted device discharge trajectory based on a single test cell can be multiplied by the relative increase in cell size to provide the predicted device discharge trajectory for that modified cell. Multiple predicted device discharge trajectories are generated in this way. 20-10000 predicted device discharge trajectories can be generated or, in one implementation, 10-100. Each predicted device discharge trajectory associated with specific device configurations can be a design. A successful design yields a predicted device discharge trajectory as set in DTIM step 1. A successful design yields a predicted device discharge trajectory comparable to the target dose. If the target dose can be between 2 mA to 5 mA, over a period of 20 minutes, the successful design will yield a predicted device discharge trajectory of approximately 2 mA to 5 mA, over a period of 20 minutes. A maximum error of less than 100% can be considered successful or, in one implementation, a maximum error of less than 20%. An average error of less than 50% can be considered successful or, in one implementation, a maximum error of less than 20%. A single subject maximum error of less than 100% can be considered successful or, in one implementation, a maximum error of less than 50%. Additional constraints specified in DTIM step 1 can be compared to the predicted device discharge trajectory including maximum voltage, maximum charge, maximum current, maximum power or any combination.

In the fifth DTIM step, the device design can be selected based on the fourth DTIM step and the device can be built. The tests conducted in the third DTIM step are not repeated on the updated device. The performance based on the first DTIM step can be evaluated on the updated device according to the methods of the second DTIM step. The resulting performance can be compared to the predicted device trajectory as in the fourth DTIM steps. This step integrated the architecture layer features described.

The fourth DTIM step can include computational current flow modeling or lumped parameter models of electromagnetic physics simulation. The modeling of the system can include the battery elements, interconnects, separators, heat control elements, electrode interface, electrode-skin-interface, skin, superficial tissue, deep tissue, or any combination of these elements. In one implementation battery elements, interconnect, separators, and electrode interface are modeled as a lumped parameter model. Such models can incorporate the data of the third DTIM step. Each element can be represented as a combination of resistors, capacitors, inductors, batteries, and non-linear elements. In one implementation the electrode, skin, and other tissue elements are modeled in finite element modeling (FEM). Such models can incorporate the data of the second DTIM step. Each tissue can be represented according to its electrical properties including permeability, permittivity, or resistivity. In one implementation the device leverages through-layer thermal modulated electrochemistry to regulate dose. The device enclosure can be selected to have a thermal conductivity of a value greater than 0.002 Wm-1 K-1 or, in one implementation, greater than 0.1 Wm-1 K-1 or, in another implementation, greater than 1.2 Wm-1 K-1. The thickness of the enclosure can be less than one mm or, in one implementation, less than 0.1 mm or, in another implementation, less than 0.01 mm. The distance from the at least one anode or at least one cathode to the inner surface of the enclosure can be less than 2 mm or, in one implementation, less than 0.5 mm or, in another implementation, less than 0.1 mm. In one implementation, there can be no materials between the inner surface of the enclosure and the anode or cathode with thermal conductivity less than 2 Wm-1 K-1 or, in one implementation, not less than 0.2 Wm-1 K-1 or, in another implementation, not less than 0.01 Wm-1 K-1. The thermal properties of the device including the enclosure, the batteries, and related elements are controlled. Upon application of the device to the skin, the temperature of the device increase. The increase reflects several novel factors including the electrochemical activity of the device with a layered architectural design, the heat and gas transfer design of the device, the coupling to the body, or the coupling to the air, or a combination of these factors. The design of the device results in a heating of at least one anode, or at least one cathode, or at least one separator more than 0.01° C. per second or, in one implementation, more than 0.2° C. per second or, in another implementation, more than 0.5° C. per second. In one implementation the enclosure thickness can be 0.15 mm with a thermal conductivity of 0.1 Wm-1 K-1, the distance from the anode to the enclosure can be 0.1 mm. In one implementation the enclosure thickness can be 0.15 mm with a thermal conductivity of 0.2 Wm-1 K-1, the distance from the anode to the enclosure can be 0.05 mm. In one implementation, the enclosure thickness can be 0.5 mm with a thermal conductivity of 0.5 Wm-1 K-1, the distance from the anode to the enclosure can be 0.1 mm. In one implementation, the enclosure can be 0.15 mm thick and the anode heats at a rate of 0.5° C. per second. In one implementation the enclosure can be 0.4 mm thick and the anode heats at a rate of one° C. per second. In one implementation the enclosure can be 0.05 mm thick and the anode heats at a rate of 0.1° C. per second. As the electrochemistry can be temperature dependent, the change in heat changes the device performance. The electrochemistry can be based on the materials and architecture as described. The discharge as can be measured in the fifth DTIM step reflects the integrated design of all elements with through-layer thermal modulated electrochemistry. For a dose of 20 minutes, a design can increase the temperature by 2 degrees within the first 5 minutes. For a dose of 20 minutes with voltage greater than 10 V, a design can increase the temperature by 1° C. within the first 5 minutes. For a dose of 20 minutes with voltage greater than 20 V, a design can increase the temperature by 3 degrees within the first 5 minutes. In one implementation, the anode temperature can increase between one and 2° C. within 5 minutes of application, associated with an increase in current of 2 mA. In one implementation, the anode temperature can increase between 2 and 4° C. within 5 minutes of application, associated with an increase in current of 3 mA. In one implementation, the anode temperature can increase between 2 and 5° C. within 5 minutes of application, associated with an increase in current of 4 mA. In one implementation, the anode temperature can between 4 and 6° C. within 10 minutes of application, associated with a decrease in current of 3 mA. In one implementation, the anode temperature can between 4 and 6° C. within 20 minutes of application, associated with a decrease in current of 4 mA. In one implementation, the anode temperature can between one and 6° C. within 20 minutes of application, associated with a decrease in current of 1.5 mA. In one implementation, the anode temperature can between one and 3° C. within 20 minutes of application, associated with a decrease in current of 2 mA. The device can include a label indicating the temperature for storage, the temperature at use, or the temperature of the tissue, or any combination of these. Through-layer thermal modulated electrochemistry can accelerate discharge dynamics compared to dynamics at room temperature. In one implementation the time to reach the peak target current can be reduced by more than 10% or, in one implementation, less than 20% or, in another implementation, less than 50%. A target dose of 5 mA peak can be reduced by through-layer thermal modulated electrochemistry from one minute to 6 seconds. A target dose of 3 mA peak can be reduced by through-layer thermal modulated electrochemistry from 2 minutes to 45 seconds. A target dose of 20 mA peak can be reduced by through-layer thermal modulated electrochemistry from 5 minutes to one minute. In one implementation the time to reduce the initial voltage can be reduced by more than 15% or, in one implementation, less than 20% or, in another implementation, less than 30%. A voltage reduction from 20 V to 10 V can be reduced by through-layer thermal modulated electrochemistry from 10 minutes to 5 minutes. A voltage reduction from 30 V to 10 V can be reduced by through-layer thermal modulated electrochemistry from 20 minutes to minutes. The device hydrogel can be selected to have a thermal conductivity of a value greater than 0.001 Wm-1 K-1 or, in one implementation, greater than 0.1 Wm-1 K-1 or, in another implementation, greater than 2 Wm-1 K-1. The thickness of the hydrogel can be less than 10 mm but greater than 0 mm or, in one implementation, less than 5 mm or, in another implementation, less than one mm. In one implementation the enclosure can be one mm thick with a thermal conductivity of 0.1 Wm-1 K-1 and the hydrogel can be 5 mm thick with a thermal conductivity of than 0.5 Wm-1 K-1. In one implementation, the enclosure can be 0.5 mm thick with a thermal conductivity of 0.02 Wm-1 K-1 and the hydrogel can be 10 mm thick with a thermal conductivity of than 0.5 Wm-1 K-1. In one implementation the enclosure can be 0.5 mm thick with a thermal conductivity of 0.1 Wm-1 K-1 and the hydrogel can be 10 mm thick with a thermal conductivity of than 0.3 Wm-1 K-1. In one implementation the dose can be 3 mA for 20 minus, the enclosure can be 0.5 mm thick with a thermal conductivity of 0.1 Wm-1 K-1 and the hydrogel can be 15 mm thick. In one implementation, the dose can be 6 mA for 20 minus, the enclosure can be 0.5 mm thick with a thermal conductivity of 0.2 Wm-1 K-1 and the hydrogel can be 10 mm thick. In one implementation the dose can be 3 mA for 20 minus, the enclosure can be 0.5 mm thick with a thermal conductivity of 0.2 Wm-1 K-1 and anode temperature increases by 5° C. in 10 minutes. In one implementation the dose can be 5 mA for 20 minus, the enclosure can be 0.5 mm thick with a thermal conductivity of 0.2 Wm-1 K-1 and anode temperature increases by 3° C. in 10 minutes. In one implementation the dose can be 10 mA for 10 minus, the enclosure can be 0.5 mm thick with a thermal conductivity of 0.2 Wm-1 K-1 and anode temperature increases by 5° C. in 5 minutes as a result of the through-layer thermal modulated electrochemistry. In one implementation the dose can be 10 V for 20 minus, the enclosure can be 0.5 mm thick with a thermal conductivity of 0.2 Wm-1 K-1 and anode temperature increases by 2° C. in 15 minutes. The interconnect serves the dual function of electrical connection and heat sinks.

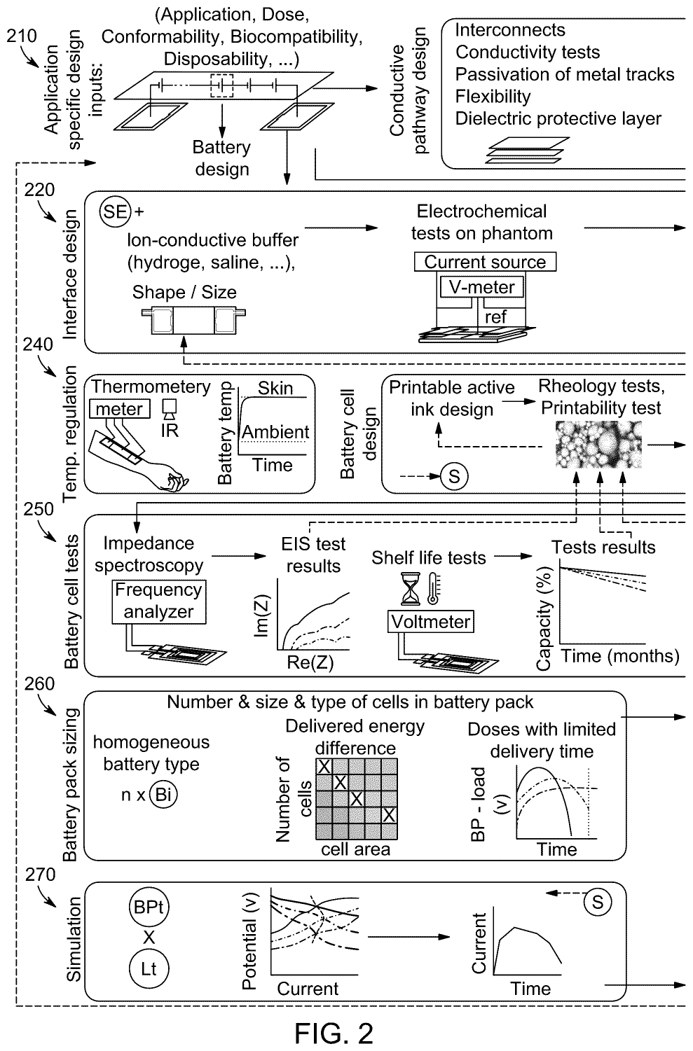

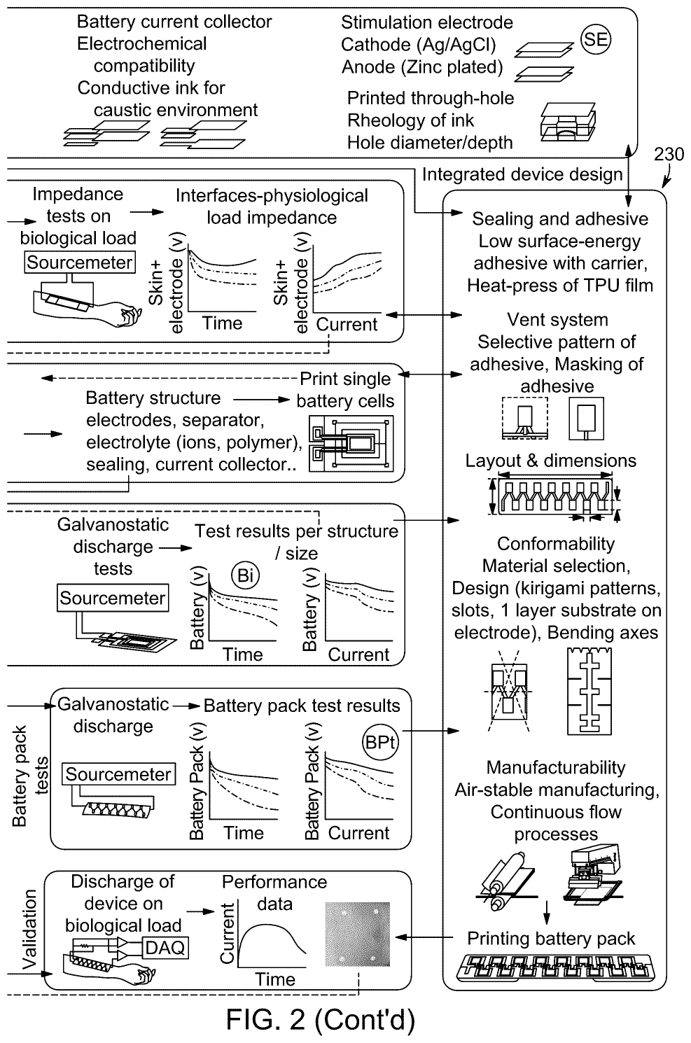

FIG. 2 illustrates a design pipeline enabling wearable disposable electrotherapy, in accordance with some embodiments. A design stage (e.g., stages 210, 220, 230, 240, 250, 260, 270) can incorporate constraints derived from wearable disposable electrotherapy features, design inputs (application-specific dose), and design outputs from other stages to produce design outputs. As the outcomes of each stage impact other stages of the design, device design can be integrated and iterative.

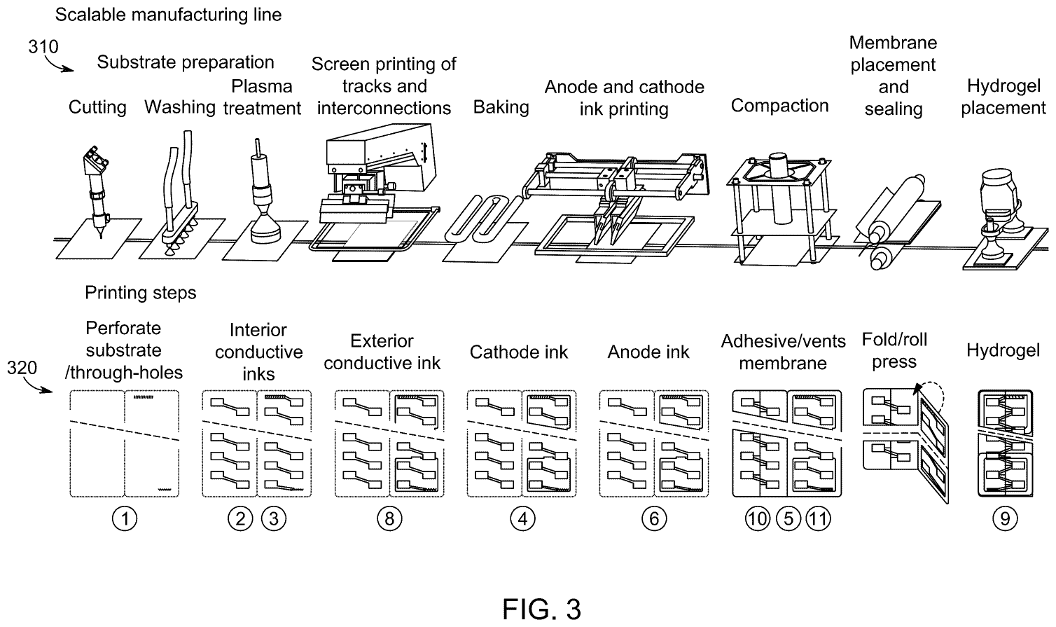

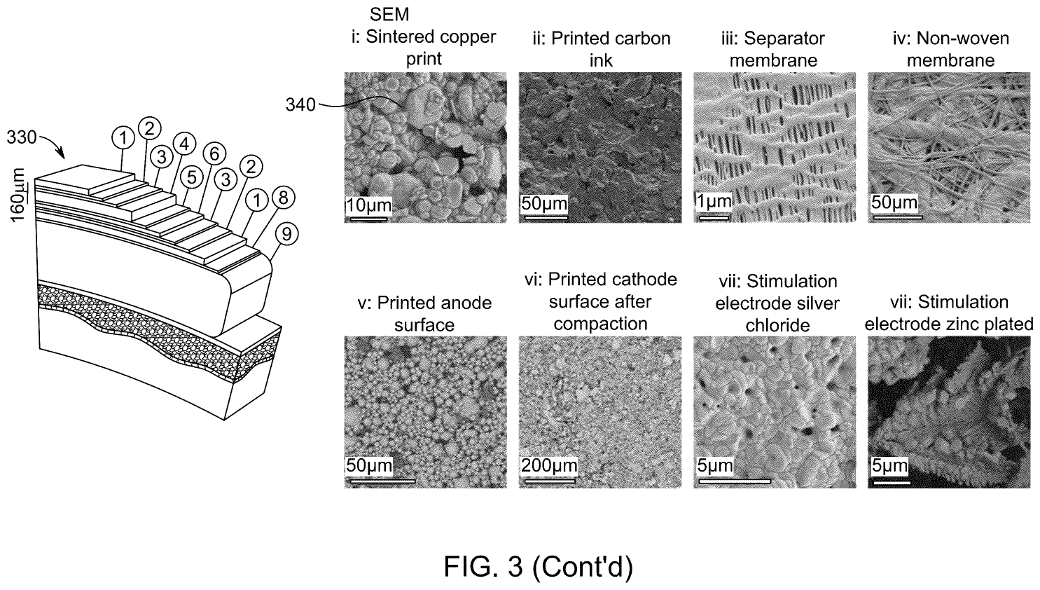

FIG. 3 illustrates wearable disposable electrotherapy manufacturing processes, in accordance with some embodiments. A scalable manufacturing line (image 310) can include substrate preparation, printing conductive/resistive and dielectric inks, curing and baking, printing active materials, compacting, membrane placement and sealing and hydrogel placement. Images 320 illustrate steps of additive manufacturing on a single substrate leading to 3D device architecture. Images 330 illustrate a schematic of layers forming the device including a printed battery pack, interconnects, interface, and enclosure elements. Images 340 illustrate scanning electron microscopy (SEM) of i: sintered printed copper surface, ii: printed conductive carbon ink, iii and iv: surface of porous PP film and non-woven PP film for retaining battery electrolyte as separator or electrolyte bridge, v: printed anode surface, vi: compacted cathode surface vii: corroded surface of printed silver for cathode stimulation electrode, viii: zinc plated surface for anode stimulation electrode. Elements 1-11 labels as per FIG. 1.

### Scalable Manufacture

The following sections describe an example manufacturing process and should not be construed to limit any aspects of this disclosure.

The manufacturing process for disposable wearable electrotherapy devices adapts methods for economic efficiency and high volume (310). These processes are characterized by subtractive and additive approaches to produce an enabling 3D device structure (320, 910a).

Fabrication can begin by heat treating the substrate to ensure its physical stability. This treated substrate can serve as the foundation for the deposition of the components that will comprise the electrotherapeutic device. The substrate can undergo rinsing (e.g., using isopropyl alcohol) to remove contaminants that can affect the adhesion of the components that was printed onto the substrate. Plasma treatment can modify the substrate's surface energy, increasing the polarity of the polymeric substrate, and thus enhancing the ink adhesion and ensuring effective sealing in the downstream processes.

The conductive pathways required by device design can be formed by printing conductive and dielectric inks providing required functionalities. Copper ink, conductive carbon ink, zinc ink, Ag/AgCl ink and dielectric ink can be among the materials used to construct the various elements of the device. Each fabrication stage can be followed by drying and baking steps to fix the features. After forming these features, the battery anode and cathode materials can be deposited on the battery current collectors. Electroplating (for microgram accuracy in the deposition of active materials), screen printing (for thinner layers), and/or stencil printing (for controlled deposition thickness, favorable for cathode deposition) can be applied for additive manufacture of device layers. The selected inks formulation supports air-stable production, with ink rheology optimized for the printing method while achieving the electrical performance required such as discharge rate. A compaction process can increase electrode density and lead to decreased internal battery resistance for the cathode material.

The device can have a parallel plate battery structure where a separator membrane is assembled between the battery electrodes to electrically isolate them while allowing ionic transport. The sealing stage closes the battery pack between the two enclosure sheets with double-sided tape with an adhesive tailored for low surface energy polymer surfaces. During the sealing stage, the venting system is created by selective masking of adhesive (1020, 1040, and 1130, 1160). After sealing, electrolyte can be introduced to cells resulting in an active, charged battery unit. In a final fabrication step, hydrogel or non-woven felt (later soaked with electrolyte) as stimulation ion-conductive buffer is applied to the outer surface. 330 shows a resulting stack of different layers forming the device.

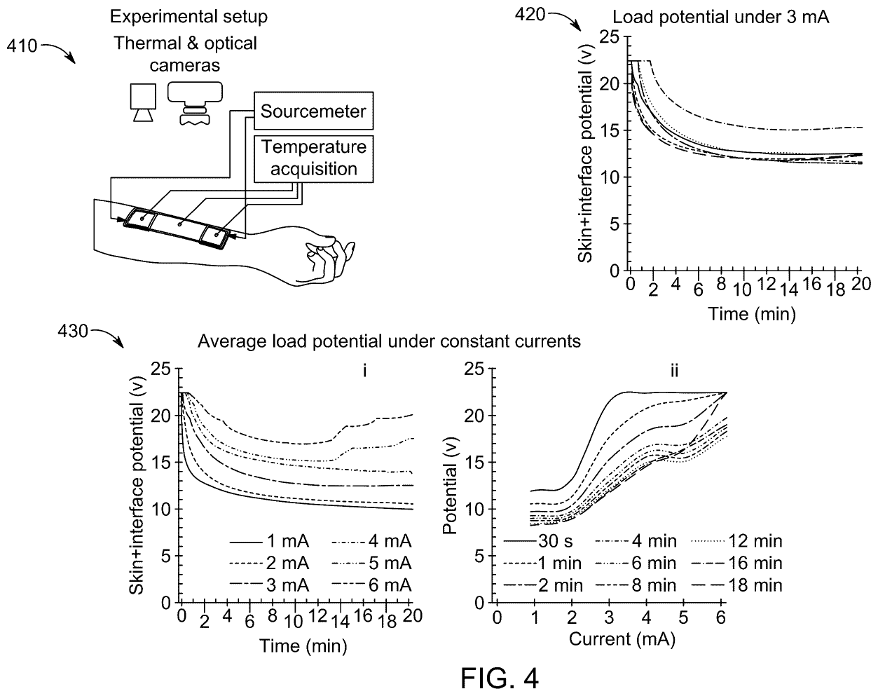

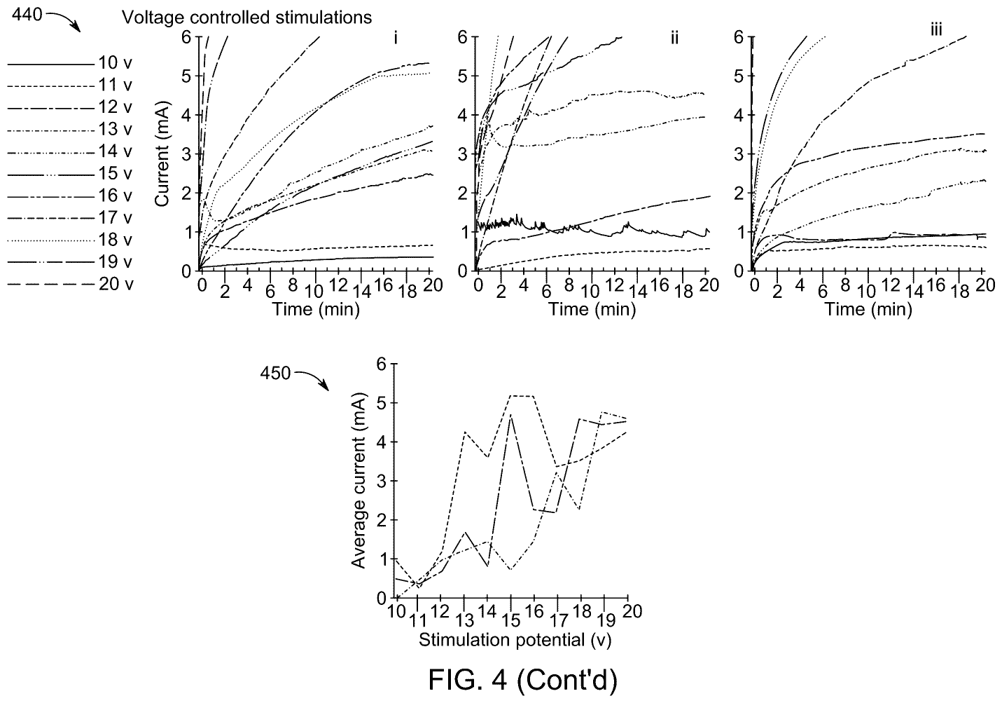

FIG. 4 illustrates a load characterization for aa wearable disposable electrotherapy device, in accordance with some embodiments. 410 shows an experimental setup using the interface test device. 420 shows a potential of load (e.g., user skin and electrical interface) under 3 mA over 20 min for individual subjects (colored lines) and average (black line). Plot 430 shows an average load potential over 20 minutes under constant currents (used to size battery packs), associated ii: isotemporal V-I curves (used to simulate battery pack discharge). Plot 440 shows voltage-controlled stimulations over 20 minutes for three subjects (i, ii, iii). Plot 450 shows average current (over the active duration) for fixed applied voltages.

Device design incudes interface-physiological load characterization. Unlike that of stimulation devices that are current-controlled or voltage-controlled, the output of the disposable wearable electrotherapy device is governed by the electrical coupling of the dynamic behavior of the battery pack, the interfaces (e.g., the electrode and the ion-conductive buffer), and the physiological load characteristics. For each application (target electrotherapy dose) the interfaces-physiological impedance can be characterized as part of device design (FIG. 2). For this, a device with only the interface components (e.g., devoid of battery pack material) is powered by a sourcemeter (410). The sourcemeter is connected to the stimulating electrodes and programmed to contrast currents (around the target electrotherapy dose) with a voltage-compliance (reflecting the maximum expected from a given battery pack). Because the device applies electrotherapy to the body in a pre-energized state, for interfaces-physiological impedance testing the source-meter can be activated (to the compliance voltage) prior to placement of the interface components on the body.

To characterize the interfaces-physiological impedance load for the exemplary device, interface-components were applied to subjects' forearms connected to sourcemeters providing 1-6 mA current-controlled with a 22.4 V compliance. Other experiments considered the response to constant voltage stimulation with 10-20 V in 1 V increments (17 total conditions across n=10 subjects).

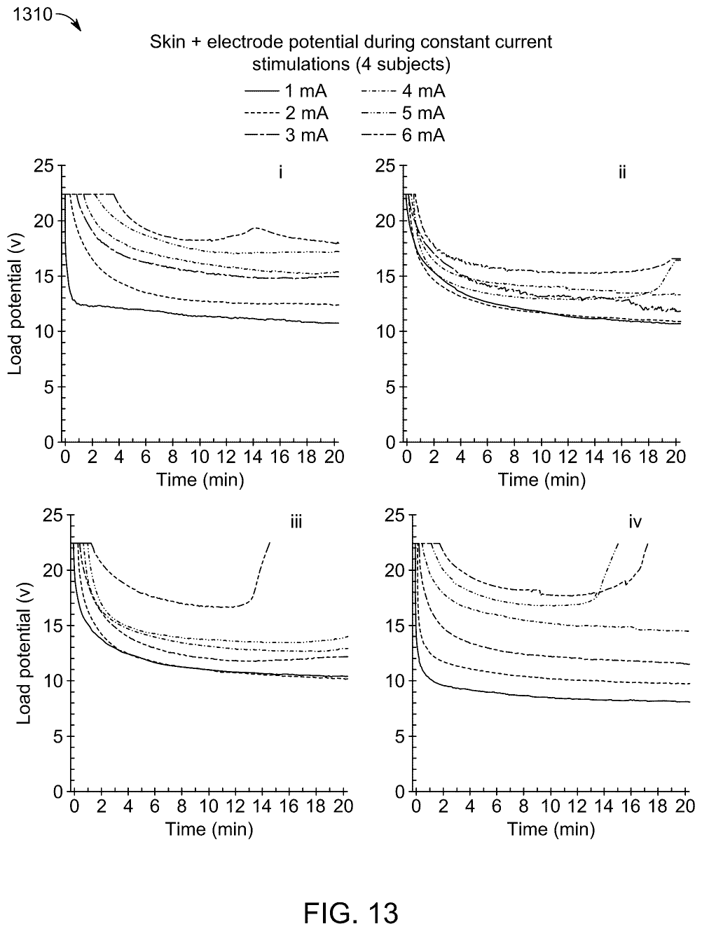

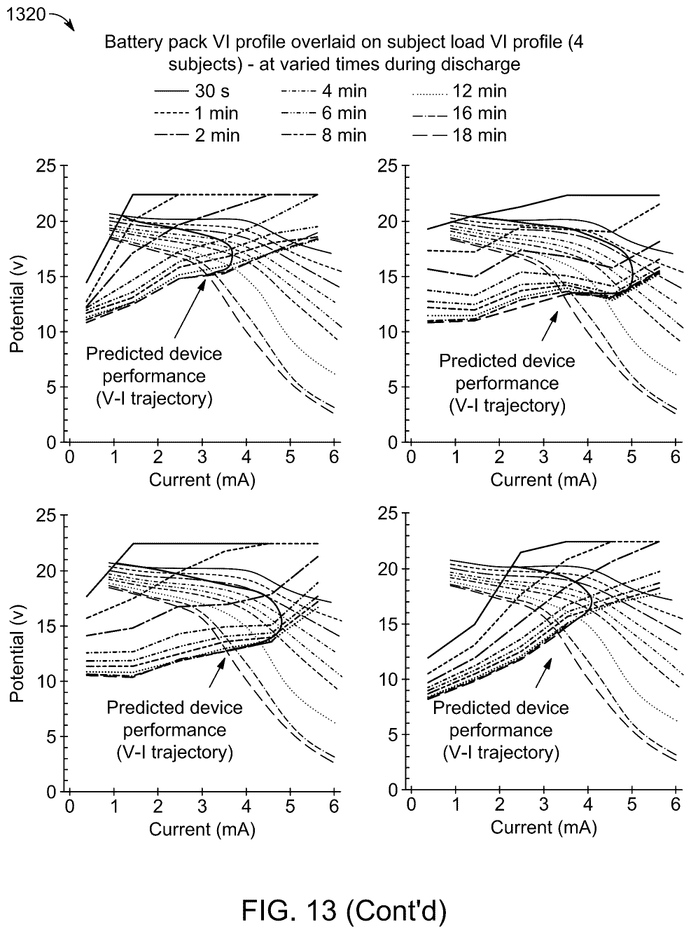

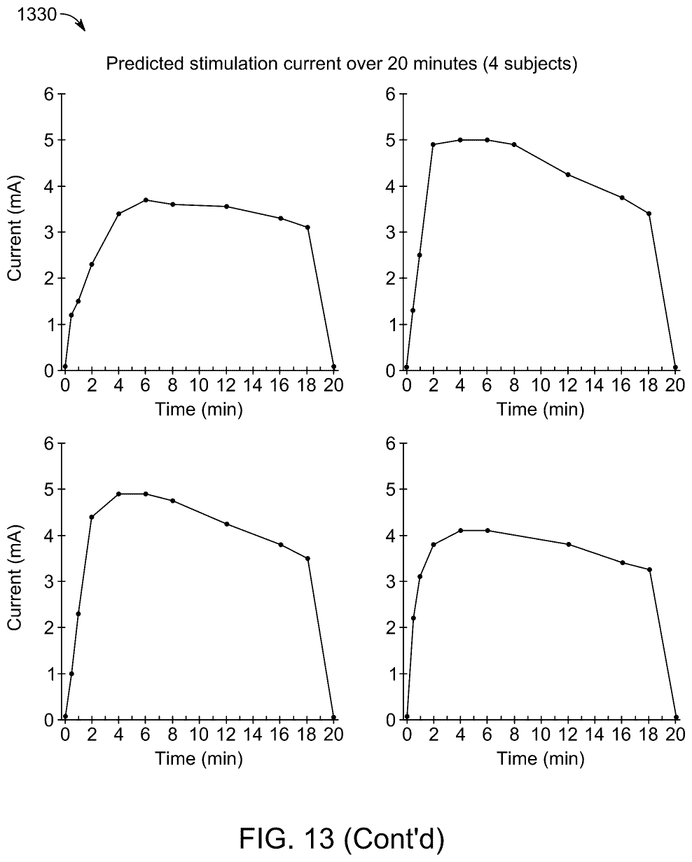

Under constant 3 mA stimulation, voltage gradually decreased in each subject (420). On average, across subjects, voltage decreased gradually under 1-4 mA constant currents but increases at ˜14 min for 5-6 mA current (440i); these relationships are summarized in isotemporal lines (average load, FIG. 440ii; individual subjects' load, FIG. 13) for subsequent battery pack design. The interfaces-physiological impedance is dynamic as a function of applied current and time, reflecting nonlinear processes at the interface electrode and skin.

Batteries do not provide constant current and their internal voltage/impedance is a nonlinear function of the current drawn; this creates a complex interdependence between source and interfaces-physiological impedance load. Subsequent design steps (FIG. 2) show how interfaces-physiological impedance data informs novel battery pack design that considers (and indeed leverages as a self-regulating mechanism of battery voltage) a dynamic coupling between batteries and interfaces-physiological impedance load.

Constant voltage stimulation (10-20 V) can produce unreliable current (440). Current fluctuates on an experiment-wise basis over time. Current may not be monotonic with time or applied voltage. These results show that voltage-controlled stimulation (i.e. from an idealized battery) may not be reliable for stimulation across interface and physiological impedance loads. The battery packs for the wearable disposable electrotherapy device can be configured to change in voltage with interfaces-physiological impedance load.

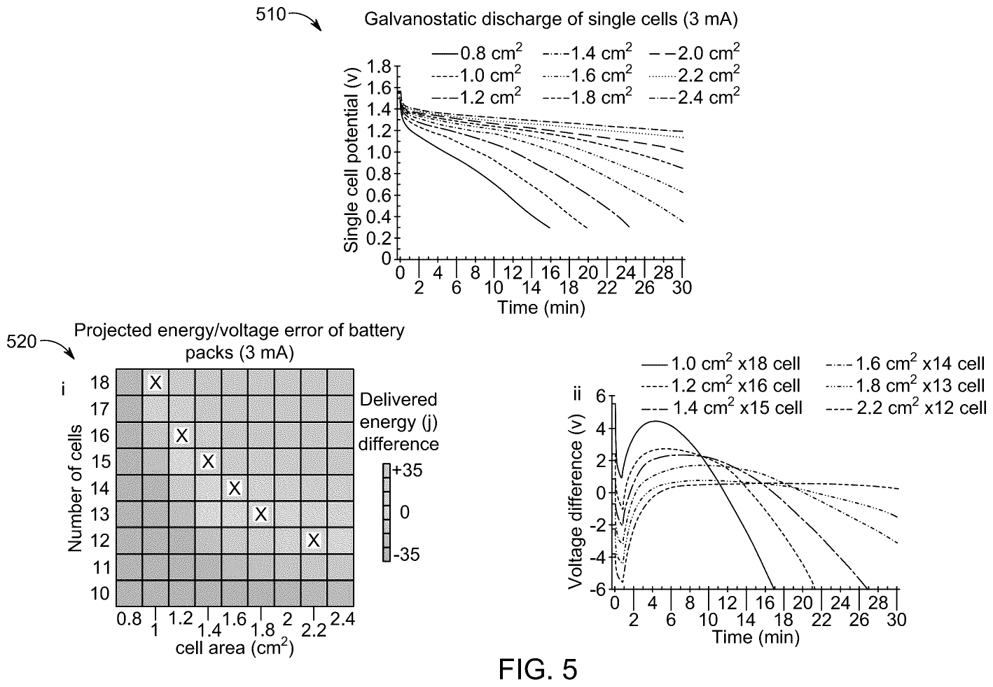

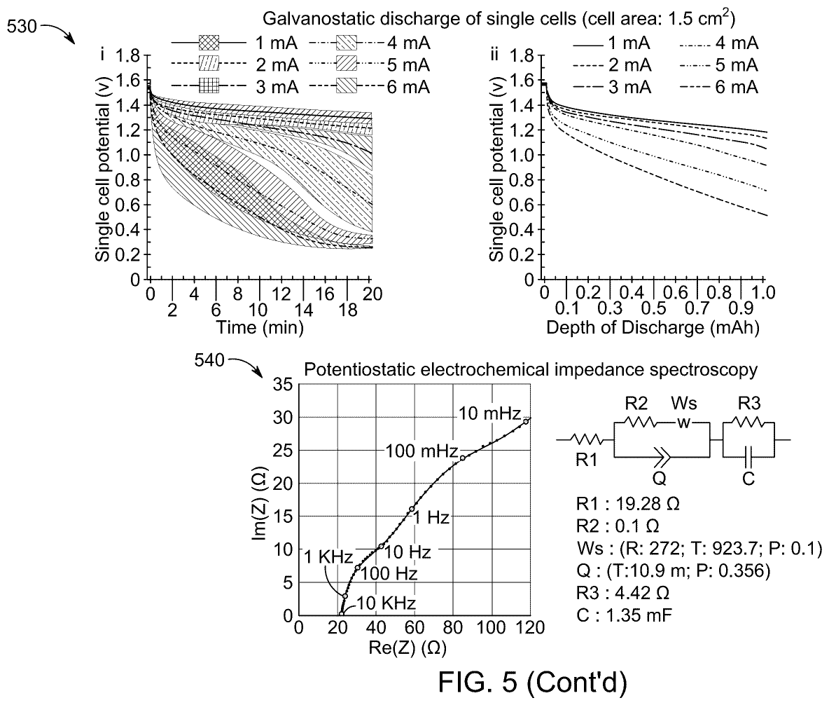

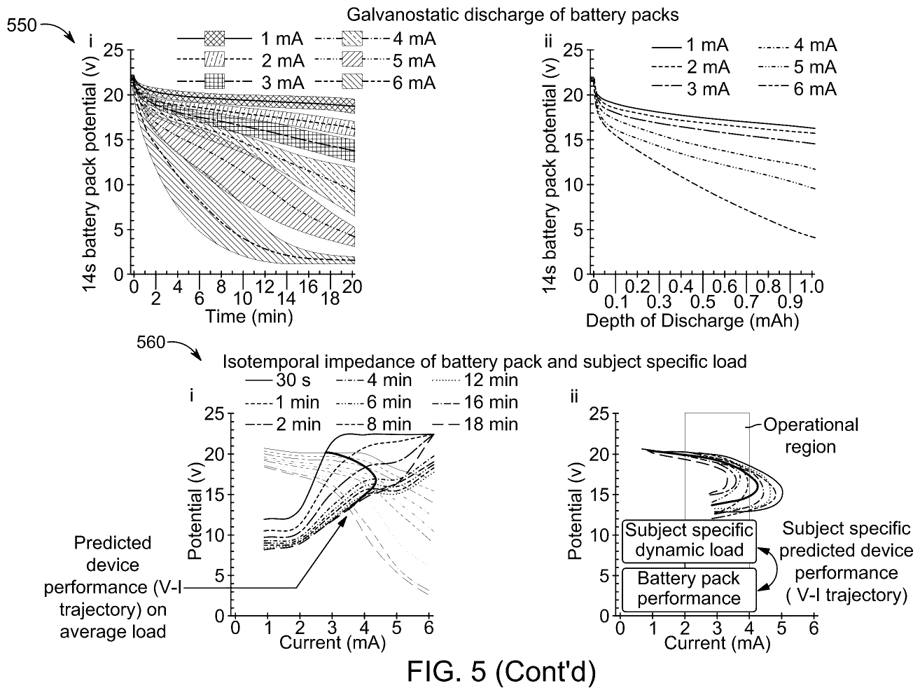

FIG. 5 illustrates battery cell and pack design (sizing), validation, and stimulation for a wearable disposable electrotherapy device, in accordance with some embodiments. Plot 510 shows a galvanostatic discharge curve of single-cell batteries (average) with different cell sizes under 3 mA current over 30 min; Plot 520 shows i). projected energy (j) error of battery packs with 10 to 18 cells which use cells with different sizes under 3 mA discharge against average of energy required to stimulate load with 3 mA during 20 min (x: selected designs for next stage); 520 ii) voltage differences between six battery pack designs (selected based on minimal projected energy error) and voltage of load over 30 minutes at 3 mA. Plot 530 shows a galvanostatic discharge curve of single cells with sized area of 1.5 cm². Plot 540 shows a potentiostatic electrochemical impedance spectroscopy of single cell per one cm² area over frequency range of 10 mHz to 10 kHz with bias equal to open-circuit voltage (OCV) of freshly assembled battery and fitted model; Plot 550 shows a galvanostatic discharge curve of sized (14 cell, 1.5 cm² area) battery pack, i: over 20 min time, ii: over-discharge depth. Plot 560 shows i) an isotemporal V-I curve of sized battery pack discharge (dashed lines) with overlaid average isotemporal V-I load curve (solid line). The intersection of these lines is the predicted discharge for the sized device into the average load (black line) (Plot 560 ii) Given each subject's V-I load curve, a subject-specific (colored lines) and average (black line) sized device discharge is simulated.

The device includes steps for battery pack design. For the exemplary specific target dose (3±1 mA, 20 minutes) and associated interfaces-physiological load dynamics, a homogeneous battery pack (power source with limited voltage and current) is designed. For a set of single battery cell electrochemistry parameters (ink formulation, screen thickness, separator type, electrolyte composition) and device structure (3D connectivity, fabrication/sealing technique) configured in prior stages. Battery peak performance can be improved by adjusting battery cell size and the number of cells in the battery pack.

An experiment conducted an array of single battery cell discharge tests, maintaining a consistent discharge rate of 3 mA over 30 minutes (510). During these tests, cell sizes ranged from 0.8 cm² to 2.4 cm², with constant areal densities of the active materials. The experiment scaled these recorded voltages by factors ranging from 10 to 18 to predict associated projected battery pack voltages under a 3 mA discharge rate. These values were compared with potentials across the interfaces-physiological load under 3 mA for 20 minute stimulation (FIG. 4). The objective was to identify a battery size and the required number of cells that minimized the voltage variation throughout the 20 minute span (520i) while also ensuring the battery pack neither depleted prematurely nor persisted substantially beyond the 20 minutes (520ii, 1250). At this stage, the battery pack sizing (optimized by cell types, sizes and number) was based on minimizing the energy difference, over the targeted dose, between the battery pack and load by:

$$min \{ {\int }_{t = 0}^{{T}_{dose}} ( {V}_{B , N , type} ( Idose , t ) - {V}_{L} ( Idose , t ) ) dt \} \qquad ( Eq . 2 )$$

with respect to battery size and time for each of N possible battery cell choices, and

$${V}_{B} ( {I}_{dose} , t ) = {\sum }_{i = 1}^{N} {V}_{C} ( {I}_{dose} , t , type ( i ) , size ( i ) ) \qquad ( 3 )$$

where V_{C} (Idose, t, type (i), size (i)) is the voltage of a single battery cell at time t for a given current-controlled discharge (I_{dose}) applied over time (T_{dose}), which are set to the target dose. The battery type and size of each battery cell affects its voltage, and hence affects the cumulative battery pack voltage. For the exemplary device, the selection of battery chemistry and a homogenous pack design reduces the optimization to cell area and number. Following this analysis, a configuration of 1.5 cm² with 14 cells per pack was deemed optimal for the specified target dose.

The experiment then measured (n=72) the average potential of single 1.5 cm² cells over a 20 minute span across various discharge rates (1-6 mA); both average performance (530i, solid line) and performance variation due to variation in battery fabrication (530i, shaded). The average potential of the cells for up to a discharge depth of one mAh was calculated (530ii).

The experiment further characterized the performance of single battery cells using potentiostatic electrochemical impedance spectroscopy (EIS) and developed an associated circuit analog model (540). The lumped-parameter device impedance model reflects electrochemical processes occurring in the cell. These single cell data are then used to inform iterations of battery chemistry (1280, 1290, 1295) and battery pack design.

Device design incudes steps of verification and system stimulation. Having designed the size and quantity of batteries for each pack for the exemplary target dose and load, experimental trials fabricated and subjected these battery packs to galvanostatic discharge tests (n=60 battery packs). The average potential of battery packs over a 20 minute span across various discharge rates (1-6 mA) was measured; both average performance (550i, solid line) and performance variation due to variation in battery fabrication (550i, shaded) was measured. The average potential of the battery packs for up to a discharge depth of one mAh was calculated (550ii).

The battery pack discharge dynamics are represented in isotemporal lines (dashed lines; 560i). These are overlaid with isotemporal lines from the load average (solid line; 560i). For each time point, the interaction of these lines is represented (solid line; 560i). The temporal evolution (over the stimulation duration) of the intersections between the voltage-current characteristics of the battery and of the load is the projected device output. This corresponds to the simulated disposable wearable electrotherapy device dosing for an average associated interfaces-physiological impedance load. The projected output is then analyzed across multiple subjects, based on their respective load analysis (FIG. 13; 560ii). These trajectories show the projected operational envelope of the device (within a safe range). Reflecting the device design (sizing and battery cell chemistry; 1270), trajectories show the current will initially ramp up with limited decrease in voltage. As stimulation proceeds, the current can reach a maximum value, after which the voltage decreases substantially, limiting current delivery. A further decrease in voltage can ramp the current down.

As the device is thin and battery pack (polymer) elements can be impacted by temperature, the experiments quantified device operational temperature. Sensors embedded in the device (on the anode stimulation electrode, device center, and cathode stimulation electrode) showed devices quickly (τ=40 s) warm from room temperature to surface skin temperature (1410), with an incrementally higher temperature at the cathode (1430, consistent with enhanced erythema). Associated warming at the skin surface was confirmed by thermal imaging 1430. The experiments enabled development of a computational model of device heat transfer (1420), consistent with experimental findings. These results confirm that primary temperature increase reflects device warming to body surface temperature, consistent with conventional electrotherapies, during the initial (ramp up) discharge period.

Stability (shelf-life) testing of the exemplary Disposable Wearable Electrotherapy pack assessed the battery pack's capacity. Stability is controlled according to device design, In one device, over 15 days, the average voltage decreased by 5.25 mV per cell daily. Allowing for a ˜20% decrease (e.g., from 1.55 V to 1.2 V per cell), the results indicate a stability of 66.7 days (i.e., over two months of stability at the prototype stage). In one device, over 15 days, the average voltage decreased by 1 mV per cell daily. In one device, over 15 days, the average voltage decreased by 0.1 mV per cell daily. In one device, over 15 days, the average voltage decreased by 9 mV per cell daily. Device stability is controlled through device design according to the application of use. In an implementation, battery life can be increased by methods of replenishing battery capacity as known in the art such as recharging or adding active material.

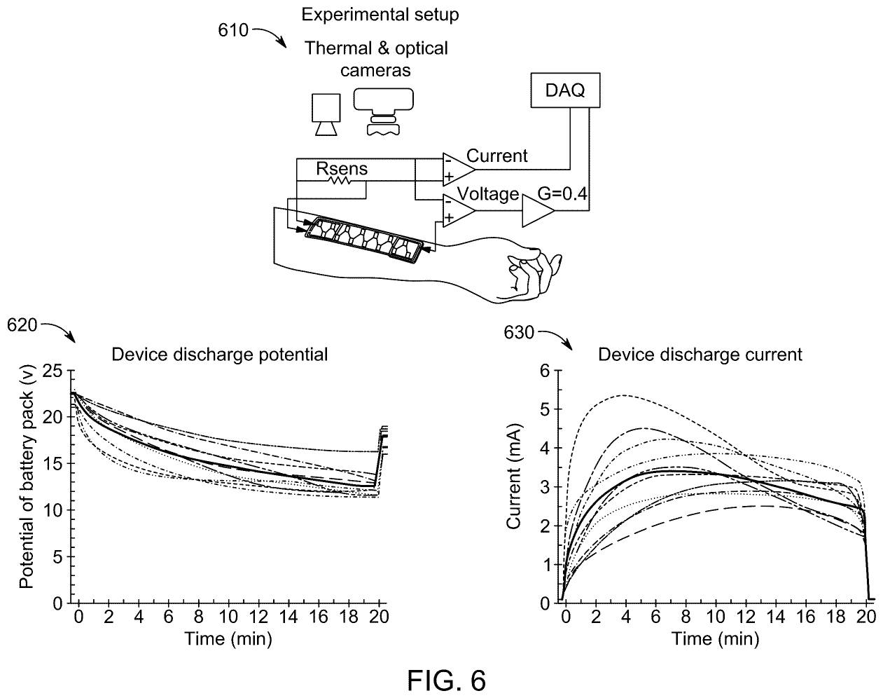

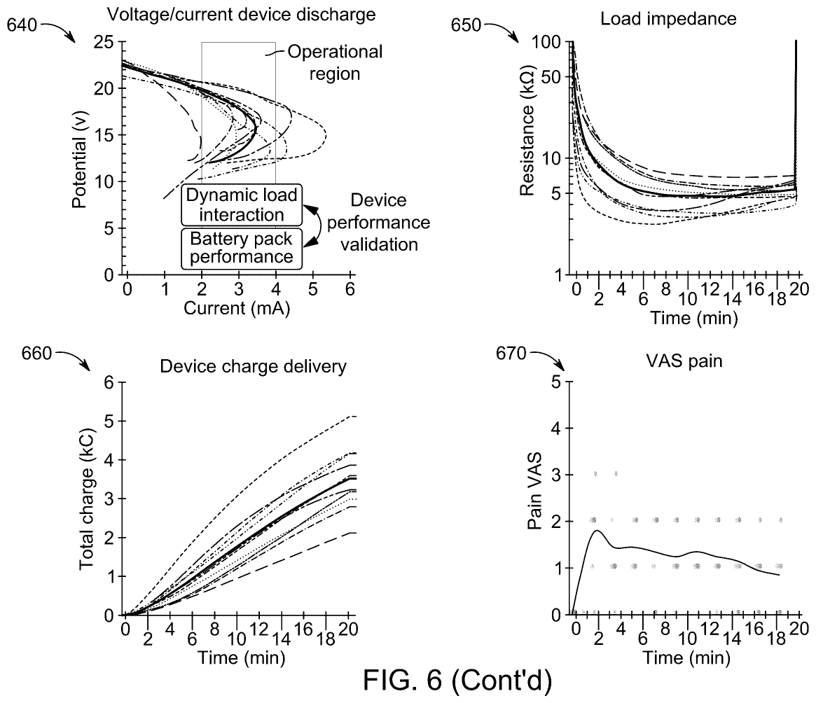

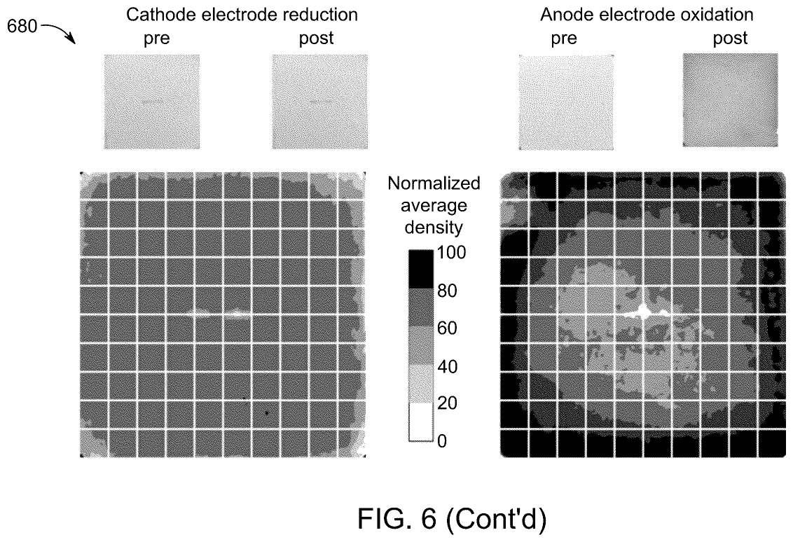

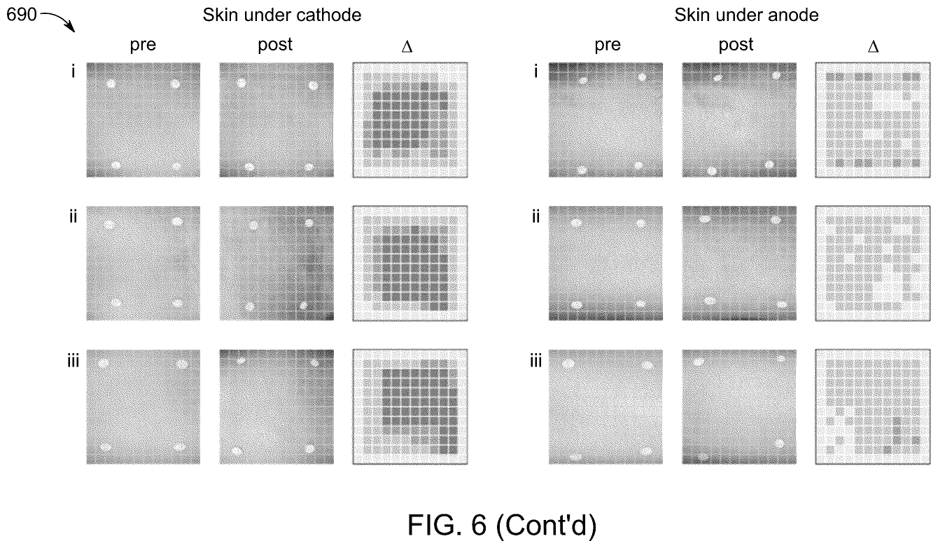

FIG. 6 illustrates validation of exemplary wearable disposable electrotherapy self-limited discharge, in accordance with some embodiments. Plot 610 shows experimental setup for the target application across subjects' forearms. Individual subjects (colored lines) and average (black) lines are shown. Plot 620 shows voltage of battery packs and Plot 630 shows output current over 20 minutes of stimulation. Plot 640 shows a V-I curve of device throughout the stimulation. The experiments compared these measured discharges with device-design simulation (FIG. 560ii). Plot 650 shows impedance of the load (electrodes+skin), 660 shows cumulative charge delivered across the load, and plot 670 shows pain levels over the 20 minutes of stimulation. Plot 680 shows a distribution of stimulation cathode electrode reduction and anode electrode oxidation. Plot 690 shows skin redness relative heat map under stimulation cathode electrode and under anode electrode, for three subjects (i, ii, iii).

With chemistry and sizing designed for the exemplary application, the exemplary disposable wearable electrotherapy device discharge performance was validated (n=10 subjects; 610). Devices satisfied all other requirements, with a final thickness of 1.25±0.07 mm and weight of 10.5±0.1 g. Devices were applied to the skin (t=0) for 20 min, and the generated voltage (620) and current (630) measured. Across subjects voltage, gradually decreased while current gradually increased (τ=8.7±3.4 min) to a peak (3.6±0.8 mA) while current was sustained. A further decrease in voltage reduces current. Stimulation was sustained for the 20-minute application (average voltage 15.4±2.4 V; average current 2.8±0.7 mA; average power 41.8±12 mW). As the device was removed, the current was progressively aborted (τ=5.0±0.5 s).

Voltage-current trajectories (640) exemplify dose control for the disposable wearable electrotherapy device and performance along system-design simulations (compare with 560ii). Discharge is neither strictly current-controlled nor voltage-controlled, but results for each subject were governed by battery pack design given the dynamic impedance response. Load impedance initially decreased with current application (650) but reliably plateaued and normalized across subjects (range 4-7 kΩ at 18 min).

Discomfort during electrotherapy was assessed by conventional VAS rating at 11 time points during stimulation. Stimulation was well tolerated (average pain VAS 1.1±0.75; across 110 measurements never >3; 670), with expected transient erythema and no lasting skin irritation. Subject's transient and mild perceptions of current flow (e.g. “tingling”) are consistent with functional electrical delivery (e.g., activation of nerves).

Uniformity of current delivery was assessed across electrodes and skin surfaces. Charge uniformity was imaged (pre/post discharge, 2D optical scan) at the anode stimulation electrode, as evidenced by oxidation, and at the cathode electrode, as evidenced by gas evolution (680). Average normalized charge density was uniform at the cathode and moderately higher at electrode edges (annular) at the anode. Stimulation uniformity at the skin was assessed by high-resolution photography (pre/post discharge) of skin erythema (690). Immediately post-discharge, erythema was relatively uniform under the cathode while milder and punctate under the anode. At appropriate doses, erythema is expected and transient, non-hazardous and consistent with skin stimulation, and, in the context of iontophoretic drug delivery and wound healing, desired.

Functional conformability is validated through the combination of impedance stability (650), current density uniformity (680,i), and tolerability (670). By achieving an electronics-absent design with thickness of battery pack (all layers) ranging 400-700 μm thickness (1020), flexibility is governed by the design or layout of the battery pack. Given one embodiment of PET flexural modulus (1.30-4.69 gigapascals (GPa)), sheet thickness (100 μm), and device to skin surface ratio <0.3, a >97% comfortability is expected. Given one embodiment of enclosure flexural modulus (0.1-6 gigapascals (GPa)) and appropriate sheet thickness is selected (200 μm). Given one embodiment of enclosure flexural modulus (20-60 gigapascals (GPa)) and appropriate sheet thickness is selected (40 μm). Given one embodiment of enclosure flexural modulus (0.01-0.2 gigapascals (GPa)) and appropriate sheet thickness is selected (50 μm). The selection of stiffness and thickness is based on selected materials and device application. The selection of stiffness can be governed by through-holes design as explained.

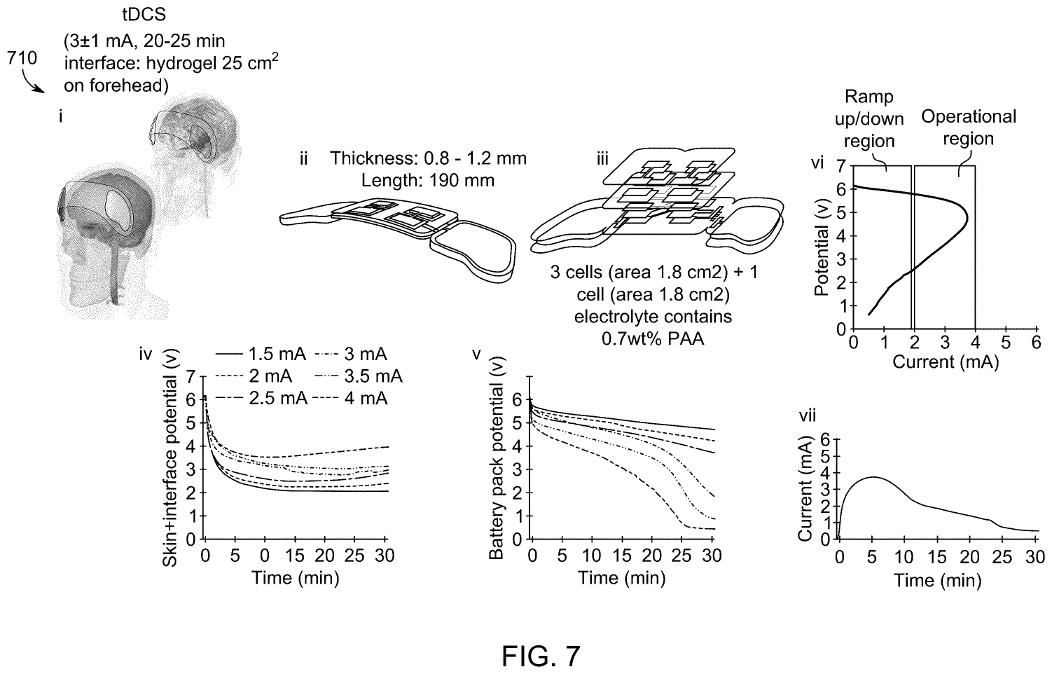

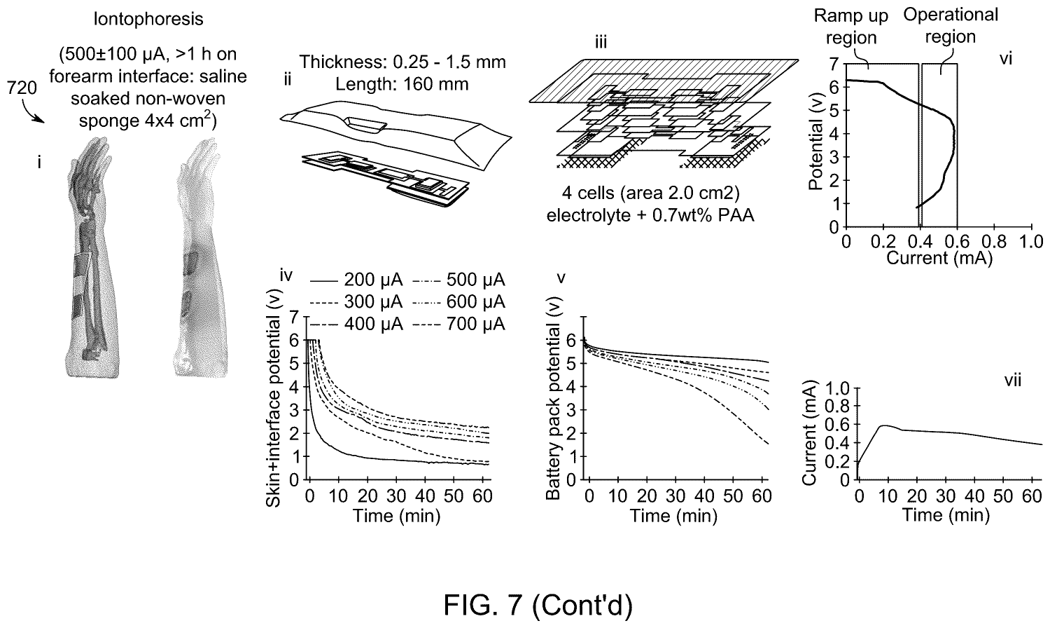

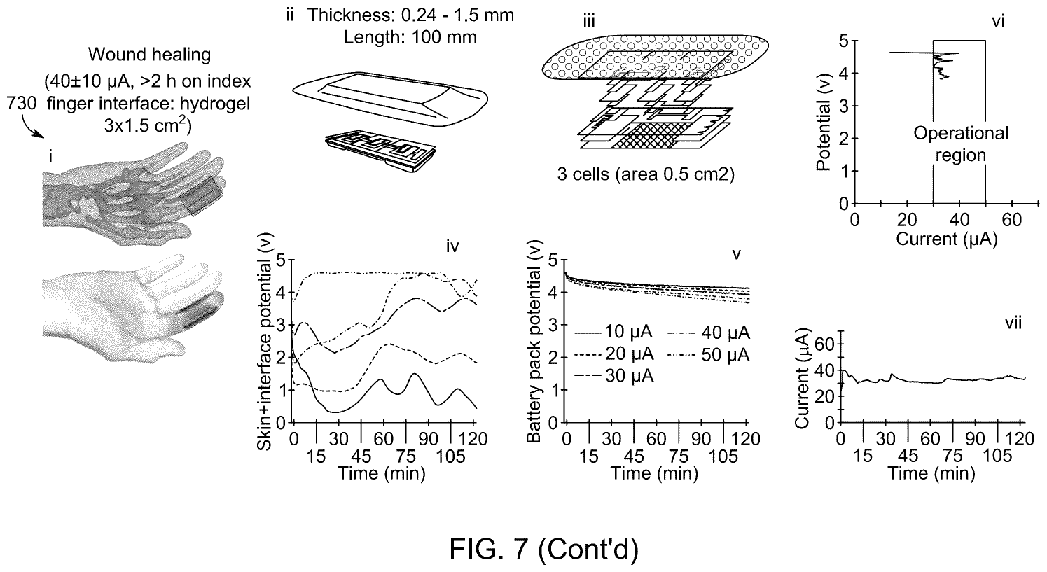

FIG. 7 illustrates an application specific wearable disposable electrotherapy design, in accordance with some embodiments. The design pipeline validated for the exemplary device is applied to three use-cases: tDCS (710), iontophoresis (720) and accelerated wound healing (730) selected for efficacy supported by dozens of RCTs such that a wearable disposable electrotherapy needs to reproduce the dose (design input). i) For each application, high-resolution magnetic resonance imaging (MRI)-derived models simulate the desired tissue current delivery. ii) The outcomes of an integrated design process are application specific Wearable Disposable Electrotherapy devices, thicknesses scaled by ten times for clarity. iii) Exploded view of layered geometry of active materials and interfaces. iv) These designs are the outcome of a process including application dose-specific interfaces-physiological load impedance measurements, informing battery cell design and battery pack sizing. v) Galvanostatic battery pack prototype discharge informs vi) isotemporal device simulation. vii) Application of assembled devices to subjects produces successful outputs.

This section demonstrates three applications of wearable disposable electrotherapy using the directed design framework (FIG. 2). For each application, the electrotherapy dose and mechanical consideration of application serves as the design input to the wearable disposable electrotherapy analog and the validation of design outputs. The range of performance (design inputs) of these applications and exemplary device are selected to demonstrate the platform's flexibility to broad electrotherapy applications. Current spans at least two orders of magnitude (30 uA to 3 mA), duration (˜20 min to >2 hours) with 0.5-2 cm² battery packs of 4-14 cells. Both homogeneous and inhomogeneous battery pack sizing are demonstrated. Application-specific interfaces span at least 4.5 to 25 cm² contacts, hydrogel or nonwoven sponge ion conductors, and varied stimulation anode/cathode materials (copper, zinc, silver, carbon and silver chloride). Battery packs and interface are packaged into application specific enclosures (18 to 132 cm²), with mechanical design supporting comfortability. Variations in interfaces and target doses result in different interfaces-physical impedance loads, reflecting nonlinear dynamics of the load, ‘which are accounted for isotemporal-trajectory theory.

Transcranial Direct Current Stimulation (tDCS) is a non-invasive brain stimulation technique, trialed for a range of neurological and psychiatric disorders. A typical dose is ‘bi-frontal’ 1-4 mA (with 30 second ramp up/down), 20-30 min, ˜25 cm² electrode on the EEG 10-10 F3/F4 scalp positions. A bi-frontal wearable disposable electrotherapy device design input was configured with electrodes below the hairline, using 3±one mA DC average to provide a target electric field to the frontal cortex (max 2.4 V/m at the dorsolateral prefrontal cortex; 710) with ramp up τ>30 s and instantaneous peak current <4 mA. This design was based on interfaces-physiological impedance load under constant currents (1.5-4 mA; FIG. 710iv). An integrated system design (FIG. 2) produced an articulated device architecture (710i) including four inhomogenous cells (710ii). Conformability is enhanced using a single substrate over the interface (920biv) and bend-line cuts in the substrate, which facilitate deformation to the forehead (920bii). To deliver the prescribed dose, battery pack sizing (eq. 2) resulted in an inhomogeneous configuration with 3 cells (1.8 cm²) with 34 wt % KOH electrolyte and one cell (1.8 cm²) with 0.7 wt % PAA polymer added to electrolyte that limits the peak current (1270). Designed devices were prototyped according to the manufacturing process (FIG. 3) and validated on human subjects (710vii) demonstrating discharge performance within design specification and matching design theory.

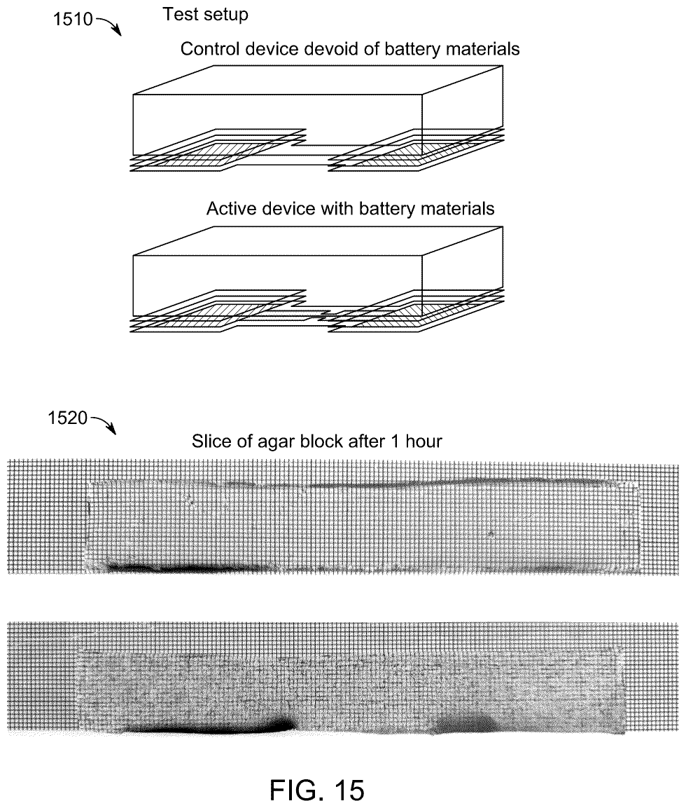

Iontophoresis is an established therapy passing DC through the skin for applications including hyperhidrosis and transdermal drug delivery. A conventional iontophoretic dose uses ion-carrier non-woven sponge interface (4×4 cm) with charge rated dose >30 mA-min (˜1.8 C); when applied for 60 min sustaining an average current of about 500 uA (34). These served as the design inputs for the iontophoresis application of the wearable disposable electrotherapy device (720). System design (FIG. 2) yielded a wearable disposable electrotherapy iontophoresis design (720ii) using four homogeneous cells (2.0 cm²) with 0.7 wt % PAA polymer-supplemented electrolyte to self-limit the peak current. The conformability was enhanced by narrower substrate between two interfaces (920iii). Current flow simulation predicts resulting charge density of 31.2 uA/cm² per second at the device-skin interface (720i). According to isotemporal trajectory theory, the design was developed by measuring the interfaces-physiological impedance load under constant currents (200-700 uA; 720iv). The battery pack was accordingly sized and discharged (720v) and the iontophoresis-application wearable disposable electrotherapy device built (FIG. 3). Using a skin phantom, enhanced diffusion of ionic dye (molecular weight like the range of common drugs used in iontophoresis) was verified (FIG. 15). Finally, devices were validated in human trials to produce the prescribed discharge performance (average current 472 μA, 32.7 mA-min in 60 min; 720vii)

Electrical stimulation can accelerate wound healing. Effective doses include low-intensity DC at 30-50 μA average over >2 hours. An integrated design process (FIG. 2) resulted in a wound healing-application wearable disposable electrotherapy design including three battery cells, and stimulation electrode hydrogels on both sides of a wound dressing pad. The manufactured device is then placed on a skin adhesive for bandages. The substrate is made from a stretchable thermoplastic film with slots between each battery cell for additional flexibility (730ii, 920ii). During heat press sealing, the stretchable thermoplastic film undergoes copolymerization, eliminating the need for a separate adhesive layer between the two substrates. Using an interface device and sourcemeter (10-50 uA), the interfaces-physiological impedance load was determined. Battery packs were sized according to isotemporal trajectory theory and the device was built accordingly. Current flow simulation predicted a resulting largely uniform current density through the targeted region (730ii). Wearable disposable electrotherapy wound healing devices were then validated (730vii) demonstrating discharge performance within design inputs and matching design theory.

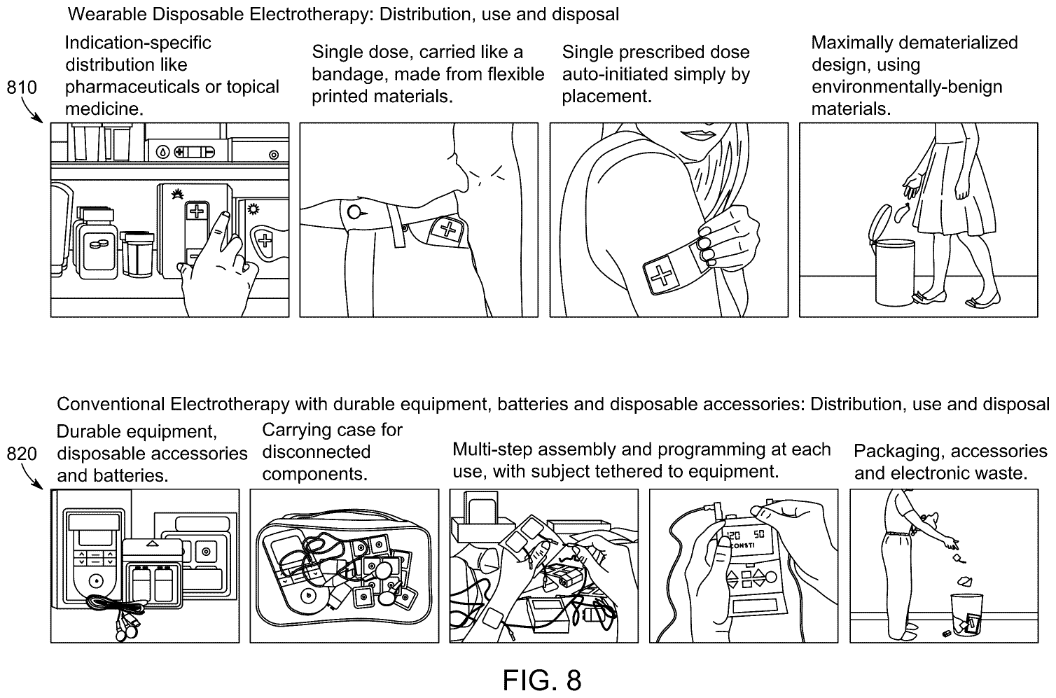

FIG. 8 illustrates several uses for the wearable disposable electrotherapy device. The wearable disposable electrotherapy device eliminates barriers to use of traditional electrotherapy, and facilitates distribution akin to pharmacotherapy or topical medicine (images 810, left to right). Distribution: The wearable disposable electrotherapy device can be dispensed in application-specific single-dose strips, like the distribution of drugs or topical medicine. In contrast, conventional electrotherapy devices have both durable and disposable components and require dedicated power sources. Carrying: Each wearable disposable electrotherapy, containing a single dose, is used like a bandage, while transporting conventional electrotherapy requires many components and results in products that are more difficult to apply and utilize. Application: The wearable disposable electrotherapy device is simply applied to the skin. The device is discreet and automatically initiates and provides a single dose. With conventional electrotherapy (images 820) a multi-step process involves tethering the electronic stimulator to the patient, using disposable electrodes, and programming/initiating therapy. Storage/disposal: The wearable disposable electrotherapy device minimizes environmental impact. Conventional electrotherapy devices have both durable electronics which must be stored and charged (e.g., batteries) between uses (and whose eventual disposal includes toxic materials) as well as disposal of single use electrodes (which in themselves have more metal than a wearable disposable electrotherapy).

Usability is a barrier to electrotherapy adoption and compliance. The described wearable disposable electrotherapy device does not require patients to be tethered to an electronic stimulator with numerous steps at each use and between each use. These obstacles complicate the treatment experience, throttle adoption and impair compliance with prescribed regimens of electrotherapy. The described wearable disposable electrotherapy device provides a categorical improvement in usability, for the first-time making electrotherapy use as simple as to pharmacotherapy or topical medication, by fundamentally simplifying dissemination, storage, use, and disposal (FIG. 8).

The wearable disposable electrotherapy device is more efficient than traditional electrotherapy. The wearable disposable electrotherapy device includes the smallest amount of materials necessary for delivering a single dose (e.g., minute quantities of functional and electrochemically active materials besides materials needed for interfaces). At the end of a treatment session, device materials are exhausted. In contrast, traditional devices consume energy inefficiently (e.g. displays, voltage step-up) using stand-alone batteries (with inevitable waste, Traditional electrotherapy systems are also expensive, being characterized by significant costs for materials, manufacturing (with higher Product Complexity Index), packaging, and shipment. They are also environmentally unfriendly, amassing hundreds of grams of molded plastics, PCBs, electronics, and connections. This burden of durable equipment still requires disposable electrodes (˜10 grams per use including metal connectors). By contrast, the wearable disposable electrotherapy device prioritizes scalable manufacturing processes that are air-stable and less energy-intensive, unlike costly material and assembly processes for electronic stimulators.

The production of traditional electrotherapy devices (electronics) hinges on the utilization of rare earth elements and heavy metals, alongside manufacturing processes that are resource-intensive and detrimental to the environment (toxins and carbon emissions). The end-of-life disposal of traditional devices further compounds their environmental footprint. Wearable disposable electrotherapy device production and use is inherently sustainable, and using only abundant and environmentally benign materials (Supplementary Table 1). Moreover, this unique form is coupled by design (FIG. 2) to scalable (additive) manufacturing techniques that do not depend on toxins, thus minimizing the environmental impact of the wearable disposable electrotherapy device. The limited usage (inevitable disposability) exacerbates the environmental impact of traditional devices.

Technology-centered health care advances (eg “smart” devices) often preferentially benefit users of privileged socioeconomic backgrounds. The wearable disposable electrotherapy device can decrease healthcare inequity. Conventional electrotherapies can have a high startup cost (all durable equipment), while wearable disposable electrotherapy can be trialed with a single disposable device. The multi-step setup, programming, and maintenance of traditional electrotherapy can be an accessibility barrier, unlike them auto-initiated bandage operation of the wearable disposable electrotherapy device. Deployability is a third factor for equitable access of medical devices, with wearable disposable electrotherapy not requiring batteries/charging and can be simply distributed.

### Battery Array Configurations

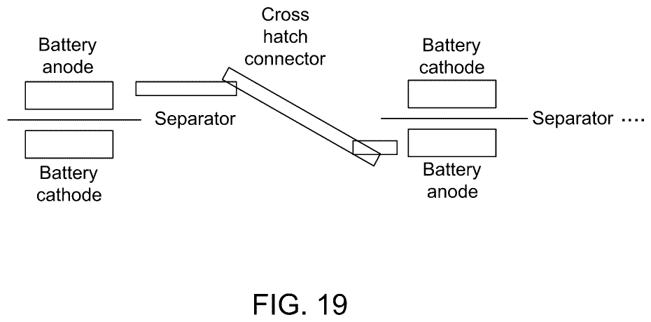

FIG. 19 illustrates a structure of battery pack with cross hatch connector, in accordance with some embodiments.

A battery cell can include an anode, a cathode, and a separator. Multiple battery cells forming a battery pack can be arranged in a coplanar structure where the anodes of at least two batteries are in the same device layers, or where the cathodes of at least two batteries are in the same device layers, or where the separator of at least two batteries are in the same device layers, or any combination thereof. In an implementation, the anodes and the cathodes and the separators of at least three batteries in series are each in the same layers (anodes in layer A, cathodes in layer B, and separators in layer C). In an implementation, the anodes and the cathodes of at least two battery cells in series are each in the opposite layers (battery 1 anode in layer A, battery 1 cathode in layer B, battery 2 anode in layer B, battery 2 cathode in layer A). This forms an alternating battery layer pattern. In one implementation, all batteries in a battery pack are alternating battery layers. In another implementation, the battery pack has an even number of battery cells. In another implementation, the battery pack has an odd number of battery cells. The design of even or odd numbers of battery packs is governed the configuration of the interface and associated discharge control.

Cross hatch connector supporting enhance battery packing and controlled discharge. The cross hatch connector can function as an interconnect that spans more than one device layer. Preferably the cross hatch connector connects an anode in one layer of one battery with the cathode of another battery in another layer. The cross hatch connector can connect an anode to an anode. The cross hatch connector can connect an anode to a cathode. The configuration of the cross hatch connector can be used to build of voltage across cells or drive specific electrochemical connections. The cross hatch connector can function as a switch or be otherwise adjustable. In one implementation there is an even number of battery cells in the battery pack where each battery pack is connected by a cross-hatch interconnect. In one implementation, the number of cross hatch connectors is one less than the number of battery cells. In one implementation, the number of cross hatch connectors is two less than the number of battery cells. In one implementation, there are at least two battery cells and only one cross hatch connector. The cross hatch connector can produce reversal in the net battery pack polarity compared to one surface of the enclosure. In one implementation, there are at least two battery cells and all anodes are in the same layer except for one anode that is in a distinct layer. In In a further implementation, the configuration includes an odd number of cross hatch connectors. In a still further implementation, the off number of cross hatch connectors is only one cross hatch connector. The cross hatch connector can produce reversal in the net battery pack polarity compared to the two stimulation electrodes or hydrogels.

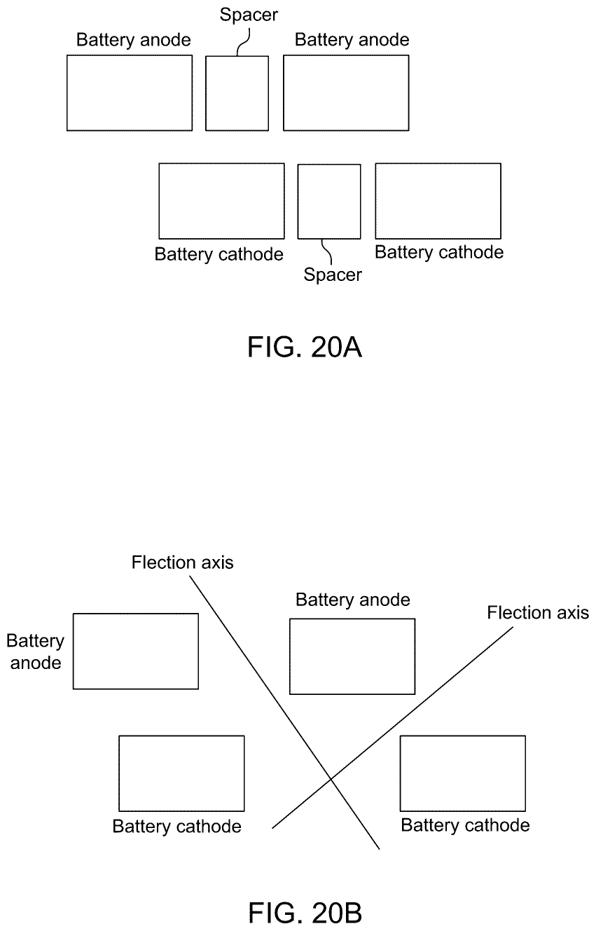

FIG. 20A illustrates a configuration of battery anodes with a spacer, in accordance with some implementations. FIG. 20B illustrates a configuration of battery anodes and cathodes with flexion axis, in accordance with some implementations.

The anode of one battery and the anode of another battery can be in at least one of the same layers. The anode of one battery and the anode of another battery entirely in the same layer or layers. A spacer is applied between the anodes where the spacer is in at least one layer with both the anodes. The spacer is configured to ensure controlled discharge.

The cathode of one battery and the cathode of another battery can be in at least one of the same layers. The cathode of one battery and the cathode of another battery can be entirely in the same layers or layer. A spacer is applied between the cathodes where the spacer is in at least one layer with both the anodes. The spacer is configured to ensure controlled discharge.

Referring to FIG. 20A, the described implementations can be combined such that at least four anodes and at least four cathodes and at least to spaces are positioned. The anodes can be positioned in row with one spacer. The cathodes can be positioned in row with one spacer. The anodes and cathode rows can be distinct rows. The anode in one row can be aligned with the cathode in the other row. The anode in one row can be misaligned with the cathode in the other row. When not aligned the overlap between an anode and a cathode can be 20%, 75%, or 90%. In one implementation all the anodes are cathodes have a similar alignment leading to a uniform alignment.

Referring to FIG. 20B, the described device includes a flection axis. Anodes and cathodes can be arranged with consideration of the arial geometry. In one implementation, all anodes can be in one row and all cathodes can be in another row. In another implementation, in each row anodes and cathodes alternate. The anodes and cathodes are aligned in such a manner that a flexion axis exists that is at an angle between the anode and the cathode. The angle of the flexion axis relative to the line formed a row of cells is greater than 10%, preferably greater than 30%. The angle is 30-60% in an implementation. When a battery pack contains at least two cells (at least two anodes and two cathodes) multiple flection axis is designed. With at least two flection axis the axis can intersect. The intersection of flection axis lies in a position not occupied by a battery cell. In an implementation, the number of flection axis is the number of battery cells. In an implementation, the number of flection axis is the sum of the number of anodes and cathodes. In an implementation, the number of flection axis is double number of battery cells, or double number of battery cells plus one, or double number of battery cells minor one. In a further implementation, at least two of the flection of axis intersect. In a still further implementation, the intersection is at an angle of approximately 90°. In a still further implementation, the intersection is at an angle of approximately 60°. The flection axis can be enabled by cuts as described. The flexion axis can be enabled by modification of the stiffness of material near the flection axis as explained. The formulation of the flection axis is based on the desired form and bending of the device while limiting strain on the battery packs. A battery pack can include at least 5 cells and at least 9 flection axes. A battery pack can include at least 8 cells and at least 12 flection axis. The minimum distance between a battery cell in one row and a battery cell in another row, where there is a flection axis between the cells is 5 mm, preferably 3 mm, still more preferably 0.5 mm. A spacer can be combined a flection axis when the space provides appropriate mechanical support.

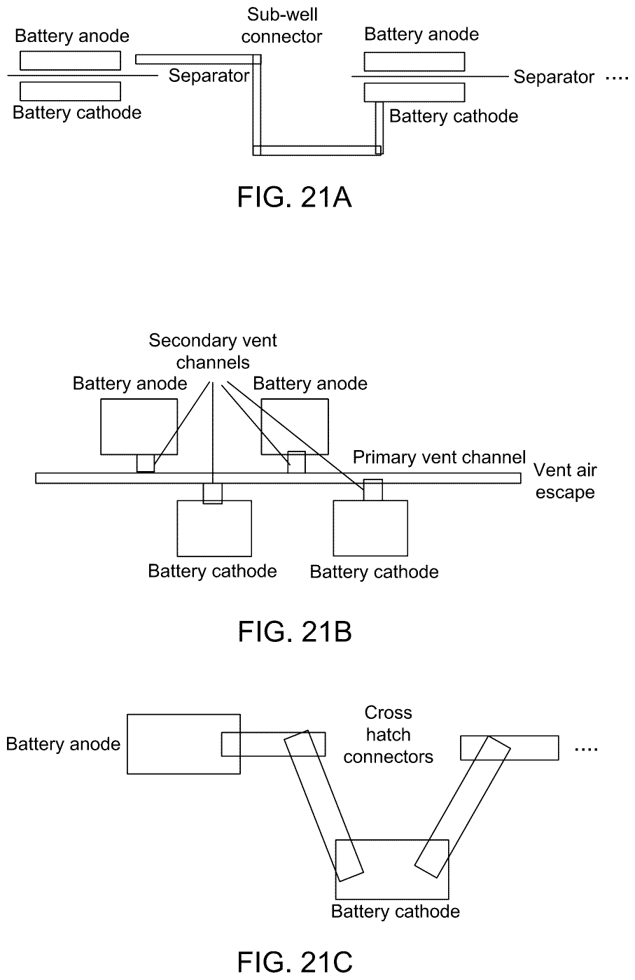

FIG. 21A illustrates a configuration of a battery pack with a sub well connector, in accordance with some implementations. FIG. 21B illustrates a configuration of a battery pack with secondary vent channels, in accordance with some implementations. FIG. 21C illustrates a configuration of a battery pack with cross hatch connector, in accordance with some implementations.

Referring to FIG. 21A, cross hatch connector is designed to span multiple layers where at least some of those layers is not used by the anode or cathode the cross hatch connector is connecting. This is a sub-well type of cross hatch connector (“sub well connector”). A sub well connector can connect to the enclosure. The sub well connector can be modified by pressure applied to the enclosure. In an implementation, a battery pack of at least two batteries has only one sub well connector.

Referring to FIG. 21B, second vent channels vent air from battery cells to a primary vent channel. The primary vent channel includes at least one end that is a vent to the environment. The primary vent channel is greater than 0.002 mm, or greater than 0.0001, or greater than 0.1 mm. The secondary vent channels are less than 0.01 mm or less than 0.0001 mm or less than 0.00005 mm. In an implementation there is one secondary vent channel for each battery. In an implementation there is one secondary vent channel for each anode or cathode. Each secondary cent channel is connected to at least one primary vent channel.

Referring to FIG. 21C, a cross hatch connector can be configured along the arial configuration of the battery pack. An anode and a cathode can be in distinct position in the arial (top view) perspective. A cross hatch connector connects the anode and the cathode. At least two cross hatch connectors can be applied in a battery pack. The cross hatch connector can include at least two elements where one element is in one layer and the other element is in multiple layers. In one implementation, an anode is near one surface of the device and a cathode is near another surface of the device and anode and cathode are connected by a cross hatch connector. The cross hatch connector can be a total length of 0.1 mm, 0.5 mm, 1 mm, 5 mm, 10, mm. The total distance spanned across layers by a cross hatch connector can be 3 mm, 1 mm, 0.5 mm, 0.2 mm, 0.05 mm, or 0.01 mm. The geometry and size of the cross action governed by the associated battery back configurations. The total area of the cross hatch connector can be 2%, 10%, 20%, 50%, 75%, or 150% of the area of an electrode the cross hatch connector is connected to. In one implementation at least three batteries are connected by at least two cross hatch connectors. In a further implementation, one cross hatch connector is in the same layers as the anodes and cathodes and the second cross hatch connector is in at least some layers with anodes and cathodes. In a further implementation, the second cross hatch connector is in at least some layers with anodes and cathodes has a length greater than 1 mm. In an implementation there are 3 batteries and the total area of a cross hatch connector is 20% on a battery cell connected to the cross hatch connector.

Manufacturing processes for the device are described and should not be construed to limit any of this disclosure.

Substrate preparation. The substrate is fabricated from 100 μm-thick PET sheets (McMaster #8567K44) which undergo a series of preparation steps: 1) Heat treating of the substrate for the purpose of mechanical stability by baking at 90° C. for 15 min; 2) Laser cutting to perforate boundaries of the device and folding line as well as throughholes. This allows the pouch to be a part of the substrate sheet during printing steps, and subsequently to be separated for sealing of the battery pouch. The fold line facilitates accurate alignment of battery electrodes during folding while preventing easy tearing; 3) Washing with isopropyl alcohol followed by air drying; 4) Surface treatment with cold plasma (Relyon plasma piezo brush PZ3) to improve adhesion.

Printing of conductive tracks (interconnect, cross hatch connector, sub well connector, through-holes, stimulation electrodes, current collectors). A copper conductive ink (copprint LF-350) or silver conductive ink (Saral Silver 700) is screen printed (Novastar SPR-45 stencil/screen printer) onto both sides of the substrate (device top view 1010, device bottom view 1030). Following printing, the substrate is heated in an oven (90 C) to facilitate the evaporation of the solvents present in the ink used. After baking, the thickness of the printed silver track is ˜20 μm. Subsequently, a carbon passivation layer (Saral Carbon 700A) is screen printed and baked in a similar fashion; this was only on the inner side with a screen template one mm wider than that used for silver from each side. After baking, the thickness of the printed carbon track is ˜40 μm.

To establish an electrical connection between both sides of the substrate, the process implements laser-cutting to create micrometer-sized holes in the form of an array in the substrate before printing. During the screen printing of the conductive inks on both sides of the substrate, the ink permeates these holes, creating a conductive path between the two sides of the substrate. The size and number of these holes are tailored to specific ink properties (rheology and particle size) to achieve reliable adhesion and conductivity. To support device verification and validation, three conductive tabs can be added to the design at the battery pack terminals and at the cathode stimulation electrode, which allow monitoring of current and voltage.

The device includes preparation of cathode ink, anode ink and electrolyte. The cathode ink used for one exemplary device is composed of 70 wt % electrolytic manganese dioxide (EMD), 3.5 wt % carbon black as a conductive additive, 5 wt % KOH, 20 wt % deoxygenated DDI water, and 1.5 wt % PVA (average MW 94 k) as a binder. The slurry is prepared by mixing water and PVA and KOH in a nitrogen box to prevent oxygen dissolution in water during mixing. This solution then is sealed and kept in the refrigerator at 4° C. Prior to use, the mix of EMD powder and carbon black powder is added and mixed and finally loaded into a syringe for application. The cathode ink for applications is composed of 79.5 wt % electrolytic manganese dioxide (EMD), 3.5 wt % carbon black as a conductive additive, 15.5 wt % deoxygenated DDI water, and 1.5 wt % SBR as a binder.

The anode ink used for exemplary device consists of 75 wt % zinc powder, 0.4 wt % zinc oxide as corrosion inhibitor, 5 wt % KOH, 17.8 wt % deoxygenated deionized (DDI) water, 0.25 wt % PAA (average MW 450 k), and 1.55 wt % sodium carboxymethylcellulose (Na-CMC) (average MW 90 k) as binders. The slurry is prepared by mixing all ingredients, except for zinc powder, in a nitrogen box to prevent oxygen dissolution. The slurry is then sealed and refrigerated at 4° C. Prior to use, zinc powder is added to the solution inside of a syringe and mixed to create the anode ink. The anode ink for applications is composed of 74 wt % zinc powder, 0.3 wt % zinc oxide as corrosion inhibitor, 23.5 wt % deoxygenated DDI water, 1.4 wt % Na-CMC as filler, and 0.8 wt % styrene-butadiene rubber (SBR) as binder.

The electrolyte is formulated with 66 wt % deoxygenated DDI water and 34 wt % KOH.

The device includes a sealing method. The device is sealed using a double-sided acrylic adhesive designed for low surface energy plastics, with an interior carrier film for enhanced mechanical stability with a thickness (0.17 mm) less than the battery pack cells and interconnects (3M 9495LE). The double-sided adhesive sheet is cut (prior to removing the liner of both sides) using a laser cutter into two strips, each with openings corresponding to cells on one row of the battery pack (1160). When placed on the substrate a gap between the two adhesive strips forms the central channel of the venting system (1040, 1160).

To implement the placement of separator, the separator membrane (Celgard 5550) used consists of polypropylene film laminated to a polypropylene nonwoven fabric, coated with hydrophilic surfactant for aqueous applications. This membrane has a thickness of 110 μm and 55% porosity, for high electrolyte retention and ion conductivity for high discharge rate. The membrane is laser cut to appropriate size and placed on the cell using cut double sided tape.

The device includes vent channel and valves. After placing the membranes on double sided tape, thin strips are printed on the adhesive using non-stick ink (1 mm width), connecting the middle of the battery cell to the vent channel (1040, 1130). These thin strips mask the adhesive, creating normally closed valves, they provide an escape for air (during battery pack sealing) or generated hydrogen by the cells, to the vent channel.

The device includes printing of active materials on current collectors. In the exemplary device each membrane is saturated with 9.5 mg (8 μL) of electrolyte before printing active materials. The volume of electrolyte is crucial to control since insufficient electrolyte reduces battery performance, while excess electrolyte wets the surface of double-sided tape resulting in poor sealing. Then anode and cathode inks are deposited on printed substrates using screen/stencil. Immediately after printing anode ink, double sided tapes with soaked membranes are placed on two anode rows of the device. This prevents printed zinc from drying. By folding the device on its fold line, both sides of the substrate meet in alignment to form a sealed battery pack (device top view 1010). The sealed battery pack goes through a roller from each side to the middle of the device where the vent channel is to push trapped air out of cells through valves.

To implement the battery pack quality control, after compressing the pack, terminals of the pack are connected to a multimeter to read the initial open circuit voltage (IOCV). Some manufacturing problems can be detected by observing subtle changes of voltage.

To implement the interface hydrogel application, ion conductive hydrogels (Axelgaard AG625) are cut to the size (45 mm×56 mm, 25.2 cm²) and placed on the stimulation electrodes. As needed, fastening (non-conductive) hydrogel (Axelgaard AG535) can be used in between stimulation electrodes. The hydrogels are covered with PET liner until use. The hydrogel placement step was omitted for battery pack discharge verification tests. The optimization of hydrogel (and associated stimulation electrode material) for the exemplary application followed protocols developed to screen for a) tolerability; b) skin irritation; c) impedance (compliance voltage); and d) material/mechanical properties.

Electrical performance verification of cell/battery packs under constant current discharge was conducted using a sourcemeter (Keithley 2450 SMU). Electrochemical impedance spectroscopy (EIS) experiments were conducted with a Prinston Applied VersaSTAT 4 Potentiostat/Galvanostat. AC impedance measurements were performed potentiostatically at open circuit voltage and a small signal stimulus of 5 mV within a frequency range of 10 mHz-10 kHz. The test is conducted with 60 data points distributed across the frequency range on a logarithmic scale. The battery model components were selected based on comparable electrochemistry analysis and fit to EIS data (AMETEK, ZView). Model parameters can reflect mass transfer, chemical kinetics, and electrical resistance of current collectors and conductive additives in electrodes, from which device chemistry (particle size, electrode porosity) and structure (thickness of electrode, compaction) can be refined. This includes the upper limit on current density limited by mass transfer of charge carriers. SEM images recorded using SUPRA 55, with an acceleration voltage of 16 kV and backscattered electron detector.

### Load Characterization and Validation.

The study was conducted in accordance to protocols and procedures approved by the Institutional Review Board of the City College of New York. All volunteer participants provided written informed consent to participate in the study. The study included 17 subjects (15 male) between the ages of 19 and 38 (M=25, SD=±6.4).

For one series of tests, participants were excluded if they presented with any skin disorder at or near stimulation locations that compromised skin integrity, such as eczema, rashes, blisters, open wounds, burns including sunburns, cuts, or other skin defects, as the goal of this study was not to determine if skin impairments influence the tolerability or to access electrical stimulation to enhance wound healing.

For one series of tests, stimulation was applied to the ventral or dorsal side of the subject's left or right forearm. No more than one dose was applied to a region per day (e.g. ventral surface of right arm). All devices were at room temperature (22° C.) immediately prior to testing.

For load characterization tests, devices with the same design as wearable disposable electrotherapy but with only interface components and without deposition of active materials were made. Prior to the test, subjects' forearms were cleansed with soap and water and then dried. After placement of ion conductive hydrogels the monitoring tabs were connected to a sourcemeter (Keithley 2450 SMU). According to the test, the sourcemeter output was set to either constant voltage with a peak current limit, or to constant current with a voltage compliance limit. Output was enabled before placement of the test device on skin.

Temperature was recorded using three thermocouple probes placed in the empty battery pouch, one over the anode stimulation electrode, one over the cathode stimulation electrode and one in the middle of the device over fastening hydrogel. Photographs were taken immediately before and after stimulation under consistent lighting conditions and skin temperature was recorded using a thermal camera (FLIR One Pro). At the beginning and during stimulation, subjects reported subjective pain on a VAS scale every 2 min. Approximately 24 hours after stimulation, subjects' skin was evaluated for any enduring skin irritation.

The validation procedure was like load tests using the interface test device; instead of connecting the device to the sourcemeter, the Wearable Disposable Electrotherapy Device with active batteries was used. To record the voltage and current of the device, a custom high impedance analog interface was used (0.4 attention factor), a 100Ω series resistor (for current), and acquisition system (DATAQ DI-1100).

FEM Device Simulations are used. For the exemplary device, the experiment developed a computer-aided design (CAD) model of the wearable disposable electrotherapy device prototype and underlying superficial tissue. The biophysical and thermo-electrical properties of biological tissues were based on previous studies and heat-transfer biophysics followed standard assumptions and methods. An approximate temperature distribution throughout a perfused tissue can be found by solving the bio-heat transfer (Pennes) equation. For the thermal boundary conditions, all external boundaries were insulated except the top surface which was assigned heat flux. For the tDCS device, a previously detailed and verified MRI-derived (1 mm³ T1/T2; 36 year old male) model was used. For the iontophoresis device model and the wound healing device model a forearm/hand model was developed from a high-resolution MRI (1.25 mm³ T1/T2/Petra; 33 year old male).

CAD structures (devices) were modeled in SolidWorks 2022 (Dassault Systemes Corp., MA, USA) and Simpleware ScanIP U-2022.12-SP2 (Synopsys, WA, USA) imported and numerically solved in COMSOL Multiphysics 5.6 (COMSOL Multiphysics, Boston, MA) under conventional parameters and quasi-static physics (46). The resulting finite element model comprised >32,690,000 tetrahedral elements (>11,120,000 degrees of freedom, with 30 s time-steps) for the exemplary device, >1,880,000 tetrahedral elements (>2,590,000 degrees of freedom) for tDCS model, >50,000,000 tetrahedral elements (>72,000,000 degrees of freedom) for the iontophoresis model, and >35,000,000 tetrahedral elements (>54,000,000 degrees of freedom) for the wound healing model.

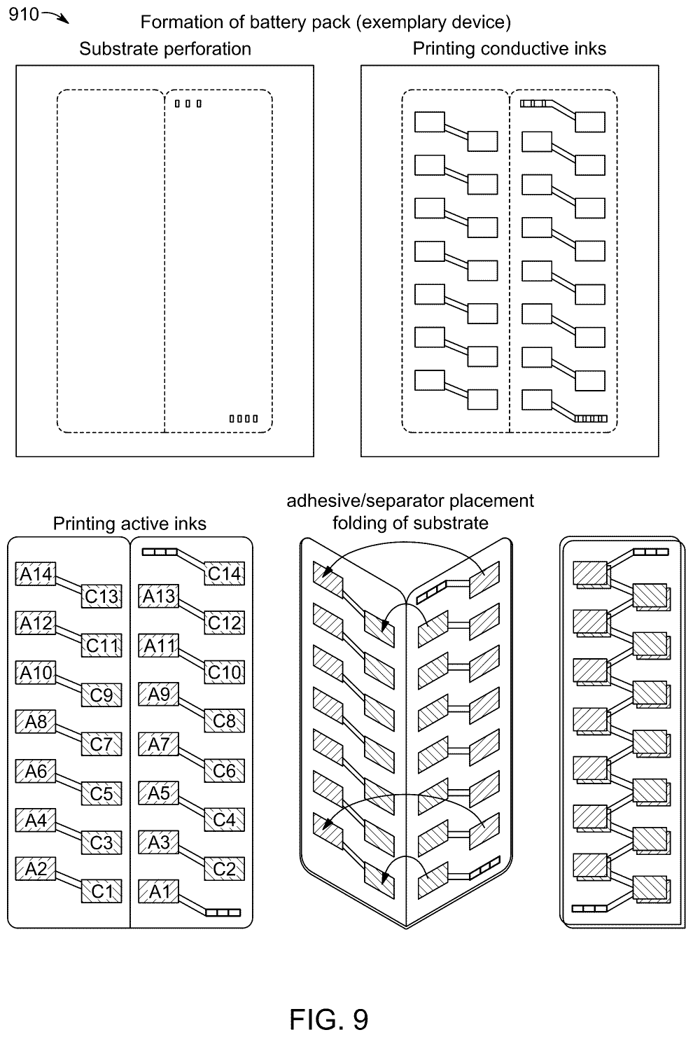

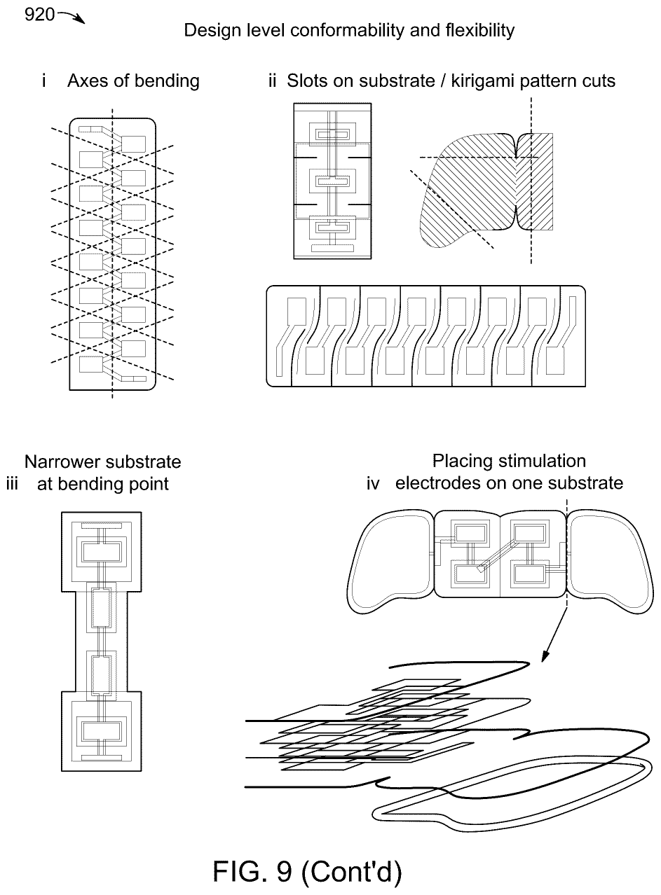

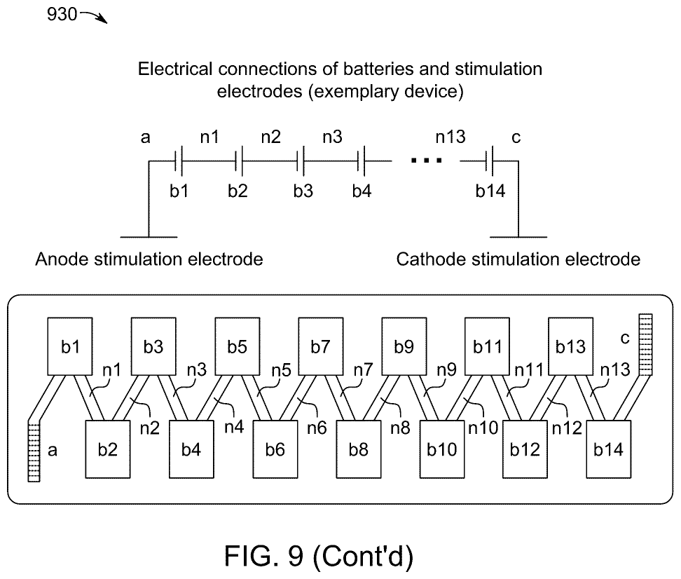

FIG. 9 illustrates a formation of 3D structure and battery pack functionality of the wearable disposable electrotherapy device, in accordance with some implementations. in 910, all components, including interconnect and active materials (forming cell pairs, numbered), can be printed on a single substrate. In the final manufacturing steps separators and adhesive are added and the device folded, whereby battery cells are formed and connected. in 920, per application specific requirements, geometry designs further support conformability along low flexural rigidity axes (i), using pattern cuts (ii), and variations in device width (iii) or thickness (iv). In configuration 930, the battery pack output at the terminals (a: stimulation anode, c: stimulation cathode) reflects the series output of battery cells (b1 to b14) through interconnects (n1 to n13).

FIG. 10 illustrates a geometry of exemplary wearable disposable electrotherapy device (battery pack structure and interconnects, interfaces, venting system), in accordance with some implementations. A device top view 1010 shows electrochemical architecture of series cells leading to battery pack terminals (with interconnects to bottom substrate), and integrated venting system. Device cross sections 1020 at battery cell (Cs1), at through-holes (Cs3) and at middle of device (Cs2) show materials and vent channel. A device bottom view 1030 shows interface components with through-hole interconnects (translayer interconnects) to top substrate. Device cross section 1040 illustrates the device venting system.

FIG. 11 illustrates a 3D geometry of exemplary wearable disposable electrotherapy device, in accordance with some implementations. Diagram 1110 shows exploded view of device with inset 1120 showing array of holes on the substrate filled with conductive material, connecting stimulation electrode on back of the substrate to the interconnect on the top of the substrate. Diagram 1130 shows aerial geometry showing the venting system. The vent channel (negative space) is shown in blue and normally closed valves are shown in purple. Diagram 1140 shows an exploded view of layers of one battery cell (top to bottom: substrate enclosure, silver conductor, carbon current collector, anode, separator, cathode, carbon current collector, silver conductor. Diagram 1150 shows a view of two interconnects (one on bottom and one on top substrates) connecting one battery cell to two adjacent cells. Diagram 1160 shows two strips of double-sided tape with cutouts for battery cells, and narrow strips of adhesive masking. The one mm gap between adhesives forms the vent channel. Diagram 1170 shows an exploded device view with indexed design elements.

FIG. 12 illustrates single battery cell designs (time/current self-limited). Verification 1210 shows a design of single packaged cell with interconnects for battery discharge and electrochemical impedance tests. Diagram 1220 shows an exploded view of layers of single cell test device. Photograph 1230 shows a of a single cell test device. Diagram 1240 shows an experimental test setup for a single cell test device discharge test. Plot 1250 shows discharge curves for single cells under 700 μA constant current. The flat discharge curve followed by sudden drop in anode limited battery against discharge curve of cathode limited battery. In anode limited batteries, total capacity of the battery is controlled by the mass of zinc. Plot 1260 shows discharge curve of a printed battery beyond 0.9 v to predict interaction with load. Plot 1270 shows discharge curves for single cells connected to 33Ω constant load. High current (peak and average) during discharge resulted from a typical cell. Flat discharge and low current rate for a cell with similar mass of active materials with additional current limiting mechanism. Note maximum discharge current can be self-limited independent from load value. Plot 1280 shows experimental test setup for a single cell test device electrochemical impedance spectroscopy (EIS). Plot 1290 shows a Nyquist and Plot 1295 shows a Bode plot of electrochemical impedance spectroscopy performed potentiostatically at open circuit voltage with a small signal stimulus of 5 mV across 10 mHz-10 KHz frequency range.

FIG. 13 illustrates subject-wise simulation of exemplary wearable disposable electrotherapy device performance. Plots 1310 show an interface-physiological load test results for 4 subjects under constant current stimulation at amplitudes of 1-6 mA and voltage limit of 22.4 v during 20 min. Plots 1320 show an associated isotemporal V-I profile of subject-specific interface-physiological load results (colored solid lines) are overlaid with battery pack V-I performance (colored dashed lines). Battery pack data is from separately-collected galvanostatic discharge tests (1-6 mA, 20 min). The overlaid interface-physiological load and battery pack V-I profiles are represented for varied time points during discharge (colors). At each timepoint, the intersection of these V-I plots reflects source-load coupling (see theory). The connection of these intersections (solid black line) is then the simulated device discharge trajectory. Plots 1330 show a predicted discharge trajectory is shown as current over time performance. On a subject-wise basis, this illustrates the novel design/simulation approach developed for wearable disposable electrotherapy devices and is then validated.

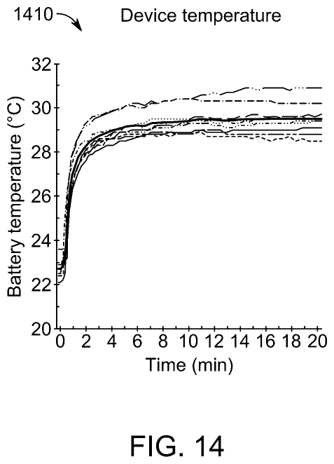

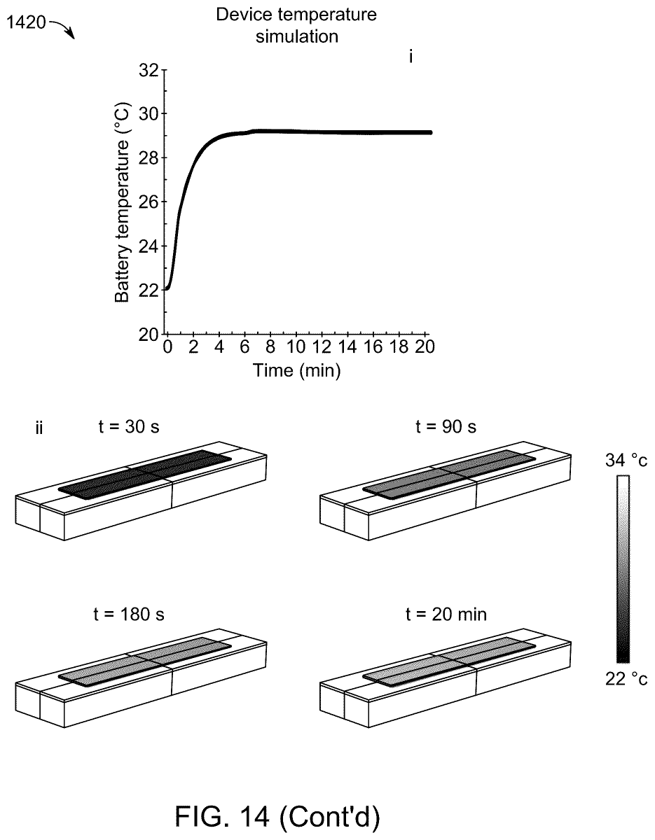

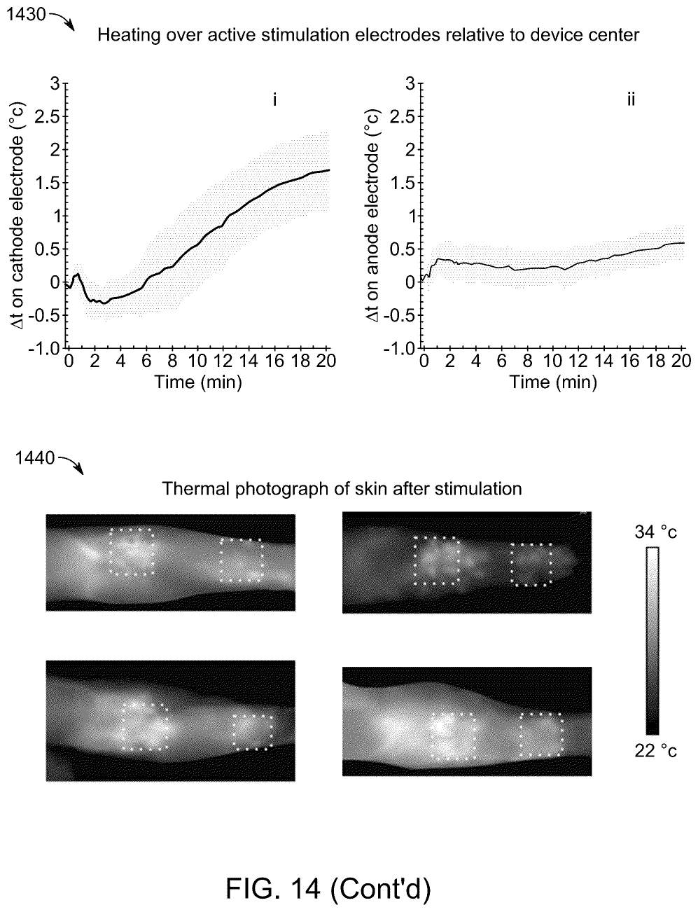

FIG. 14 illustrates temperature transition of exemplary wearable disposable electrotherapy device, in accordance with some implementations. Device temperature quickly warms to skin temperature by placement on subjects' skin (t=0). Plot 1410 illustrates battery pack temperature measured using sensors in place of battery active material in interface test devices for 10 subjects (colored lines) and average (black line). Plot 1420 illustrates FEM bioheat simulation of battery pack temperature. Plot 1430 illustrates elative heating of battery material over stimulation cathode electrode (i) and over stimulation anode electrode (ii), normalized to temperature in the center of device (solid lines: average; shaded areas: variance). Plot 1440 illustrates IR thermography of skin after stimulation showing relative heating of skin under cathode (left) and anode (right) stimulation electrodes.

FIG. 15 illustrates performance of iontophoresis application wearable disposable electrotherapy device on a skin phantom. Diagram 1510 shows experimental set-up to evaluate enhanced ionic delivery. Diagram 1520, top row shows passive diffusion of dyes into phantom placed on the electrodes of a control device (devoid of battery materials). The bottom row shows enhanced dye movement due to iontophoresis, demonstrating the device's efficacy in facilitating active transport of charged molecules. 0.8 ml of 0.14% Phenol Red (negatively charged, molecular weight 354.4 g/mol) was applied to the nonwoven felt placed on the negative stimulation electrode, and 0.8 ml of 0.2% Methylene Blue (positively charged, molecular weight 319.9 g/mol) was applied to positive stimulation electrode. The phantom blocks were prepared using 250 g of deionized water, 2.5 g of agar powder, and 250 μg of NaCl. The blocks were placed on both the control and active devices for two hours, then sliced and placed on a grid with one mm spacing between lines to visualize dye diffusion.

SUPPLEMENTARY TABLE 1
Material use of Wearable Disposable Electrotherapy
vs. conventional electrotherapy consumables.

| | | | | | | | | | Consumable components of | | | |
|---|---|---|---|---|---|---|---|---|---|---|---|---|
| | Wearable Disposable Electrotherapy | | | | | | | | conventional electrotherapy | | | |
| | | | | | | | Wound | | AA | | Disposable | |
| Material | Exemplary | | tDCS | | iontophoresis | | healing | | battery | | electrode (2) | |
| Conductive | 4.05 | g | 3.96 | g | — | | 1.2 | g | | | ~4 | g |
| Hydrogel | | | | | | | | | | | | |
| Nonwoven/ | — | | — | | 0.56 | g | — | | | | 0.5-2 | g |
| sponge | | | | | | | | | | | | |
| Plastic | 2.80 | g | 1.93 | g | 1.24 | g | 0.35 | g | | | ~4 | g |
| substrate | | | | | | | | | | | | |
| Adhesive | 1.885 | g | 888 | mg | 983 | mg | 340 | mg | | | ~1.5 | g |
| Separator | 111 | mg | 45 | mg | 40 | mg | 35 | mg | 600 | mg | | |
| Electrolyte | 196 | μl | 150 | μl | 152 | μl | 42 | μl | | | | |
| Copper ink¹ | 76.1 | mg | 22.6 | mg | 37.8 | mg | 11.1 | mg | | | | |
| Carbon ink¹ | 122.3 | mg | 38.2 | mg | 25.8 | mg | 7.8 | mg | | | | |
| Anode | 373.8 | mg | 170.9 | mg | 142.4 | mg | 27.7 | mg | 5.2 | g | | |
| material¹ | | | | | | | | | | | | |
| Cathode | 756 | mg | 345.6 | mg | 288 | mg | 56.2 | mg | 12.3 | g | | |
| material¹ | | | | | | | | | | | | |
| Solid Metal | — | | — | | — | | — | | 4.6 | g | 0.6-2 | g |
| parts | | | | | | | | | | | | |

According to isotemporal trajectory theoretical framework several steps are taken, First, the wearable disposable electrotherapy device performance is characterized (considered a coupled system of battery pack with interfaces-physiological load) using an approximation of uncoupled parts. The analyses are based on on the measured behavior of each part (battery and interfaces-physiological load) independently. These results explain the unique current regulation theory of Wearable Disposable Electrotherapy, as well as the specific simulation step used (Section 4.0) as part of the device design process (FIG. 2).

Battery pack and interfaces-physiological load as uncoupled dynamical systems is characterized. The device design approach identifies and characterizes two subsystems: battery pack (B) and interfaces-physiological load (L). First, the approach considers these two subsystems independently (i.e. without interactions). The voltages V_{B} and V_{L} of B and L, respectively, are response functions of time t:

$$\begin{matrix}{V}_{B} ( t ) = {f}_{B} ( {I}_{n} , t \vert {α}_{B} , {e}_{B} ) , \\ {V}_{L} ( t ) = {f}_{L} ( {I}_{n} , t \vert {α}_{L} , {e}_{L} ) ,\end{matrix} \qquad ( SA . I )$$

where Iₙ is a constant current stimulus; α_{B} and α_{L} are internal parameters of each system; while e_{B} and e_{L} are environmental factors affecting each subsystem. These parameters also have a temporal dependency.

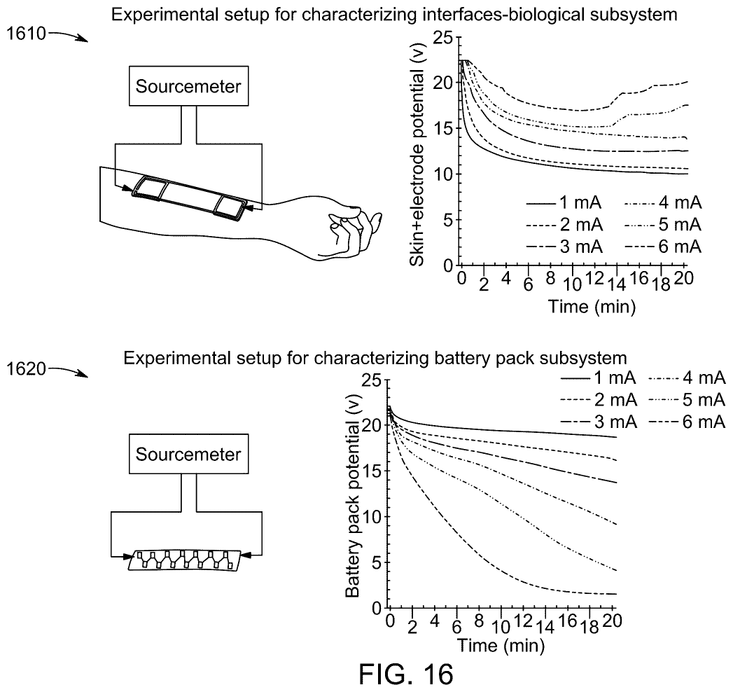

FIG. 16 shows that, experimentally, by independently connecting the battery packs (1620) or interfaces-physiological load (1610) to a source meter, with varied applied constant current levels (e.g. Iₙ=one mA, 2 mA . . . ), and measuring the associated output voltage (V_{B}, V_{L}), the response functions of each system are determined (i.e. f_{B} and f_{L}).

A next step is battery pack and interfaces-physiological load as a coupled dynamical system. In device design two subsystems: battery pack (B) and interfaces-physiological load (L) are in fact physically coupled. Then, system B-L is composed by the subsystems B and L such that both parts exchange the same current I(t) and have equal voltage V (t). The voltage of each part of the system evolved with the equations:

$$\begin{matrix}{\hat{V}}_{B} ( t ) = {\hat{V}}_{B} ( I ( t ) , t \vert {α}_{B} , {e}_{B} ) , \\ {\hat{V}}_{L} ( t ) = {\hat{V}}_{L} ( I ( t ) , t \vert {α}_{L} , {e}_{L} ) ,\end{matrix} \qquad ( SA . II )$$

$$and$$

$${\hat{V}}_{B} ( t ) = {\hat{V}}_{L} ( t ) .$$

A next step In the solution approximation of \hat{V}_{B}(t) and \hat{V}_{L}(t) from uncoupled subsystems. Given only independently characterized subsystems (the battery pack and interfaces-physiological load) the goal is to simulate the performance of the coupled system (e.g., the performance of the device when applied to the body).

First, the simulation expands (SA.II) to the first-order Taylor series:

$$\begin{matrix}{\hat{V}}_{B} ( t ) \approx {\hat{V}}_{B} ( {I}_{n} , t \vert {α}_{B} , {e}_{B} ) + \frac{\partial {\hat{V}}_{B}}{\partial I} ( {I}_{n} , t ) ( I - {I}_{n} ) \\ {\hat{V}}_{L} ( t ) \approx {\hat{V}}_{L} ( {I}_{n} , t \vert {α}_{L} , {e}_{L} ) + \frac{\partial {\hat{V}}_{L}}{\partial I} ( {I}_{n} , t ) ( I - {I}_{n} ) .\end{matrix}$$

where Iₙ is a fixed value of I(t). We denote to any time value such as I(t₀)=Iₙ.

The condition \hat{V}_{B}(t)=\hat{V}_{L}(t) is verified if and only if

$${\hat{V}}_{B} ( {I}_{n} , t \vert {α}_{B} , {e}_{B} ) + \frac{\partial {\hat{V}}_{B}}{\partial I} ( {I}_{n} , t ) ( I - {I}_{n} ) \approx {\hat{V}}_{L} ( {I}_{n} , t \vert {α}_{L} , {e}_{L} ) + \frac{\partial {\hat{V}}_{L}}{\partial I} ( {I}_{n} , t ) ( I - {I}_{n} ) ,$$

when |I−Iₙ| is small.

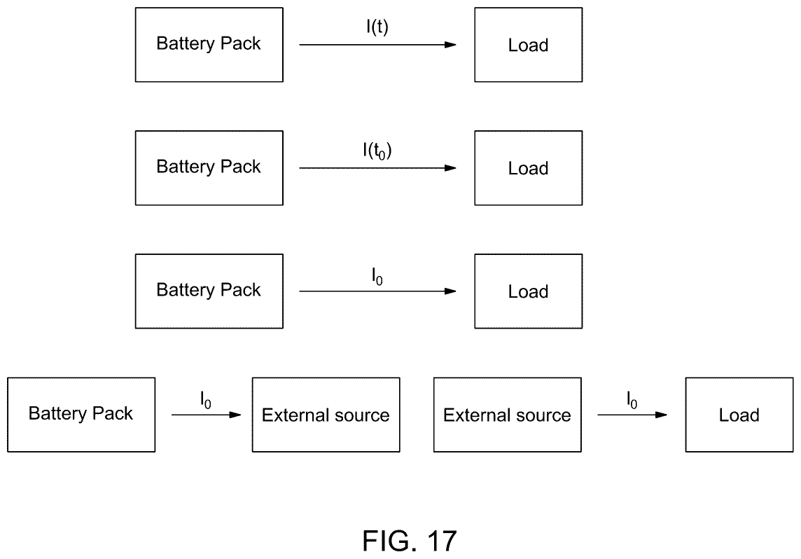

In the coupled system (FIG. 17), the subsystems exchange I(t). At time to, the value of I(t₀) is Iₙ. At this particular instant, systems B and L can receive I₀ independently (e.g., as if they were two uncoupled systems).

Then,

$$\begin{matrix}{\hat{V}}_{B} ( {I}_{n} , {t}_{0} \vert {α}_{B} , {e}_{B} ) = {f}_{B} ( {I}_{n} , {t}_{0} \vert {α}_{B} , {e}_{B} ) , \\ {\hat{V}}_{L} ( {I}_{n} , {t}_{0} \vert {α}_{L} , {e}_{L} ) = {f}_{L} ( {I}_{n} , {t}_{0} \vert {α}_{L} , {e}_{L} ) .\end{matrix}$$

In summary,

$${\hat{V}}_{B} ( t ) = {\hat{V}}_{L} ( t ) ⇔ {f}_{B} ( {I}_{n} , t \vert {α}_{B} , {e}_{B} ) + \frac{\partial {\hat{V}}_{B}}{\partial I} ( {I}_{n} , t ) ( I - {I}_{n} ) \approx {f}_{L} ( {I}_{n} , t \vert {α}_{L} , {e}_{L} ) + \frac{\partial {\hat{V}}_{L}}{\partial I} ( {I}_{n} , t ) ( I - {I}_{n} )$$

Moreover, since |I−Iₙ|) is small, it can be reduced to

$${\hat{V}}_{B} ( t ) = {\hat{V}}_{L} ( t ) ⇔ {f}_{B} ( {I}_{n} , t \vert {α}_{B} , {e}_{B} ) \approx {f}_{L} ( {I}_{n} , t \vert {α}_{L} , {e}_{L} ) . \qquad ( Approximate solution )$$

Therefore the approximate solution to the coupled system is described by the response functions under fixed constant current of the uncoupled system. Indeed, these response functions are known (from 2.0), It can be assumed the environmental dependencies (ep and e) are comparable under experiment-isolated system testing and couple system discharge.)

One can approximately solve the coupled system (B-L) performance when a device is applied to the body—by calculating the intersection of the response functions f_{B} and f_{L}—which are measured as independent subsystems (B and L):

$${f}_{B} ( {I}_{n} , t \vert {α}_{B} , {e}_{B} ) = {f}_{L} ( {I}_{n} , t \vert {α}_{L} , {e}_{L} ) or {V}_{B} ( t ) = {V}_{L} ( t ) . \qquad ( SA . III )$$

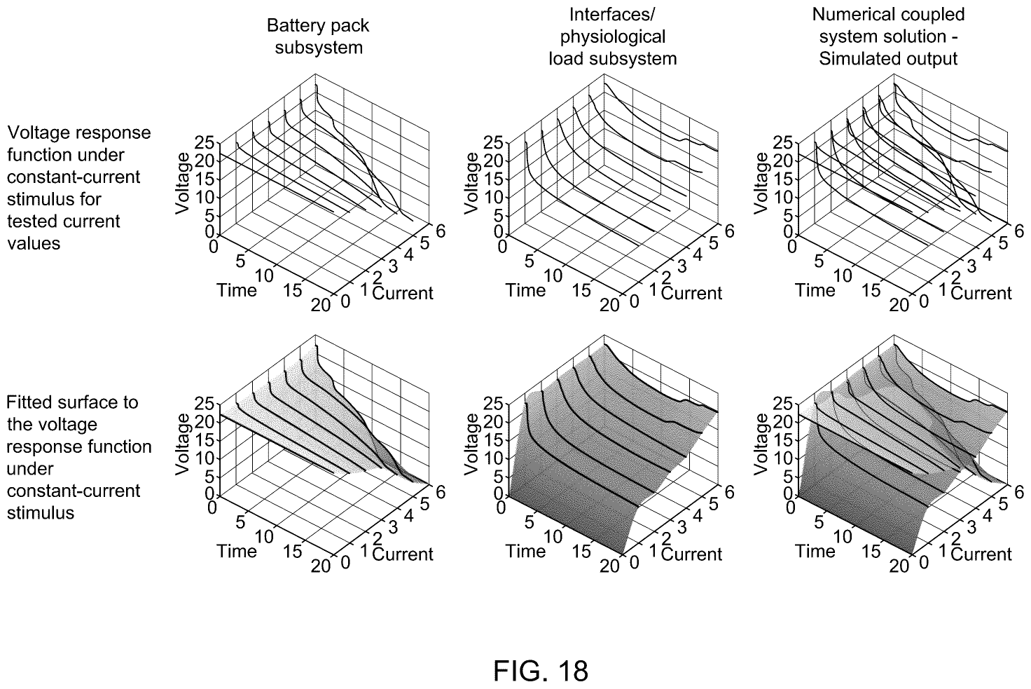

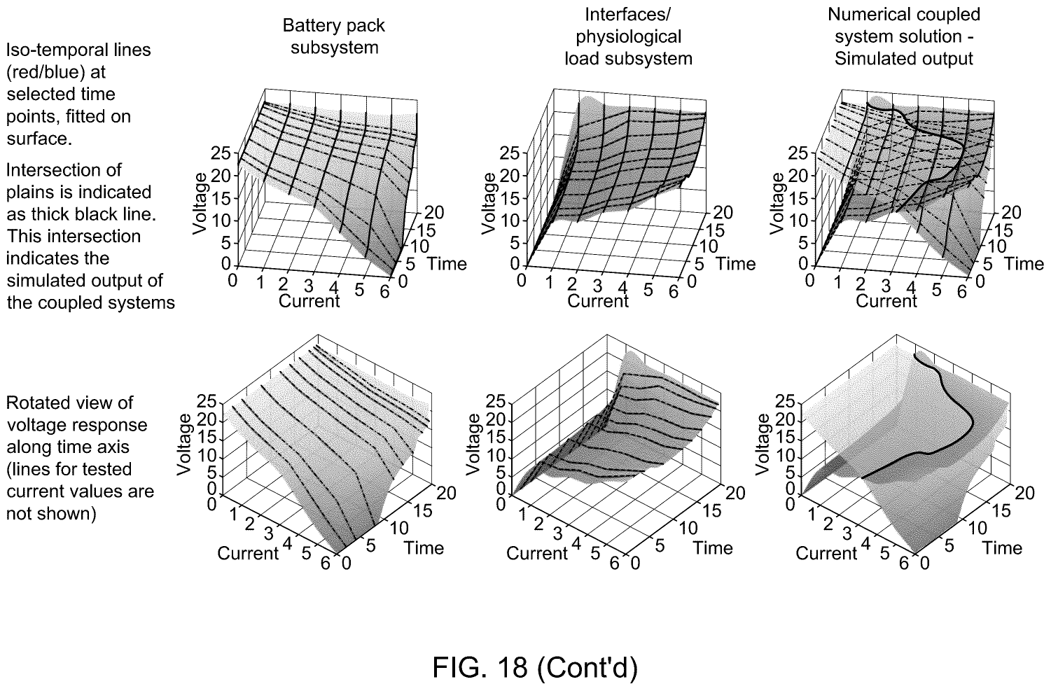

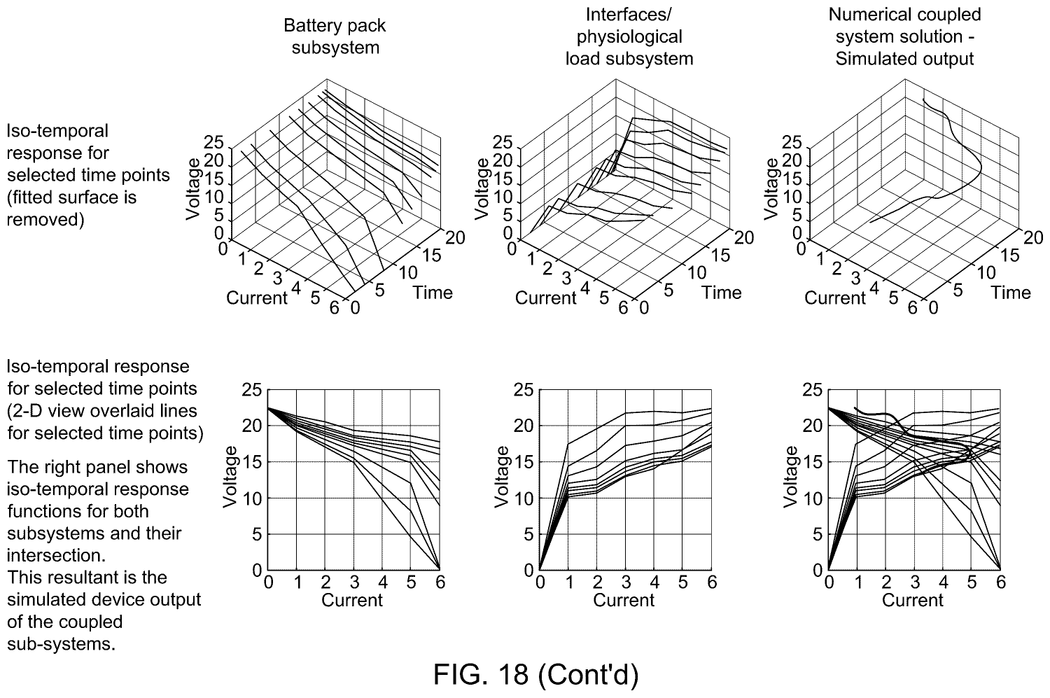

FIG. 18 shows a numerical implementation of this solution.

The theory is applied to pulsed waveforms. This section discusses 1) extending dose options from DC-based to pulses; and 2) formalizing computer-driven design (models) of wearable disposable electrotherapy. Building on the first-of-its-kind existing capability of Wearable Disposable Electrotherapy, these next steps leverage state-of-the-art bespoke electrochemical, manufacturing, and unprecedented computer simulation technologies.

Along with tDCS, iontophoresis, and wound-healing, there can be applications not traditionally DC-based that can be emulated by DC, such as DC-based paresthesia transcutaneous electrical nerve stimulation (TENS) devices. Nonetheless, the broad (universal) utility of Wearable Disposable Electrotherapy follows from the addition of pulsed waveform control. This section discusses two approaches: a) low-profile input/output (I/O) controller; and 2) Bare-die application-specific integrated circuit (ASIC). Each approach has relative advantages, requiring innovative but realistic technology; achieving either realizes pulsed waveform control wearable disposable electrotherapy. The models serve to verify and optimize wearable disposable electrotherapy designs.

This section discusses a theoretical framework governing device/load coupling (not strictly voltage or current controlled while supporting prescribed discharge range across subjects) and an associated numerical solution. The framework and solution can create anatomically-precise device-electrochemistry-coupled nonlinear/adaptive tissue models of discharge.

The 1) enhancement of wearable disposable electrotherapy to include pulsed waveforms; and 2) development of simulation tools to tune wearable disposable electrotherapy for any application would dramatically expand its application space and further broaden its impact. This section discusses a platform and customization tools needed to design and build wearable disposable electrotherapy as a replacement to any electronics-based non-invasive electrotherapy.

The applications of wearable disposable electrotherapy can span accelerated wound healing, peripheral or migraine pain management, transdermal drug delivery, or an extensive range of approved or investigated brain stimulation treatments. Irrespective of the application, as long as wearable disposable electrotherapy reproduces the functional dose (matching therapy effectiveness), it will categorically change usability, cost, and distribution factors.

Enabling steps for technology development are: 1) doses to pulsed stimulation; 2) models supporting application-specified system design, as wearable disposable electrotherapy can indeed be designed to emulate any existing non-invasive electrotherapy. The utility of pulsed waveforms is known.

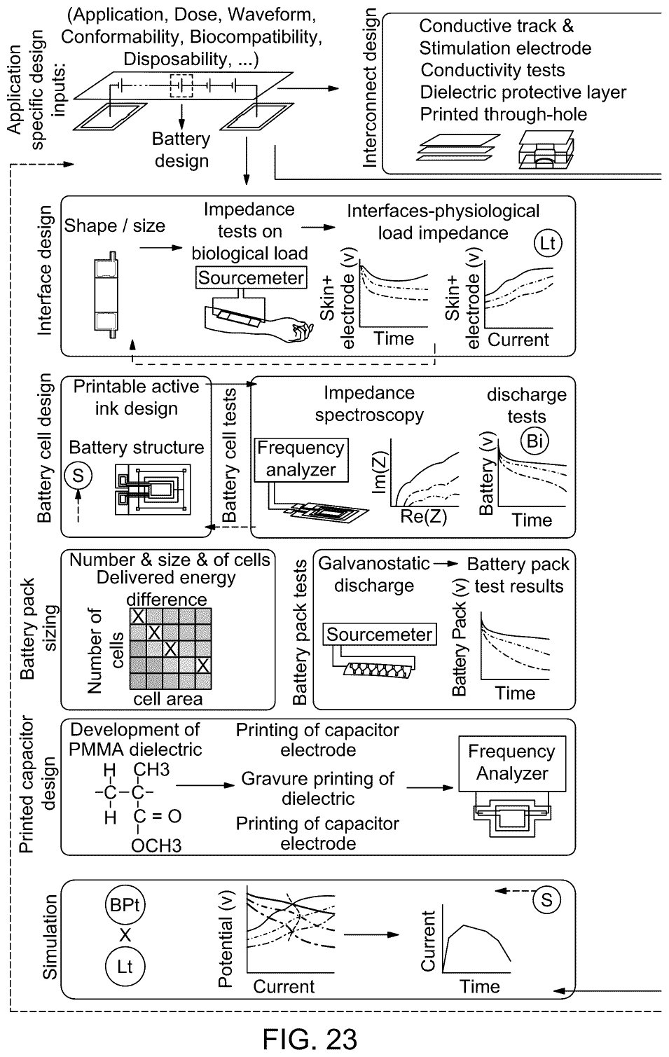

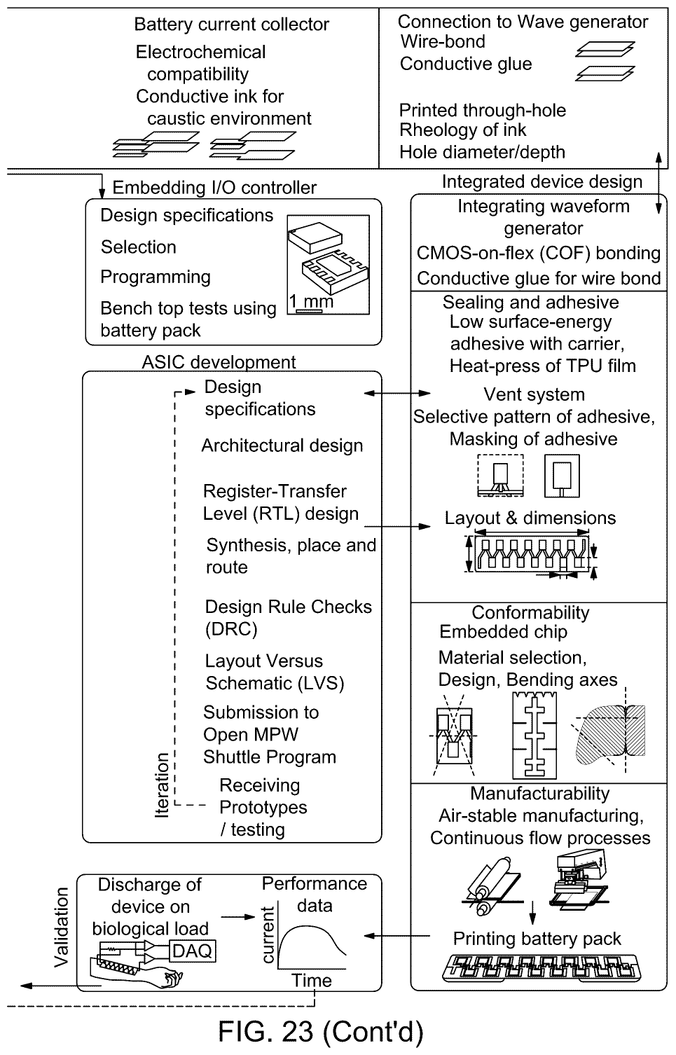

Replication of efficacy by wearable disposable electrotherapy requires includes emulation of applied dose, namely current over time and interface shape/position. Achieving the unique features of wearable disposable electrotherapy involved integrated design procedure to match the prescribed dose (FIG. 23). Wearable disposable electrotherapy design process is a theoretical framework to model and design performance.

For dose control, an implementation accounts for the nonlinearity of both energy sources (battery pack) and interfaces-physiological loads-unlike all prior electrotherapy devices with electronic output control. Described is a process that separately: 1) characterizes interface-biological impedance loads across subjects; and 2) develops self-regulated battery cells, which are sized into battery packs. Then, the discharge of a candidate battery pack design is simulated-supporting iterative battery/pack optimization, to ensure reliable output control across subjects. In order to simulate the interaction of the independently characterized (uncoupled) subsystems during operation (coupled), an isotemporal-trajectory theory is applied as: V_{B}(Iₙ, t|α_{B}, e_{B})=V_{L}(Iₙ, t|α_{L}, e_{L}) where the voltages V_{B} and V_{L} are the independently characterized battery pack and interfaces-physiological load subsystems, respectively, Iₙ is a constant current stimulus, α_{B} and α_{L} are internal parameters of each system, and e_{B} and e_{L} are environmental factors affecting each subsystem.

Simulations are used for wearable disposable electrotherapy design. Battery cell design is complex and iterative involving architecture, anode/cathode inks, current collectors, separator membrane, and electrolyte. And as single batteries have insufficient potential for delivering dose, a battery pack consisting of a series of batteries is required. Battery packs can contain the required amount of energy and a self-limiting mechanism, with an initial (peak) voltage matched to the compliance voltage limit of the interfaces-physiological load tests. To inform this complex design process with models, the approach developed is based on experimental data from the two uncoupled subsystems (battery pack and interfaces-physiological load) and then the behavior of the coupled system is simulated using isotemporal-trajectory theory. While the existing approach provided sufficient for the initial three DC-based applications considered, its current state is essentially “1D” with a lumped parameter battery pack connected at one point to the lumped parameter tissue. AC applications expand isotemporal-trajectory theory to 3D which requires non-linear (adaptive tissue and electrochemical) FEM models.

Advances in neuromodulation FEM from the first MRI-derived models of transcranial stimulation to high resolution cellular/skin models, extensions to adaptive tissue models, bioheat a range of multi-physics and advanced battery technologies. These combined with isotemporal-trajectory theory underpin the creation of wearable disposable electrotherapy models.

The wearable disposable electrotherapy device is configured to emulate the output of existing devices with a superior delivery platform. As such an enabling functional features to validate in human trials is the dose delivery (with the understanding that multi-stage clinical trials across any number of indications would then be justified over many years).

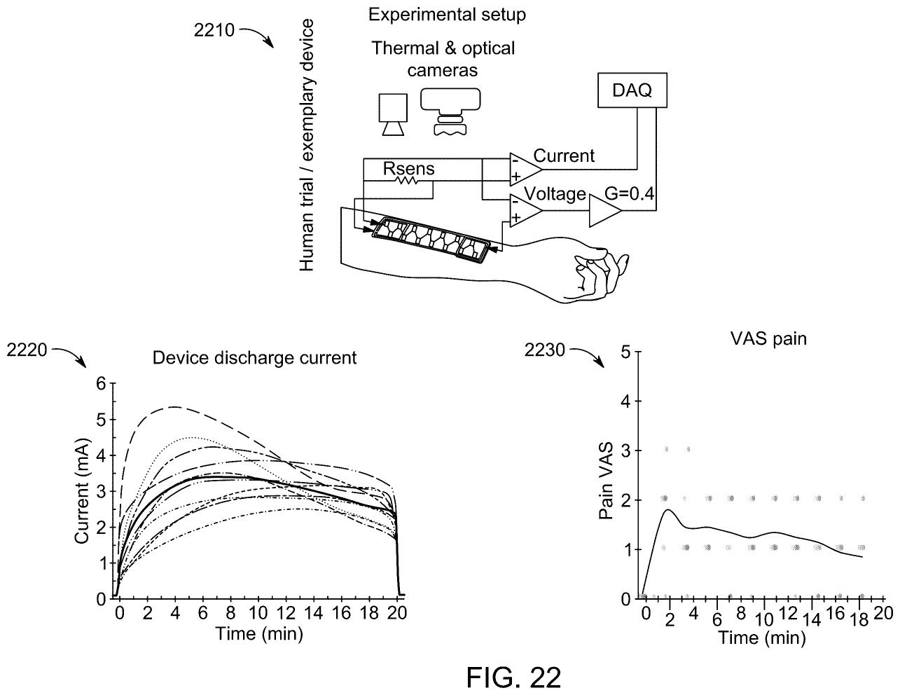

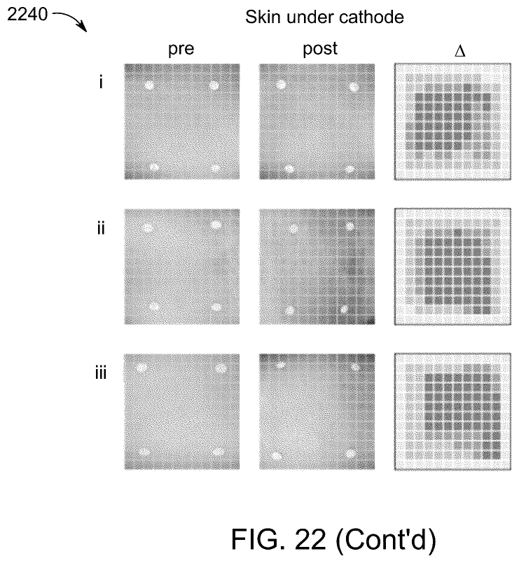

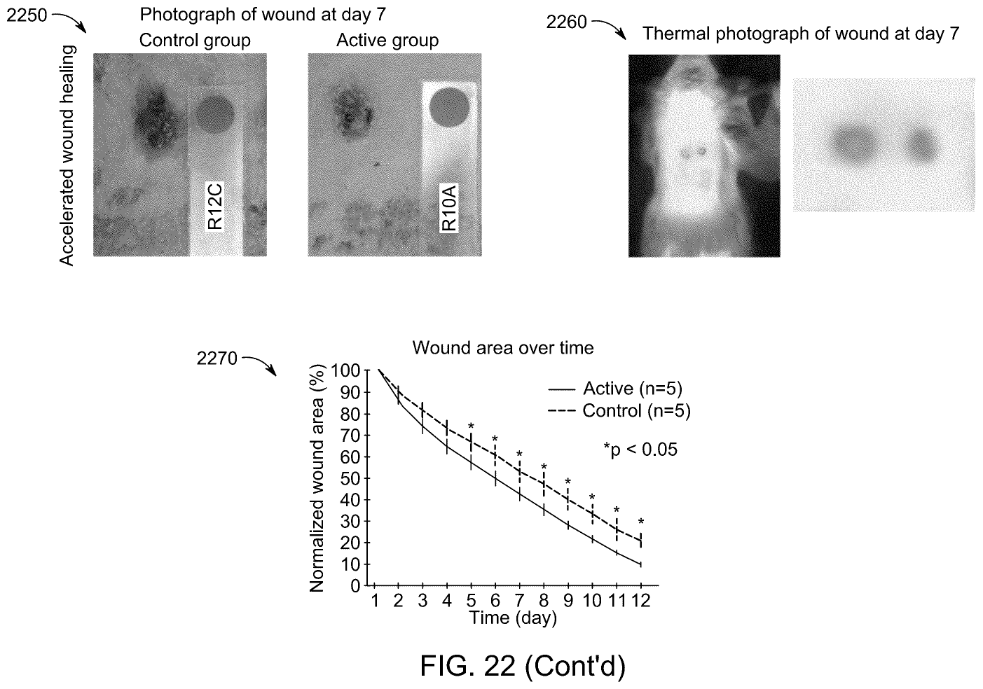

FIG. 22 shows images related to human and preclinical trials of the wearable disposable electrotherapy device. This section addresses creating and validating enabled technology for pulsed wearable disposable electrotherapy, including human trials of device output. Human trials were completed for DC-waveform wearable disposable electrotherapy (diagram 2210, plot 2220, plot 2230, plot 2240). Preclinical DC-waveform studies show efficacy for accelerated wound healing (photographs 2250 and 2260, plot 2270).

The described disposable electrotherapy device capable of pulsed stimulation that can compete in cost and usability with tablet pharmaceuticals, a categorical, technical and use-case innovation.

The device can eschew conventional electronics (e.g., circuit components, heavy metals) which hinder the development of environmentally responsible, single-use disposable devices. Instead, the therapeutic dose and requisite voltage can be controlled h a 3D electrochemical printed structure (10± layers in a millimeter-thin 3D battery pack structure) including modular battery cells with interconnects. Dose intensity and duration can be managed through exact control of active material quantities (micrograms of zinc), formulation of active materials, battery sizing (adjusting areal energy density relative to thickness), and dynamic power-load interface design considering the progressive impedance changes associated with device application and removal.

The common (embedded) substrate for all power/interface components can remove steps by the user (“no assembly required”). Devices can be activated upon contact, where the body completes the device discharge circuit (FIG. 1). Therefore, to use the device, can need only to apply it (e.g., absence of any controls, even a start button). In some implementations, the wearable disposable electrotherapy device can use an external start mechanism (e.g., pressing a button or peeling a sticker to expose conductive contacts).

Consequent to these features, wearable disposable electrotherapy devices can be distributed and used as economically and simply as pharmaceuticals or topical creams. This contrasts with other efforts creating increasingly complex wearable electronics. Aim 2 produces a turn-key modeling pipeline to design and optimize wearable disposable electrotherapy to any application.

The described computational workflow combines the core innovation of isotemporal trajectory theory with state-of-the-art tools in numerical solutions for multiscale and multiphysics simulations. A unique combination of features (adaptive meshing, adaptive tissue and input conditions, bioheat) produces models with unprecedented capability.

Embedding bare-die ASICs in labels are adapted from packaging technology, demonstrating the required reliability for use in medical devices while being suitable for disposable applications.

By leveraging the mature 130 nm CMOS fabrication process, along with wafer thinning techniques, flip-chip bonding, and flexible substrate integration, an embodiment is disposable, low-cost wearable devices compatible with roll-to-roll (R2R) production methods for wearable disposable electrotherapy devices. This designs aligns with the overarching goals of wearable disposable electrotherapy development by enabling high-throughput manufacturing, with environmentally-benign and abundant materials, and cost efficiency-supporting a single-use disposable. This approach can adapt technology has been adopted by the packaging industry for cost-sensitive disposable radio frequency identification (RFID) tags used in retail inventory management. In one implementation, a conventional RFID chip utilized in these labels measures approximately 502×720×75 μm and is integrated onto flexible printed adhesive labels, with a unit cost of ˜$0.20. In one implementation, the labels measure approximately 100×500×75 μm. In one implementation, the labels measure approximately 1000×500×75 μm. In one implementation, the labels measure approximately 1000×500×1000 μm. The dimensions are designed according to the overall device dimensions and applications.

An experiment emulated the hardware operation using commercially available I/O controllers with minimal hardware footprints, such as devices measuring 2.0×2.0×0.6 mm in a 8-UFDEN package. In one implementation, devices measure 2.0×5.0×0.6 mm. In one embodiment devices measure 10.0×5.0×6 mm. In one implementation, devices measure 20.0×5.0×50 mm, This approach enables the efficacy of printed wearable disposable electrotherapy devices with pulsed-output for identified applications.

Characterizing Impedance of Electrode and Skin: To effectively deliver the required dose, one first investigates the interaction between the battery pack with waveform generator and the load-which includes electrodes and skin impedance where electrodes are placed on the target area. Isotemporal trajectory theory is applied to specific pulsatile waveforms, for three exemplary applications. This includes at least two steps. First, characterization of the impedance of the electrode/skin in human trials using a commercial source-meter. Current-controlled pulses with application specific frequency and pulse structure are applied across the interface (hydrogel and stimulation electrodes) to targeted area, with varied pulse amplitude (Iₙ), and the associated voltages during the pulse train recorded. The process can be repeated across subjects generating the load impedance data needed for isotemporal modeling and associated device design. The second step is device characterization follows prototyping.

Defining Design Specifications/dose, Programming and Bench Testing: Establish the technical requirements for the I/O controller, including electrical specifications (voltage levels, current handling, number of I/O pins, package dimensions. Selection of a commercially available I/O controller that meets these criteria. Develop firmware to generate the defined dose of electrical stimulus.

The design includes device miniaturization. One step is to employing die thinning techniques such as mechanical grinding, chemical mechanical polishing (CMP), or laser etching to reduce the thickness of the waveform generator, enhancing flexibility.

Integration with Printed WDE Device: Using printed conductive glue for connection of wire bonding of thinned chip to printed conductive traces on the WDE substrate. Encapsulate the assembly during the sealing process and sandwich it between layers of the WDE's flexible substrates. The integration can be combined with verification that electrical performance is maintained under flexing and bending conditions typical of user movements.)

Post-Fabrication Benchtop Performance Testing: Conduct benchtop tests to verify performance of battery (packs) with embedded waveform generators under various load conditions. This characterization is the second data step of isopotential trajectory theory and supports simulations.

The design process includes human trials and performance recording: Conduct trials to assess the output performance of the WDE device with the embedded waveform generator. Collect and analyze data on battery/wave generator performance and delivery of required dose.

Die thinning techniques can introduce mechanical stress, potentially leading to defects or reduced reliability which could result in device failures. Achieving reliable electrical connections between the thinned chip and printed conductive traces is technically challenging. Poor connections can lead to increased resistance or device failure. as Alternative interconnects can include anisotropic conductive films (ACF) or conductive adhesives. Rigid and flexible conductive adhesives show different failure points, the optimum solution can be found by further testing. The layout also can be adjusted in the device design stage to minimize stress on interconnects during flexing. A single (universal) commercially available I/O controller that meets all technical requirements (size, operational voltage range, current handling, power consumption, internal clock generation) may be used. In an alternative embodiment applications-specific controllers are used or by adaptation of stimulation waveform (eg. increased pulse width) requiring lower voltage ranges.

Voltage adaptation for wearable disposable electrotherapy using pulses stimulation can be enabled by integrating: 1) the associated interface and physiological load range; 2) the nominal pulse with and intensity; and 3) the modified pulse with and intensity. The nominal pulse with and intensity are defined by the functionality of a conventional device such as one that depends on electronics, or packaged batteries, or current control electronics or some combination thereof. Nominal pulse with and intensity is 10 microseconds to 800 microseconds, and 0.1 mA to 100 mA. Nominal pulse with and intensity is 100 microseconds to 600 microseconds, and 0.5 mA to 60 mA. Nominal pulse with and intensity is 150 microseconds to 300 microseconds, and 1 mA to 8 mA. The associated interface and physiological load range is determined according to the methods described. Case A: The modified pulse width and intensity for a nominal pulse with and intensity is 10 microseconds to 800 microseconds, and 0.1 mA to 100 mA and a resistance of 500 to 10000 ohm is 100 microseconds to 8000 microseconds, and 0.1 mA to 10 mA. Case B: The modified pulse width and intensity for a nominal pulse width and intensity is 100 microseconds to 600 microseconds, and 0.5 mA to 60 mA and a resistance of 500 to 10000 ohm is 300 microseconds to 10000 microseconds, and 0.1 mA to 10 mA. Case C: The modified pulse width and intensity for a nominal pulse with and intensity is 150 microseconds to 300 microseconds, and 1 mA to 8 mA and a resistance of 500 to 10000 ohm is 300 microseconds to 600 microseconds, and 0.1 mA to 5 mA. A current specified can be associated with a voltage so that requirements can be applied to voltage. The modified pulse with and intensity is associated with a designed battery pack output voltage (e.g. number of cells and design). For case A the battery pack voltage is less than 80 V, preferably less than 60 V, still more preferably less than 20 V. For case B the battery pack voltage is less than 60 V, preferably less than 30 V, still more preferably less than 14 V. For case C the battery pack voltage is less than 30 V, preferably less than 15 V, still more preferably less than 10 V. The duty cycle of the modified pulse is increased compared to the nominal pulse by a factor of 2×, 10×, 20×, or 50×. The duty cycle of the modified pulse can range from 1% to 100%. Increasing the duty cycle can increase the pulse width for a given frequency. At 100% duty cycle the waveform can be said to be continuous. In one implementation, the waveform is continuous, 10-150 Hz, with a battery pack voltage of less than 60 V. In one implementation, the waveform is continuous, 100-150 Hz, with a battery pack voltage of less than 40 V. In one implementation, the waveform is continuous, 1-80 Hz, with a battery pack voltage of less than 30 V.

The design process can include ASIC design of the waveform generator and bench testing. For defining design specifications, one step it so specify the ASIC's functional requirements, including output voltage range, required pulse programs, power consumption targets, die size constraints, and compliance with medical device regulations.

The device design process can include architectural and RTL design including the step to develop the ASIC's architectural design, outlining the power output and digital blocks required (e,g., oscillators, amplifiers, digital control logic). Proceed with Register-Transfer Level (RTL) design using hardware description languages like Verilog or Very High-Speed Integrated Circuit Hardware Description Language (VHDL).

The device design process can include synthesis and Place-and-Route: Use Electronic Design Automation (EDA) tools such as Yosys for logic synthesis and OpenROAD for place-and-route to transform the RTL design into a gate-level netlist and then into a physical layout, adhering to SkyWater's 130 nm process design rules.

FIG. 23 illustrates a plan and design pipeline for a wearable disposable electrotherapy device. A comprehensive workflow and theory support design and validation of application-specific wearable electrotherapy devices. Each design stage (colored regions) incorporates constraints derived from wearable disposable electrotherapy features, design inputs (application-specific dose), and design outputs from other stages and produces design outputs. As the outcomes of each stage impact other stages of the design, device design is integrated and iterative. Design outputs/elements (bold). Integrated workflow (solid arrows). Verification/validation (dashed arrows). Elements adapted from DC-waveform devices marked in gray while elements/analysis for pulsed waveform marked in colors.

The design can include rule checks and manufacturing submission: Perform comprehensive Design Rule Checks (DRC) and Layout Versus Schematic (LVS) verification to ensure manufacturability and correctness of the design. Prepare and submit the final GDSII files to the Open MPW Shuttle Program for fabrication.

Post-Fabrication Benchtop Performance Testing: Upon receiving the fabricated ASICs, conduct electrical testing using printed batteries and integrate the ASIC into the wearable disposable electrotherapy device's circuitry. Validate the waveform generation, output voltages, and overall device functionality under various load conditions. This characterization is also the second experimental step for isopotential trajectory theory used in simulations.

As part of iterative refinement, the design step includes analyze test results to identify any deviations from specifications and areas for refinement. Update design accordingly and prepare for subsequent fabrication runs as necessary.

A further step in device design is human trials and performance recording: Conduct trials to assess the output performance of the wearable disposable electrotherapy device with the embedded waveform generator. Collect and analyze data on battery/wave generator performance and delivery of required dose.

Achieving higher operational voltages (at least 10 V) in a 130 nm complementary metal-oxide semiconductor (CMOS) process can be device limiting; extended-drain n-channel metal-oxide semiconductor (NMOS) and p-channel metal-oxide semiconductor (PMOS) transistors designed for higher voltages can mitigate the voltage compliance problem. ASIC fabrication can result in low yields, with a portion of chips being non-functional due to defects. The design can be resilient to low yields by implementing specific design practices. A further solution for low yield is to include redundant circuits where feasible to mitigate the impact of defects. Due to die size limitations of the MPW shuttle program, sub-mm size bare-dies are impractical in this stage which is required for integration with printed battery packs. In the large design space of ASIC chips for implementation of active components, implementation of multiple designs for different applications is possible allowing parallel design of devices for different applications. The size of ICs can prompt fabricating connections between the printed battery pack and ASIC chip and stimulation electrodes for benchtop tests without embedding in the wearable form factor of WDE.

As part of inventing wearable disposable electrotherapy, a theoretical framework governing device/load coupling (unique at output is not strictly voltage or current controlled) was developed. Isotemporal-trajectory theory involved independent experimental characterization of the non-linear interface/physiological load and of the device battery packs, and then simulating their coupled discharge. This theory was successful in designing three DC-based applications and was validated in the associated prototype tests. This section extends isotemporal-trajectory theory to pulsed-stimulation (and from a 1D to a 3D numerical solution. This analysis can be combined with the determination of modified pulse with and intensity.

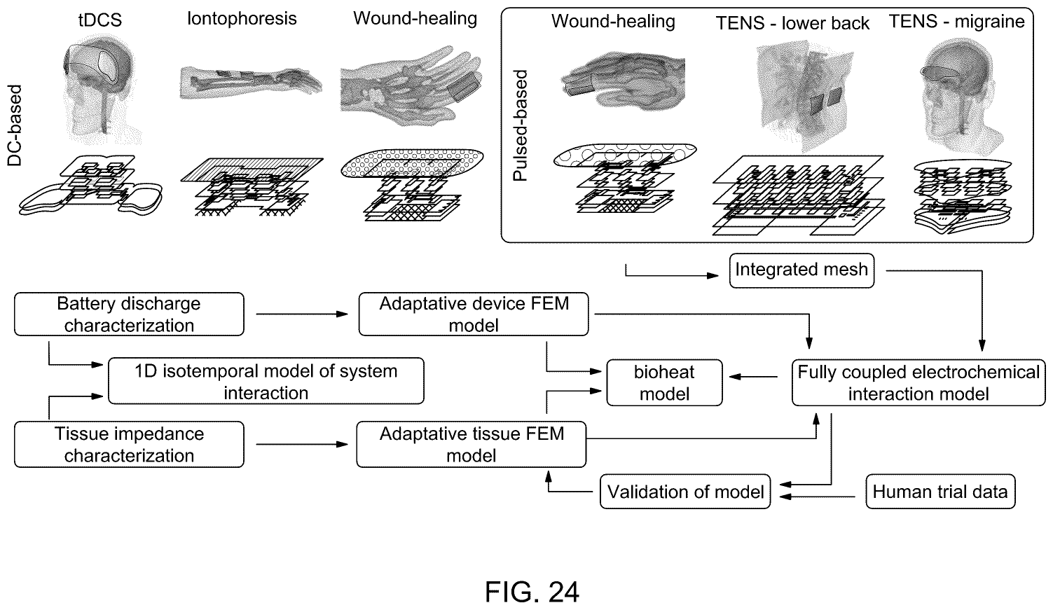

FIG. 24 illustrates an end-to-end pipeline for simulations and design of wearable disposable electrotherapy to emulate any conventional device output. The computational workflow combines the core innovation of isotemporal trajectory theory (developed and validated first for DC applications) with state-of-the-art sophistication in numerical solutions allowing multiscale and multiphysics simulations. Data fcan be integrated as battery and tissue impedance data, and final model results are verified against experimental wearable disposable electrotherapy trials. Developed for any application, verification focuses on three exemplary pulse-based applications. Experimental data can constrain both model inputs and outputs, while a unique combination of features (adaptive meshing, adaptive tissue and input conditions, bioheat) produce models with unprecedented capability.

Modeling tool development in combination with the unique wearable disposable electrotherapy discharge physics enables anatomically-precise nonlinear/adaptive tissue models of discharge. These modeling tools can verify existing approaches and optimize ongoing design. The models was unprecedented in combining these features.

Extend 1D isotemporal-trajectory theory to pulsed stimulation. The experimental and numerical techniques developed and validated for DC-based applications, are extended to any pulsed stimulation and demonstrated explicitly for three exemplary applications (accelerated wound healing, back-pain TENS, migraine TENS). For each application dose, electrode/body impedance data from multiple subjects and battery-cell discharge data are combined to predict current output across subjects. Developed and validated for DC, isotemporal-trajectory theory is already explicit in time and device/load non-linearities for AC. The governing equation for isotemporal-trajectory theory, V_{B}(Iₙ, t|α_{B}, e_{B})=V_{L} (Iₙ, t|α_{L}, e_{L}) can follow from the voltage/current of the device and of the much match throughout discharge. This can support predictions to be agnostic to internal states (α_{B}, e_{B}, α_{L}, e_{L}) so long as the device discharge and device/interface impedance are characterized with sufficient resolution (I-Iₙ is small).

The design steps can include to develop explicit device structure models (CAD-derived) and associated tissue models for exemplary applications. For the device models resulting structural elements include conductive tracks/interconnects, stimulation electrodes and hydrogel, battery structure (electrolyte, electrodes), sealants/adhesive, enclosure, and structural/venting modifications, producing models with ˜33 M elements. For tissue models, representation of gross anatomy and multi-skin layers (relevant for interface/adaptive tissue models) will produce models of ˜50 M elements. A fast imaging employing steady-state acquisition (FIESTA) and spin echo sequences was deployed to obtain high-resolution skin scans. The skin multi-layer model comprise mosaic pattern, folding, and ridge patterns, and dimensions (thickness and diameter) of multi-layers including keratinous stratum corneum, epidermis, dermis, and hypodermis (fat and muscles), and ultra-structures such as sweat glands, hair follicles, and macro-capillary bed. Structures that are small then image resolution are integrated from realistic CAD based on established anatomical models. Each anatomical model is derived from high-resolution MRI optimized for the region, leveraging existing data and pipelines.

Mesh generation and model convergence. The volumetric tissue and device mesh was generated from a voxel-based tetrahedral adaptive meshing solver algorithm to enhance computational efficiency and accuracy. This iterative adaptive mesh refinement algorithm can automatically adjust the mesh density based on error estimates from an initial analysis on a coarse tissue and device mesh, can automatically estimate discretization error, can automatically refine areas with high error and coarsens areas with low error to generate a new mesh, and repeats the analysis on the new mesh until an optimal convergence criterion is met. For a time-dependent solution, first an adapted solution at t=tₙ was mapped to the coarse base mesh. Next, a new adapted mesh can be obtained by using the error indicator sampled at given points in [tₙ, tₙ₊₁], selecting a set of elements based on the element pick function, and then finally refining these elements. The solution at tₙ to the partial differential equation problem on the previously adapted mesh for [tₙ₋₁, tₙ] is then mapped to the new mesh for [tₙ, tₙ₊₁] and time integration continues until the next mesh adaptation takes place at tₙ₊₁. The amount of mesh refinement was determined using a measure ρ=1/2PNΣ_{i=1,γ (i)≠0}^{N} 2^{γ(i)} where γ is an N-vector of integers containing the number of times the element at the position should be refine, ρ=maxᵢγ (i), and N is the number of elements in a base coarse mesh⁸. Model convergence, typically attained faster with adaptive methods compared to uniform mesh refinement, was assessed by tracking relative change in voltage at tissue and device compartment between refinement iterations, setting an allowable ˜5% change in tolerance, and monitoring both local and global error estimates. The resulting adaptive mesh of complex multi-compartment device and tissue models will capture ˜30 μM resolution for devices, ˜100 μM for skin, and ˜500 μM for deeper tissue.

Adaptive tissue models can be used to optimize device design. Complex non-linear properties of skin were recognized for decades are coupled into FEM models-thus capturing the spatial and temporal adaptation of tissue to electrical stimulation. Such consideration is important in wearable electrotherapy discharge, where discharge is regulated by the coupling of device and tissue properties. Building on representation in 1D models, adaptive tissue properties can be applied FEM. The approach is based on application-specific interface/physiological impedance measurements parameterizing adaptative skin responses. While these responses are complex, the approach successfully developed for ECT can be adapted whereby for each tissue class (e.g., epidermis, fat) a transfer function between local electric field (Eₛₛ) and the adapting conductivity (Oss) is developed, assuming a monotonic dependence with just 4 parameters: the minimum tissue conductivity (e.g., in the absence of electric field), the threshold electric field for conductivity changes, the maximum tissue conductivity, and the electric field the maximum conductivity is achieved. These parameters are in turn derived prior literature and fitting to the expansive set of experimental observations. Though resulting stimulation responses are nonlinear, pseudo-quasistatic calculations can be used.

Whereas in 1D isotemporal models, the device and tissue are coupled by a single line, for 3D models the adaptive properties of the batteries are modeled though the source boundary conditions. This innovative approach avoids explicit electrochemical modeling of batteries while leveraging 1D isotemporal-trajectory theory to incorporate measurement-constrained at an appropriate level of representation. First, the 1D iso-temporal-trajectory theory was implemented to model adaptive properties of the device, thereby computing the potential and current distribution in the device by adjusting its charging and discharging behaviors to optimize performance and longevity as well as coupling heat transfer. To effectively model battery output dynamics, a lumped battery interface module can be used with initial conditions set based on initial state of charge (SOC) and voltage based on the fully charged state of the device. Next, a time-dependent function was defined to adjust the battery voltage based on discharge data. A charge-discharge cycling feature can set up load cycles that include rest periods and specific current thresholds. Voltage losses can be incorporated by specifying parameters such as ohmic, activation, and concentration overpotential to accurately reflect real-world performance of the device. Finally, a time-dependent solution can be computed to predict current output over time with voltage adjustment. This dynamically adjusted output was applied as a Neumann boundary condition (active) and Dirichlet boundary condition (return) to the FEM model.

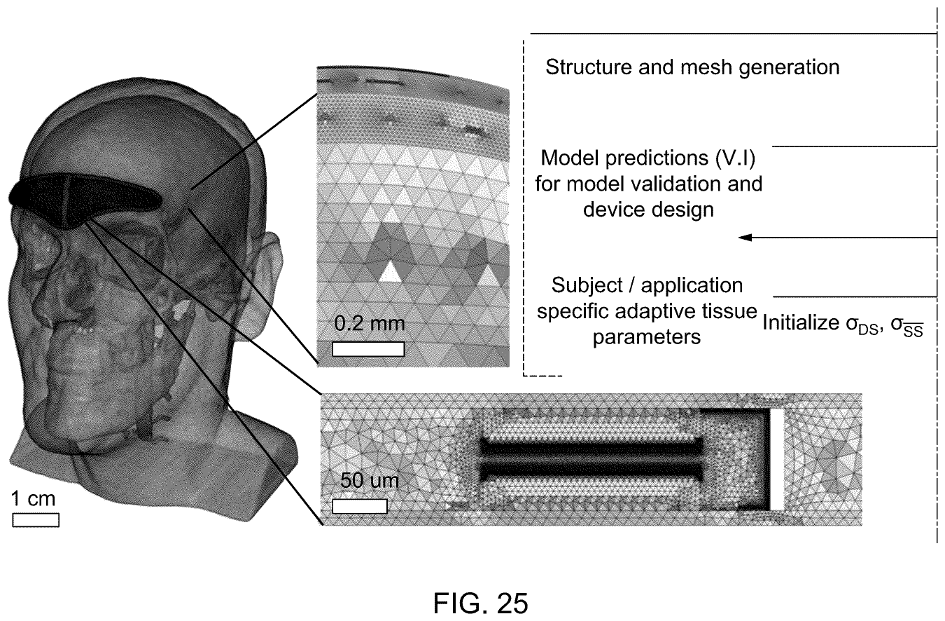

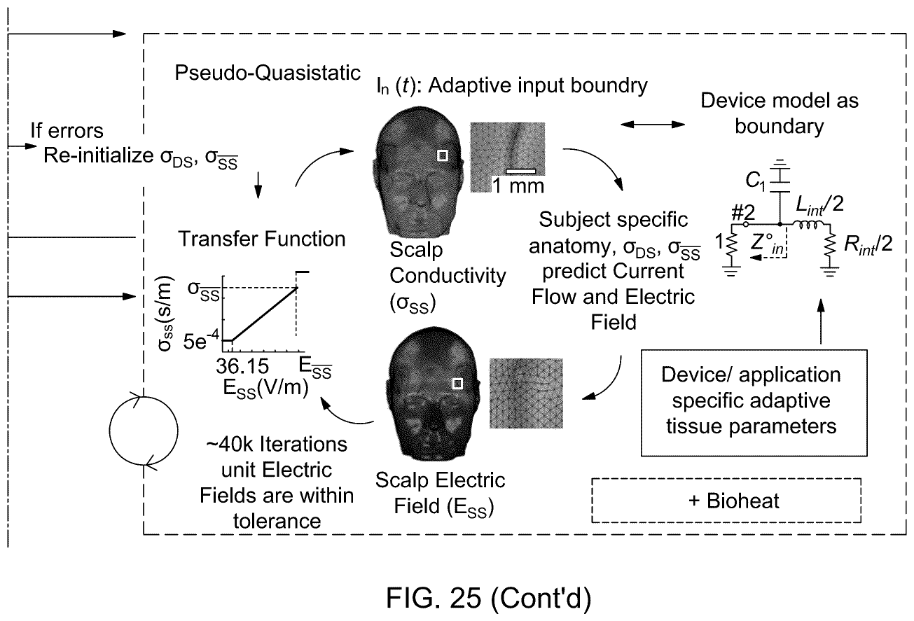

FIG. 25 illustrates adaptive meshing and computation. The research strategy can develop modeling elements incrementally building toward an unprecedented multi-scale multi-physics platform of electrotherapy stimulation, and specifically for wearable disposable electrotherapy design.

Device and tissue bioheat. A bioheat simulation is used for wearable disposable electrotherapy. While injurious, or even significant, heating is not expected, even moderate (e.g. 1° C.) changes can impact tolerability, impedance, and device electrochemical performance. Adapting techniques previously developed for the unique wearable disposable electrotherapy architecture and discharge characteristics, device heating is modeled considering local and superficial heat generation and conduction, while for tissue, blood flow and temperature-dependent metabolism are incorporated. Electric potential generated from the stimulation will then be coupled with Penne's Bioheat equation to estimate tissue heating as:

$$ρ {C}_{p} \frac{\partial T}{\partial t} = ∇ . ( κ ∇ T ) - {ρ}_{b} {C}_{b} {ω}_{b} ( T - {T}_{b} ) + {Q}_{met} + {Q}_{ext} + σ {\vert ∇ V \vert}^{2}$$

where ρ, Cₚ, T, T_{b}, κ, ρ_{b}, C_{b}, ω_{b}, Qₘₑₜ, and Qₑₓₜ represent tissue density (kg-m3), specific heat (J·kg-1·K-1), temperature (K), blood temperature (K), thermal conductivity (W·m-1·K-1), blood density (kg·m-3), blood specific heat capacity (J·kg-1·K-1), blood perfusion rate (s-1), metabolic heat generation rate (W·m-3) 44, internal device electrochemical heat generation (W·m-3), and Joule heating due to stimulation (σ|∇V|²), respectively. Evidently inside the device, perfusion terms are absent. The superficial heating from the device surface was modeled as a Neumann thermal boundary condition to the fully coupled bioheat model.

A dynamic tissue conductivity as a function of electric field strength, number of stimulus, and joule heat can be adapted to capture the non-linear tissue-specific behavior and responses to electrical stimulation. Notwithstanding that modeling elements are nonlinear, the numerical competition can follow pseudo-quasistatic (FIG. 25). The Laplace equation can be used to solve for electric potential as: ∇·(σ(E, N, T)∇V)=0 and E=−∇V where V is voltage and σ is the tissue conductivity as a function of electric field (E), number of stimulus (N), and temperature (T).

Data from human trial prototype validation validate model outputs at all stages of development. Mismatches are opportunities to refine model parameters and methods.

The design process addresses convergence issues due to model instability, computationally expensive for complex 3D models, and incompatible with all element types or analysis features in some solvers. Adaptive mesh refinement refines and optimizes element sizes in critical model areas. A Boundary Element Method (BEM), an alternative to FEM modeling, was implemented for computational efficacy, prediction accuracy, mesh generation simplicity, unique solution characteristics, and handling of singularities. The pipeline does not include physiological response modeling (e.g., neural stimulation) as this has no impact on the use of the models, which is to predict wearable disposable electrotherapy output. But given models predict electric field the pipeline can be extended to any physiological/systems/disease computational models.

There are evidently gaps in the validation of each non-invasive electrotherapy with some technologies such as TENS for pain proven over decades, and others such as tDCS for age-related cognitive decline, still investigational. However, every single device has the same limiting form factor: a durable electronics device that needs batteries and programming, cables, and attachment to disposable electrodes. wearable disposable electrotherapy is the first and only electrotherapy can be distributed like drugs or topical creams, as dose/indication specific adhesive strips. A patient can carry a single device, simply apply it discreetly when needed, and then discard it. Therefore, irrespective of the stage-in-development of a given electrotherapy, its distribution can be categorically enhanced by adoption into a wearable disposable electrotherapy platform—making this proposal impactful across hundreds of technologies and indications.

Electrotherapy dose is “built-into” device 3D architecture/chemistry, with discharge initiated simply by applying the device. We develop an associated theoretical framework for device design based on coupling between load and battery pack, with all elements and manufacturing processes environmentally-benign and scalable at low-cost.

In the descriptions above and in the claims, phrases such as “at least one of” or “one or more of” can occur followed by a conjunctive list of elements or features. The term “and/or” can also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results. For example, the logic flows can include different and/or additional operations than shown without departing from the scope of the present disclosure. One or more operations of the logic flows can be repeated and/or omitted without departing from the scope of the present disclosure. Other implementations can be within the scope of the following claims.

What is claimed is:

1. A wearable device, comprising:
a substrate layer, the substrate layer configured to support:
a battery array comprising a plurality of battery cells, wherein at least a first battery cell is electrically coupled to a second battery cell using a conductive interconnect; and
an interface layer coupled to the substrate layer, wherein the interface layer is electrically coupled the battery array via a translayer interconnect, the interface layer configured to adhere the substrate layer to a body and to provide electrical stimulation to the body.

2. The wearable device of claim 1, wherein the first battery cell of the battery array is staggered from the second battery cell of the battery array such that an axis of bending is present between the first battery cell and the second battery cell.

3. The wearable device of claim 1, wherein the substrate layer is generated by printing.

4. The wearable device of claim 1, wherein the first battery cell and the second battery cell are connected in series.

5. The wearable device of claim 1, wherein the translayer interconnect couples the interface layer and the battery array via a through-hole, wherein the through-hole comprises a perforation of the substrate layer.

6. The wearable device of claim 5, wherein the through-hole comprises a metallic ink.

7. The wearable device of claim 1, wherein a battery cell of the plurality of battery cells comprises an anode and a cathode.

8. The wearable device of claim 7, wherein the anode and the cathode are stacked in a pair.

9. The wearable device of claim 7, wherein the anode comprises a metal.

10. The wearable device of claim 9, wherein the metal is zinc.

11. The wearable device of claim 7, wherein the cathode comprises a metal oxide.

12. The wearable device of claim 11, wherein the metal oxide is manganese dioxide.

13. The wearable device of claim 7, further comprising a separator membrane between the anode and the cathode.

14. The wearable device of claim 1, wherein the substrate layer comprises a polymer.

15. The wearable device of claim 14, wherein the polymer is polyethylene terephthalate (PET).

16. The wearable device of claim 1, wherein the interface layer comprises an electrode and an ion-conductive buffer.

17. The wearable device of claim 16, wherein the ion-conductive buffer comprises a hydrogel sheet or a non-woven sponge.

18. The wearable device of claim 16, wherein the electrode comprises an anode and a cathode.

19. The wearable device of claim 1, wherein the conductive interconnect comprises a copper track beneath a carbon conductive track, disposed underneath a dielectric layer.

20. The wearable device of claim 1, further comprising a gas channel in fluid communication with a battery cell of the battery array, the gas channel providing a vent to a space outside of the substrate layer.
